(19) United States
(12) Patent Application Publication
(10) Pub. No.: US 2018/0326022 A1
(43) Pub. Date: Nov. 15, 2018

(54) COMPOSITIONS AND METHODS FOR IMPROVING HEART FUNCTION AND TREATING HEART FAILURE

(71) Applicant: The Trustees of the University of Pennsylvania, Philadelphia, PA (US)

(72) Inventors: Benjamin L. Prosser, Philadelphia, PA (US); Kenneth B. Margulies, Villanova, PA (US)

(21) Appl. No.: 15/959,181

(22) Filed: Apr. 21, 2018

Related U.S. Application Data
(60) Provisional application No. 62/488,429, filed on Apr. 21, 2017.

(51) Int. Cl. A61K 38/48; A61P 9/04; A61K 48/00; A61K 31/365

(52) U.S. Cl. CPC A61K 38/48; A61P 9/04; A61K 48/0066; A61K 31/4184; C12Y 603/02025; C12Y 304/17017; A61K 2300/00; A61K 31/365

(57) ABSTRACT

Described herein is a method for improving or stabilizing cardiac function by inhibiting tubulin carboxypeptidase (TCP). Also described herein is a method for treating heart failure in humans comprising dosing a patient with a therapeutic which interferes with detyrosinated microtubules in cardiomyocytes. Also provided are viral vectors which comprise a nucleic acid encoding a tubulin tyrosine ligase (TTL) gene under the control of regulatory elements direct expression thereof. Compositions are also provided which contain such viral vectors formulated for delivery to a human patient.

### CROSS-REFERENCE TO RELATED APPLICATIONS

This application claims the benefit of the priority of U.S. Provisional Application No. 62/650,227, filed Mar. 29, 2018, which is incorporated herein by reference.

### STATEMENT OF FEDERALLY SPONSORED RESEARCH

This invention was made with government support under NHLBI R01 HL133080, HL089847, and HL105993 awarded by The National Institutes of Health (NIH). The government has certain rights in the invention.

### BACKGROUND OF THE INVENTION

Heart failure is defined as a failure of the heart to provide adequate blood flow to the organs and tissues of the body. Currently, there is no cure for heart failure, and existing therapies have shortcomings. Other approaches used in clinical settings to boost cardiac contractility (cardiac “inotropes”) have a marked limitation in that they are energetically unfavorable. For example, inotropes often target improved calcium cycling or increased force generation, which both require increased energy usage to fuel these ATP-dependent processes. In heart failure with reduced ejection fraction, the existing therapies for augmenting the heart's contractility have been associated with no survival benefit, with an increased risk of ischemia or arrhythmias, and are thus viewed as palliative. In contrast, targeting microtubules to lower internal resistance should be energetically favorable. This does not cause the muscle cell to intrinsically produce more force or cycle calcium faster, but simply lowers the internal resistance that normally opposes that force. This should allow the heart to do more work for the same amount of energy usage, a distinct advantage over other inotropic approaches.

Heart failure with preserved ejection fraction (or HFpEF), currently has no approved therapies, even though it is estimated to represent almost half of all heart failure cases. Many cases of HFpEF exhibit slowed relaxation of the heart muscle that contributes to abnormalities of pump function. Slowed myocardial relaxation in HFpEF may also be due to increased internal resistance attributable to microtubules (MTs), and targeting MTs might enhance relaxation rates without increasing energy usage.

Along with its well-defined transport functions, the MT network serves multiple mechanical roles in the beating cardiomyocyte. MTs function as mechanotransducers, converting changing contractile forces into intracellular signals (1, 2). MTs may also act as compression resistant elements, which could provide a mechanical impediment to cardiomyocyte contraction (3, 4, 5). If so, they must bear some of the compressive and tensile load of a working myocyte. Unfortunately, little is known about MT behavior during the contractile cycle. During this cycle, Ca²⁺ mediated actin-myosin interaction first shortens repeating contractile units called sarcomeres, which are then stretched as the heart fills with blood during diastole.

Although an isolated MT would present minimal resistance to myocyte compression, the stiffness of the network within a living cell, with microtubule associated proteins and other cytoskeletal elements, can change by orders of magnitude (6, 7). It is in this context that MTs are proposed to act as compression resistant elements that may impair sarcomere shortening and thus cardiac function, particularly in disease states associated with MT proliferation (6, 8, 9, 10). Post-translational modification (PTM) of MTs (11, 12) could also modify their mechanical properties and binding interactions. Detyrosination, a PTM of α-tubulin, has recently been shown to augment MT-dependent mechanotransduction in dystrophic cardiac and skeletal muscle (12). This specific PTM is also increased in animal models of heart disease (1, 13, 14), which raises a mechanistic question: if the MT network is altered, have the mechanical properties of the myocyte changed?

While the idea that a proliferated (and perhaps modified) MT network may mechanically interfere with contraction is attractive, the “microtubule hypothesis” has remained controversial (for reviews, see 15, 16).

There remains a need for treatment of heart failure, and particularly treatments which minimize the increased risk of ischemia or arrhythmias associated with current palliative efforts which require significant energy.

### SUMMARY OF THE INVENTION

Provided herein are compositions and methods useful for treating patients with heart failure to lower cardiac stiffness and for improving improve cardiac output. Our human studies suggest that improved contractility and relaxation from suppressing detyrosinated microtubules show more benefit in sicker patients, with more severe myocardial dysfunction, compared to early stage patients. However, there are benefits in early stage patients as well.

In one aspect, a method for treating heart failure in humans is provided. The method comprises treating a patient with a composition comprising a therapeutic which interferes with detyrosination of microtubules in cardiomyocytes. In certain embodiments, the therapeutic is a small molecule drug.

In one aspect, a method for improving heart function in humans is provided which comprises treating a patient with a therapeutic which inhibits tubulin carboxypeptidase (TCP). In certain embodiments, the therapeutic (active ingredient) may be sesquiterpene lactones, such as parthenolide or costunolide, or a prodrug, derivative, or analog thereof, or Epoy.

In another aspect, a method for treating heart failure in humans is provided which comprises dosing a patient with a therapeutic which interferes with detyrosinated microtubules in cardiomyocytes. The therapeutic may be a small molecule drug selected from one or more of: sesquiterpene lactones including parthenolide (PTL), costunolide or PTL pro-drugs such as LC-1, or microtubule destabilizers including colchicine, vinblastine, and nocodazole.

In another embodiment, the therapeutic comprises a nucleic acid encoding a tubulin tyrosine ligase (TTL) gene under the control of regulatory elements direct expression thereof. The nucleic acid may be delivered by a non-viral gene delivery system or by a viral vector.

In certain embodiments, a method for improving heart function in humans is provided. The method comprises delivering a composition comprising a therapeutic which increases cardiac microtubule tyrosination. The therapeutic comprises a nucleic acid encoding a tubulin tyrosine ligase (TTL) gene under the control of regulatory elements direct expression thereof. The nucleic acid may be delivered by a non-viral gene delivery system or by a viral vector. The viral vector may be a recombinant adenovirus, lentivirus, or adeno-associated virus.

In certain embodiments, a replication-defective vector is provided which comprises a tubulin tyrosine ligase (TTL) under the control of a regulatory control sequence which directs expression thereof in the heart.

Also provided are compositions containing such a vector suspended in a suitable vehicle.

Other aspects and advantages of the present invention will be apparent from the following Detailed Description of the Invention.

### BRIEF DESCRIPTION OF THE FIGURES

FIG. 1A-FIG. 1J. Microtubules reversibly buckle in contracting cardiomyocytes. (FIG. 1A) The subsurface (top) and interior (bottom) cardiomyocyte microtubule network. (FIG. 1B) High speed confocal imaging of MTs at rest (top) and during contraction (bottom) labeled with SiR Tubulin with brightness increased for comparison with (FIG. 1C). (FIG. 1C) Airyscan imaging of the same MTs as in (FIG. 1B) at rest and during contraction. (FIG. 1D) Wider view of MTs labeled with EMTB-3xGFP at rest (FIG. 1D, top) and during contraction (FIG. 1D, bottom). (FIG. 1E) Microtubules imaged throughout a contractile cycle (cyan) were overlaid onto the network configuration from the initial frame at rest (red). (FIG. 1F) Colocalization analysis of (FIG. 1E) shows that MTs repeatedly return to the same position. Pearson's coefficient is used to estimate goodness of fit to original microtubule configuration over several contractile cycles. Initial drop to ˜0.96 is due to imaging noise. (FIG. 1G) Quantification of buckling amplitude (measured from centerline to edge) and λ (measured as twice the distance between consecutive inflection points). (FIG. 1H) Amplitude of MTs labeled with EMTB-3xGFP in resting and contracted cardiomyocytes. The threshold to determine buckling occurrence (horizontal line) was 2 standard deviations above the mean resting value. (FIG. 1I) Distribution of buckling wavelengths in cardiomyocytes shows a dominant population between 1.6 and 1.7 μm, and a second population at 3.3 μm. (FIG. 1J) A representative MT demonstrating buckles with wavelengths that correspond to the distance between 1 (1.65 μm) or 2 (3.3 μm) adjacent sarcomeres.

FIG. 2A-FIG. 2I. Detyrosination underlies microtubule buckling. (FIG. 2A) The MT cytoskeleton in rat adult cardiomyocytes (top) is heavily detyrosinated. TTL overexpression (bottom) reduces detyrosination dramatically but makes comparatively small changes in the overall MT network. (FIG. 2B) Quantification of the fraction of cell area covered by α- and dTyr-tubulin in null (n=14) and TTL overexpressing (n=13) cells as determined from thresholded images as shown in FIG. 9E. (FIG. 2C-FIG. 2D) Western blots from cardiomyocyte lysates show the effects of viral overexpression of TTL. (FIG. 2E) Time course of MTs in a contracting cardiomyocyte (cyan) transduced with AdV-TTL overlaid on the resting MT configuration (red). MTs appear to translocate along the contractile axis rather than deforming. (FIG. 2F) Comparison of MTs in resting (top) and contracted (bottom) cardiomyocytes in ctrl, TTL, and PTL groups. In TTL and PTL groups, some MTs slide relative to others that deform (arrows). Additional examples are provided in FIG. 10A-FIG. 10D. (FIG. 2G) Buckling occurrence and amplitude are reduced by overexpression of TTL or treatment with PTL. (FIG. 2H) Buckling wavelength distribution in ctrl and TTL overexpressing myocytes, and (FIG. 2I) the difference between these distributions. Overexpression of TTL causes MTs to buckle more often at wavelengths between 2-3 μm, and MTs are far less likely to buckle at distinct sarcomeric wavelengths (1.7 and 3.3 μm) when detyrosination is reduced. Data are presented as mean+/−SEM, *p<0.05; **p<0.01 ***p<0.001. Further statistical details are available in Table 1.

FIG. 3A-FIG. 3I. Detyrosinated MTs impede contractility. (FIG. 3A) Sarcomere shortening (ΔSL) during contraction is increased in TTL overexpressing myocytes. This change is dose dependent (FIG. 3B, p=1.2×10⁻⁵, r²=0.23) and associated with a faster shortening time without affecting resting sarcomere length (FIG. 3C). (FIG. 3D) First derivative of traces in (FIG. 3A) demonstrate contractile velocities in control and TTL overexpressing myocytes. TTL overexpressing myocytes demonstrate an increase in the peak velocity of both contraction and relaxation (FIG. 3E). PTL treated cells (FIG. 3F) display similar behavior. Despite the significant changes in contractility, no changes in the peak or kinetics of the global calcium transient were observable (FIG. 3G-FIG. 3I). Data are presented as mean+/−SEM, *p<0.05; **p<0.01 ***p<0.001. Further statistical details are available in Table 2.

FIG. 4A-FIG. 4E. Detyrosinated microtubules regulate the viscoelasticity of cardiomyocytes. (FIG. 4A) Elastic modulus of cardiomyocytes measured by AFM at various indentation velocities and fit to SLSM (see methods). (FIG. 4B) Quantification of velocity-independent (E1) and velocity-dependent (E2) components of the elastic modulus, and SLSM-fit derived viscosity. Both TTL overexpression and PTL treatment significantly reduced elasticity and viscosity. There were no significant differences in these parameters between DMSO and AdV-null transduced cells (p=0.28, 0.34, and 0.33, respectively). Reductions in stiffness due to TTL overexpression are also apparent in cells under stretch along the longitudinal axis. Myocytes were attached via glass cell holders (FIG. 4C, top) to a force transducer and length controller and subjected to stretch. MTs visualized by EMTB-3xGFP (FIG. 4C) at rest (top) and at a stretched length (bottom). (FIG. 4D) Representative force vs. length protocol. A series of stepwise stretches in 4 μm increments are applied to an isolated myocyte, increasing sarcomere length (SL). Passive tension generated by the step relaxes quickly from a peak value to a new steady state. (FIG. 4E) Force measurements binned according to measured change in sarcomere length with a given step size. TTL overexpressing cells exert reduced peak passive tension during step changes in length, with a more modest reduction in steady state tension. Data are presented as mean+/−SEM, *p<0.05; **p<0.01 ***p<0.001. Further statistical details are available in Tables 3 and 4.

FIG. 5A-FIG. 5H. Modeling microtubules in the contracting sarcomere. (FIG. 5A) Mechanical schematic of the modeled sarcomere. A force generating contractile arm (top) is coupled in parallel at the z-disc to a spring element representing titin, a viscoelastic medium, and a microtubule with anchors to the z-disc. The anchor to the Z-disc is only engaged at regions of MT detyrosination. TTL overexpression is modeled by allowing the anchors to slide for 50 nm at each end before engaging and transmitting force to the MT at a detyrosinated subunit (FIG. 5B). The change in sarcomere length (FIG. 5C) at peak contraction and buckling amplitude (FIG. 5C, FIG. 5D) recapitulate experimental observations for TTL overexpressing myocytes following this change. (FIG. 5E, FIG. 5F) The cardiac sarcomere shown with microtubules with putative stiff anchors to the sarcomere, shown here at the z-disc. Contraction reduces the distance between anchor points, requiring the MTs to either buckle (FIG. 5G) if the anchors are engaged or slide (FIG. 5H) if the anchors are not engaged and force incident on the MT remains low. Mathematical model parameters are available in Table 5.

FIG. 6A-FIG. 6G. Desmin associates with detyrosinated MTs to increase cardiomyocyte stiffness. (FIG. 6A) MT co-sedimentation shows the interaction between polymerized MTs (pellet) and desmin. (FIG. 6B) Quantification of the amount of detyrosination and desmin (relative to the total amount of tubulin) in the MT pellet from cardiomyocyte lysates with and without PTL treatment. Data normalized to DMSO level. (FIG. 6C) The amount of desmin associated with the MTs after PTL treatment is directly proportional to the amount of MT detyrosination across several experiments in rat cardiomyocytes and C2C12 cells. (FIG. 6D) Immunofluorescence of desmin, dTyr, and Tyr-tubulin shows dTyr-specific transverse pattern in WT but not desmin KO myocytes. (FIG. 6E) Overlay of detyrosinated-tubulin and desmin (see FIG. 14A-FIG. 14E for more examples). (FIG. 6F-FIG. 6G) AFM measurements show a PTL-dependent reduction in myocyte stiffness and viscosity in WT, but not desmin KO myocytes. Viscoelasticity in desmin KO myocytes is not statistically different from WT+PTL. Data are presented as mean+/−SEM, *p<0.05; **p<0.01 ***p<0.001 with respect to DMSO treatment; ###p<0.001 with respect to untreated WT myocytes. Further statistical details are available in Table 6.

FIG. 7A-FIG. 7H. Increasing detyrosination impairs contraction and is associated with human heart failure. (FIG. 7A) Western blot shows that shRNA against TTL selectively increases detyrosinated tubulin without changing overall levels of α-tubulin. (FIG. 7B) Elastic modulus of control and shTTL expressing myocytes at various indentation rates. (FIG. 7C) shTTL myocytes demonstrate increases in E1, E2 and viscosity. (FIG. 7D, FIG. 7E) TTL suppression significantly reduces contractile magnitude and velocity. (FIG. 7F) Representative western blots from human heart lysates. Data from pooled analysis is presented in (FIG. 7G). n=17 healthy donors, 9 hypertrophy, 17 DCM, 11 ischemic, 10 with DCM following ventricular assist device support (VAD DCM), and 15 HCM hearts. (FIG. 7H) There was a negative correlation between LVEF and detyrosinated tubulin expression in control and hypertrophic cardiomyopathy patients. Data are presented as mean+/−SEM, *p<0.05; **p<0.01 ***p<0.001. Further statistical details are available in Tables 7-9.

FIG. 8A-FIG. 8E. Principle of Airyscan imaging. (FIG. 8A) Confocal microscopes are typically optimized by setting the pinhole to coincide with the first intensity minimum of an airy disc to balance signal loss with resolution. A confocal microscope can exceed the diffraction limit by pinhole constriction (FIG. 8B), although at the cost of dramatic signal loss. Conversely, expansion of the pinhole (FIG. 8C) to allow more than 1 airy unit of light to pass can improve signal at the cost of resolution. Airyscan (FIG. 8D) uses a hexagonal array of 32 small aperture detectors (˜0.2 airy units) to map the point spread function over an area of ˜1.25 airy units in the confocal plane. The resulting 32 channels capture more total signal, but each maintains superior spatial resolution than is possible with a single airy unit pinhole. (FIG. 8E) Fluorescence intensity profile of confocal vs. airyscan across a single airy disc. Deconvolution of the resulting data can push resolution ˜1.7 fold below the theoretical limit for a 1 airy unit pinhole, while still maintaining the sensitivity to image at high speed.

FIG. 9A-FIG. 9F. (FIG. 9A) The microtubule cytoskeleton and detyrosinated fraction in control (top) and PTL treated (bottom) cardiomyocytes. Scale=10 μm. Networks were thresholded (FIG. 9B) and quantified (FIG. 9C) as in FIG. 2B-FIG. 2D. PTL does not have a significant effect on the overall microtubule network density but, like TTL overexpression, significantly reduces the detyrosinated fraction. (FIG. 9D) Western blotting shows a significant drop in overall detyrosinated tubulin, with no significant change in alpha tubulin levels following PTL treatment Thresholded images of the tubulin networks in Null (FIG. 9E) and TTL (FIG. 9F) overexpressing myocytes are provided for comparison.

FIG. 10A-FIG. 10D. (FIG. 10A) Cardiomyocyte microtubule cytoskeleton labeled with EMTB-3xGFP. Scale=10 μm. (FIG. 10B) Small regions at 3 different magnifications of labeled microtubules at rest (top) or buckling during contraction (bottom). Scale=2 μm. (FIG. 10C) Microtubule cytoskeleton of a TTL overexpressing cardiomyocyte. Overall morphology and density is similar to control. Scale=10 μm. (FIG. 10D) Small regions at 3 different magnifications of microtubules at rest (top) and during contraction (bottom). Scale=2 μm. Note the low incidence of buckling in FIG. 10D when compared with FIG. 10B.

FIG. 11A-FIG. 11G. Representative force-displacement curves from AFM studies: (FIG. 11A-FIG. 11C) Force calculated from cantilever deflection and spring constant is plotted vs. Z-piezo position (Ind) and the resulting curves are fit to the Hertz equation for a spherical indenter (cf. methods) to determine the elastic modulus of the myocyte. The shape of the indentation is well fit to the Hertz equation for all indentation velocities. The upper traces show the measured force during the approach of the cantilever towards the cell, the Hertz equation is fit to this curve, and the lower traces show the retraction force. The negative force in the lower trace represents adhesion of the cardiomyocyte to the AFM probe during retraction that causes downward deflection of the cantilever. Strong adhesion is an issue for using the Hertz equation if long-range attractive or repulsive forces are experienced during the indentation leading to cantilever deflection before the AFM probe contacts the cell. The flatness of the upper approach trace before initial contact indicates that the adhesive forces between the AFM probe and the cardiomyocyte are short-range, and thus the Hertz model is justified for fitting the data. Further justification and description of the technique is provided in (43). In (FIG. 11D) the dependence of elastic modulus on indentation depth is shown. Even at large indentation depths (relative to the radius of the indenter) beyond that used in these experiments, the elastic modulus is relatively insensitive to the depth of indentation. (FIG. 11E-FIG. 11F) show control data fit to different models of viscoelasticity. The standard linear solid model is sufficient to capture the shape of the data over the velocity range probed in this experiment (FIG. 11E), while the Kelvin-Voight model cannot (FIG. 11F). Adding a dashpot in parallel with the standard linear solid model (FIG. 11G) is also sufficient in capturing the shape of the data, and enables the elastic modulus to continue to trend upwards at large velocities, but does not significantly improve the model over the frequency range tested.

FIG. 12A-FIG. 12G. (FIG. 12A) 3D-reconstruction from confocal z-stack of an EMTB-3xGFP decorated cardiomyocyte (cyan) attached to MyoTak-coated cell holders (orange). One cell holder is connected to an optical force transducer, the other to a piezoelectric length controller. (FIG. 12B) The orthogonal view of the cell in the holder. (FIG. 12C-FIG. 12E) 3D reconstructions of the cell in FIG. 12A under stretch (FIG. 12C), at resting length (FIG. 12D) and compressed (FIG. 12E). Panels on the right are magnifications of the outlined region of the cell. (FIG. 12F) Force vs. piezo step length. (FIG. 12G) Change in length vs. change in sarcomere length. Note that the TTL overexpressing myocytes are significantly more compliant than the Null group.

FIG. 13A-FIG. 13G. Microtubules and the contracting sarcomere. (FIG. 13A) The cardiac sarcomere consists of repeating units of actin filaments anchored to the z-disc and myosin (which are spaced at resting length by titin. The viscoelastic elements of the cytoplasm, including other microtubules and cytoskeletal elements, surround and laterally brace microtubules which are presumed to have relatively rigid anchors to the sarcomere. During contraction (FIG. 13B), opposing arrays of actin are pulled together by myosin, reducing the distance between anchor points. This results in buckling of the microtubule and the force required to deform a microtubule provides moderate resistance to contraction. (FIG. 13C) A cardiomyocyte under stretch should pull anchored microtubules taut, providing some anchor-dependent resistance to sarcomere stretch. This may underlie the altered step length to sarcomere length relationship noted in FIG. 4D. The right side of the model displays potential scenarios that could alter MT buckling. If the rigidity of the microtubules is reduced (FIG. 13D), the reduced microtubule to medium stiffness ratio should promote shorter wavelength buckles that are easier to form. If the viscoelastic stiffness of the cytoplasm is reduced (FIG. 13E), the less constrained microtubules should again buckle more readily, and also adopt a longer wavelength and higher amplitude. If microtubules are uncoupled from the sarcomeres (FIG. 13F) however, the reduced incident force makes microtubule deformation unlikely, while any microtubules that maintain anchors to multiple sarcomeres buckle under similar but more broadly distributed parameters. FIG. 13F matches our experimental observations when MT detyrosination is reduced with PTL and TTL (FIG. 2G-FIG. 2H). Disrupting MT cross-linking to sarcomeres would likewise result in decreased compression resistance in both the transverse and longitudinal axis, reducing the stiffness of the myocyte. (FIG. 13G) Model predictions for changes in MT rigidity, medium viscoelasticity, and coupling to sarcomeres (changing incident force). Of these three factors that can alter MT buckling, only a decrease in incident force predicts all 3 major experimental observations for sarcomere contractility, buckling wavelength, and buckle amplitude in TTL overexpressing myocytes.

FIG. 14A-FIG. 14H. Immunofluoresence of desmin, and microtubules. (FIG. 14A) In rat or WT mouse myocytes, desmin occurs primarily in transverse bands at the z-disk. In stains for detyrosinated tubulin, a faint sarcomeric pattern emerges which is absent in the tyrosinated stain. (FIG. 14G) This pattern remains in myocytes where no labeling of desmin occurs and with different secondary antibodies, indicating that the pattern is not due to cross reactivity. Desmin knockout (FIG. 14B), however, eliminates this pattern entirely and causes the overall MT network to adopt a denser and more chaotic structure. (FIG. 14C) This effect is specific to desmin and not due to disruption of the z-disk, as alpha-actinin maintains its sarcomeric banded pattern, although detyrosinated tubulin no-longer preferentially localizes there. (FIG. 14D) These transverse structures can be appreciated to a lesser extent using a pan α-tubulin antibody. (FIG. 14F) Quantification shows that desmin KO significantly increased cell area covered by MTs, indicative of a denser network. (FIG. 14H) Blind scoring of unlabeled immunofluorescence images demonstrates that the sarcomeric pattern is only definitively identified in dTyr labeling of WT myocytes, and that this pattern is not identified in desmin KO myocytes (n=31 images of WT-dTyr, 31 WT-tyr, 7 WT-pan tubulin, 9 KO-tyr, 9 KO-dTyr, and 7 KO pan tubulin images)

FIG. 15A-FIG. 15B. Enhancement of detyrosination by shRNA against TTL. (FIG. 15A) Representative myocyte immunostained for detyrosinated tubulin. (FIG. 15B) Quantification of increase in detyrosinated MT density and intensity with shTTL.

FIG. 16A-FIG. 16E. Correlations between contractility and tubulin expression in human patient samples. Each graph demonstrates the relationship between left ventricular ejection fraction (LVEF) and tubulin expression (alpha, detyr, or tyr) in left ventricular tissue samples from human patients. Data is fit to a linear function and the correlation tested for significance via ANOVA. No significant correlations were found for any tubulin sub-type in dilated cardiomyopathy groups (DCM, Ischemia, PostVAD DCM).

FIG. 17. The buckled shape of the MT with initial length L₀. u(x) is the transverse displacement of each point. The amplitude and wavelength of the buckled shape are denoted by A and λ.

FIG. 18. The buckling amplitude of the MT (with initial length L₀=1.8 μm) with time under a constant force (F=360 pN). Time-scale for the relaxation of the medium is set to η=0.1 sec.

FIG. 19. Schematic map of the adenoviral plasmid carrying the human cytomegalovirus promoter, attB1, a T7 promoter, the TTL coding sequence (cds), an IRES linker, an attB2 sequence, a V5 epitope, and a TK polyA signal in a human adenovirus 5 backbone. The attB1 and attB2 facilitate expression of the TTL enzyme as a fusion protein containing the V5 epitope [GKPIPNPLLGLDST] (SEQ ID NO: 9) as a tag peptide which allows monitoring expression for the studies described herein.

FIG. 20A-FIG. 20D. Proteomic analysis of human left ventricular tissues of varying disease severity and etiology. (FIG. 20A) Principal component analysis (PCA) of tandem MS data (500 most variable proteins) to visualize similarities and differences among samples. Small circles represent the projections of individual hearts onto principal component (PC) 1 and 2, with the percentage of total variance listed in parentheses; large circles and ellipses represent the group mean and 95% confidence intervals, respectively. Non-failing (normal and cHyp), ischemic, and non-ischemic heart failure display unique proteomic profiles (Normal N=7, cHyp N=6, ICM N=6, HCMpEF N=4, HCMrEF N=5, DCM N=6). (FIG. 20B) Heat map displaying the molecular function GO groups enriched in each group when compared to normal, color-coded by significance and sorted by most increased in DCM. Cytoskeleton-related GO groups are highlighted (i.e., structural constituent of cytoskeleton, actin binding, and structural constituent of muscle) and represent the dominant change in DCM and HCMrEF. (FIG. 20C) Heat map depicting the expression levels (log₂ fold change) of individual proteins in the major cytoskeletal sub-groups. IFs and the major tubulin isoforms are increased, while there is a general decline in muscle actin and myosin motors. NF, non-failing. (FIG. 20D) Top left—top 10 upregulated genes as obtained from differential gene expression analysis in HCMrEF and DCM (compared to normal). Cytoskeletal genes are highlighted (i.e., SVIL, DES, SYNM, MXRA7, THBS4, NES, and MAP4). Box plots show abundance of specific proteins of interest measured as LFQ value from MS data. Each data point represents one heart, with mean line and whiskers representing standard deviation (SD). Statistical significance was determined via differential gene expression analysis, in which a linear model adjusting for age and sex in the R package LIMMA was used. P values were adjusted for multiple testing using the Benjamini-Hochberg procedure, *p<0.05, **p<0.01, ***p<0.001.

FIG. 21A-FIG. 21F. Proteomic analysis of human left ventricular tissues. (FIG. 21A) Box plot of log 2 label free quantification (LFQ) values (MS signal). Medians of log 2 signal are similar across all samples, suggesting even sample loading in MS and no need for data normalization. (FIG. 21B) PCA plot of the whole proteome shows similar clustering of samples compared to FIG. 20A. (FIG. 21C) 2D-PCA biplot with 200 most variable proteins input and 50 proteins output. Intermediate filament proteins desmin (DES), synemin (SYMN), vimentin (VIM) and nestin (NES) are among the top 50 output proteins contributing to the distribution. (FIG. 21D, FIG. 21E) In the same PCA plot as shown in FIG. 20A with 500 proteins input, samples are grouped by different factors: sex (FIG. 21D, female in pink and male in teal) and age (FIG. 21E). Note that samples are sparsely clustered and overlapping between/among groups, indicating no biased distribution based on sex or age. (FIG. 21F) Similar distribution of age in patients with different etiology in this study. Hearts used in mass spectrometry are highlighted in red and hearts used in single cell functional studies are highlighted in blue. All hearts (black/red/blue) were subject to biochemical analysis.

FIG. 22A-FIG. 22F. Characterization of MTs and desmin in NF and failing human myocytes. (FIG. 22A) Immunofluorescence images of surface and interior MTs and desmin in a failing human myocyte. (FIG. 22B) Structured illumination microscopy (SIM) of NF human myocytes. Top, MTs. Bottom, a higher magnification image of transverse desmin elements and longitudinal MTs. (FIG. 22C) Representative dTyr- and Tyr-MT networks in NF and failing myocytes. (FIG. 22D) Top, immunofluorescence images were converted to binary images to quantify MT network density. The total MT network density was calculated from the overlay of dTyr-MT and Tyr-MT binary images (scale bar, 2 μm). Bottom, the percentages of cell area covered by polymerized MTs (left) and the ratio of dTyr-MT/total MT (right) (NF, n=68 cells from N=3 hearts; failing, n=42 cells from N=2 hearts). Statistical significance in FIG. 22D determined via two-sided T-test with post-hoc Bonferroni correction, ***p<0.001 vs. NF. (FIG. 22E, 22F) Representative western blot of desmin (FIG. 22E) and quantification of the levels of α-tubulin and the 53 kD and low MW forms of desmin (FIG. 22F). GAPDH was used as a loading control. Each dot represents individual heart with mean line and SD (Normal N=24, cHyp N=18, HCM N=19, DCM N=26, ICM N=15 hearts). Statistical significance in FIG. 22F determined via two-sided T-test with post-hoc Bonferroni correction, *p<0.05, **p<0.01, ***p<0.001 vs. normal. A representative α-tubulin blot and additional image quantification of MTs and desmin in myocytes and myocardium are presented in FIG. 23A-23E and FIG. 24A-24C.

FIG. 23A-FIG. 23E. Quantification of desmin periodicity and non-myocyte area in human heart failure. (FIG. 23A) Representative western blot of α-tubulin (quantification shown in FIG. 22F). (FIG. 23B) Immunofluorescence of α-tubulin and desmin on human left ventricular tissue sections. Upper left image shows fragmented MTs during the fixation of myocardial tissue. (FIG. 23C) Quantification of desmin_{power} from Fast Fourier Transform (FFT) analysis of tissue sections. Relative desmin_{power} was calculated using power spectrum obtained from 2D FFT. Desmin_{power} is significantly reduced in HCM, DCM and ICM, suggesting loss of desmin periodic organization in heart failure. (FIG. 23D) Quantification of non-myocyte area in tissue section immunofluorescence images. Non-myocytes occupy a small fraction of myocardium relative to myocytes and it is modestly increased in HCM and ICM but not in DCM. (FIG. 23E) SIM image of desmin immunofluorescence in isolated human myocytes. Note that failing myocyte displays desmin disorganization and myofibril streaming, indicating that myofiber alignment is disrupted in heart failure. Statistical significance determined via ANOVA with post-hoc Tukey test, *p<0.05, ***p<0.001 vs. normal; ###p<0.001 vs. cHyp.

FIG. 24A-FIG. 24C. MT directionality analysis. (FIG. 24A) Representative image of surface and interior MT network in NF and failing myocyte. (FIG. 24B, 24C) MT directionality analysis. Overall, MT network organization is not grossly disrupted in failing myocytes, but the proportion of longitudinal vs. transverse MTs is slightly higher than in NF myocytes.

FIG. 25A-FIG. 25C. MT-dependent viscoelasticity of human myocytes measured via nanoindentation. (FIG. 25A) Viscoelasticity data are plotted as stiffness (elastic modulus) vs. velocity of indentation and is pooled from cardiomyocytes from NF and failing hearts treated with DMSO, colchicine (colch, a MT depolymerizer) or PTL (inhibits detyrosination) (NF hearts: DMSO, N=6 hearts, n=34 cardiomyocytes; colch, N=5, n=26; PTL, N=3, n=16; Failing hearts: DMSO, N=5, n=26; colch, N=5, n=30; PTL, N=2, n=9). (FIG. 25B) Quantification of viscoelasticity measurements Eₘᵢₙ (minimum stiffness at low rate), Eₘₐₓ (maximum stiffness at high rate), and EΔ (difference between Eₘᵢₙ and Eₘₐₓ an indicator of viscoelasticity). Statistical significance determined via ANOVA with post-hoc Tukey test, ***p<0.001 vs. DMSO; ##p<0.01, ###p<0.001 vs. NF. (FIG. 25C) Correlation between the initial viscoelasticity (EΔ) of each heart and the percentage decreases in viscoelasticity after colch (square) and PTL (circle) treatment Each data point represents the mean EΔ from all cells in a particular heart, which are color coded by group.

FIG. 26A-FIG. 26G. Suppression of detyrosinated MTs improves contractility in failing human cardiomyocytes. (FIG. 26A) Average sarcomere shortening from DMSO-treated myocytes from failing and non-failing hearts. Shortening is shown normalized to resting length; the negative deflection represents contraction in response to electrical stimulation followed by relaxation back to the resting length. (FIGS. 26B and 26C) Effects of colchicine or PTL on the contractility of myocytes from representative NF (FIG. 26B) and failing (FIG. 26C) hearts. (FIG. 26D) Average velocity traces from all NF, failing, and treated failing myocytes. (FIG. 26E) Correlation between initial velocity in untreated myocytes and percentage improvement in velocity after treatment with colchicine (square) or PTL (circle). Each data point represents the mean contraction and relaxation velocity from all cells in a particular heart color coded by group. (FIGS. 26F and 26G) Pooled data showing percentage improvement in the indicated contractile parameters following treatment of NF or failing hearts with colch (FIG. 26F) or PTL (FIG. 26G), as compared to vehicle-treated cells. Boxes show 25^{th}-75^{th} percentile, with median notch and mean line. Relax, relaxation; SL, sarcomere length. Statistical significance determined via two-sided T tests, *p<0.05, **p<0.01, ***p<0.001 vs. DMSO; #p<0.05, ##p<0.01, ###p<0.001 vs. NF.

FIG. 27A-FIG. 27H. Neither colchicine nor PTL rescue [Ca²]ᵢ transient in failing myocytes. [Ca²⁺]ᵢ transients in electrically stimulated human myocytes loaded with Ca2⁺ indicator fluo3. Data is presented as the fold change of fluo3 intensity (F) relative to the baseline intensity (F0). (FIG. 27A) Failing myocytes show lower amplitude of [Ca²⁺]ᵢ transients during contraction, suggesting a defect in EC coupling in failing hearts. (FIG. 27B) [Ca²⁺]ᵢ in NF myocytes with or without MT destabilization. (FIGS. 27C and 27D) Normalized [Ca²⁺]ᵢ transients in NF and failing myocytes. (FIG. 27E-27G) Quantification of [Ca²⁺]ᵢ amplitude and kinetics. White parenthesis indicates number of cells. On average, myocytes treated with colchicine show no changes in [Ca²⁺]ᵢ amplitude or kinetics, whereas PTL-treated NF cells have reduced [Ca²⁺]ᵢ amplitude and slower [Ca²⁺]ᵢ decay (FIG. 27G) suggesting an off-target effect of PTL. (FIG. 27H) Normalized intensity traces in different treatments. PTL treated myocytes had elevated F0 compared to their initial levels, contributing to the lower F/F0 in PTL treated myocytes when the last five steady transients are analyzed. Statistical significance determined via ANOVA with post-hoc Tukey test, *p<0.05, ***p<0.001 vs. NF DMSO, # p<0.05, ###p<0.001 vs. NF Colchicine; ∧∧∧ p<0.001 vs. NF PTL.

FIG. 28A-FIG. 28F. Genetic modification of tubulin tyrosination reduces stiffness and improves contractility. (FIG. 28A) Quantification of MT network density and dTyr-MT/total MT ratio (as described in FIG. 2d) following adenoviral-mediated overexpression of TTL (TTL) in cultured human cardiomyocytes, compared to myocytes infected with a null encoding adenovirus (Null). (FIG. 28B) Average trace of sarcomere shortening in AdV-Null and AdV-TTL overexpressing cardiomyocytes. (FIG. 28C) Average contractile velocities of AdV-Null and AdV-TTL overexpressing cardiomyocytes. (FIG. 28D) Quantification of contractile parameters. Each data point represents a single cardiomyocyte infected with AdV-null (n=80) or AdV-TTL (n=77) from N=3 hearts. (FIG. 28E) Viscoelasticity data and (FIG. 28F) quantification of viscoelasticity measurements on AdV-TTL and AdV-null expressing myocytes, presented and quantified as described in FIG. 25A-25C. Statistical significance determined via two-sided T-test with post-hoc Bonferroni correction, ***p<0.001 vs. AdV-null.

FIG. 29A-FIG. 29C. Improved contractile kinetics upon TTL overexpression is preserved in both 0.5 Hz and 1 Hz contractions at 37° C. (FIGS. 29A and 29B) Representative traces of 0.5 Hz (FIG. 29A) and 1 Hz (FIG. 29B) contractions in myocytes from a NF heart at 37° C. Of note, contraction kinetics and amplitude are similar in AdV-null cells paced at 1 Hz and 0.5 Hz. (FIG. 29C) Quantification of percentage improvement with TTL overexpression on shortening, contraction velocity and kinetics from two hearts (1 NF and 1 failing). Suppressing detyrosinated MTs with TTL-overexpression still significantly improved contractile amplitudes as well as contraction and relaxation kinetics at physiological temperature and pacing frequency. Further, the magnitude of improvement was similar in 0.5 Hz and 1 Hz contractions. Statistical significance determined via two-sided T-tests, *p<0.05, **p<0.01, ***p<0.001 vs. AdV-null.

FIG. 30A-FIG. 30D. EpoY, a new small molecule inhibitor of the detyrosinating enzyme reduces detyrosination and improves cardiomyocyte contractility. (FIG. 30A) Concentration and time dependence of detyrosinated-tubulin expression in rat ventricular myocytes treated with EpoY. Short term EpoY treatment specifically reduces detyrosination without changing total tubulin content, lowering the dTyr/Tyr ratio. Based on these results, 2 hrs of 10 μM EpoY was used for all subsequent functional experiments. (FIG. 30B) Electrically evoked [Ca²⁺]ᵢ transients are not changed with EpoY treatment. (FIG. 30C and FIG. 30D) In contrast, contractility is enhanced by EpoY treatment, with an increase in the amplitude of contraction (FIG. 30C) and the velocity of both contraction and relaxation (FIG. 30D).

FIG. 31A-FIG. 31F. Chronic TTL overexpression lowers stiffness and improves contractility in murine myocytes. 3 P5 rats were injected pericardially with an adeno-associated virus encoding TTL and mCherry driven by a cardiomyocyte specific promoter (AAV9-cTnT-TTL-mCherry). Myocytes were isolated from adult rats 8 weeks later. Transduction efficiency was ˜40%, allowing internal, non-mCherry expressing controls. After cellular assays were performed cells were binned based on mCherry fluorescence, a readout of TTL expression. (FIG. 31A and FIG. 31B) Electrically stimulated Ca²⁺ transients were not different with TTL overexpression. (FIG. 31C and FIG. 31D) In contrast, contractility was augmented. Shortening amplitude, contraction velocity, and relaxation velocity were all significantly increased in cells with high TTL expression. (FIG. 31E) Stiffness (Elastic Modulus at 10 um/s indentation) was reduced in TTL overexpressing myocytes. (FIG. 31F) Western blot quantification (vs. 3 uninfected littermates) from left ventricular tissue lysate shows that despite only 40% transduction efficiency, dTyr/Tyr ratio is significantly reduced with AAV-TTL.

FIG. 32. Comparison of the effects of various pharmacological and genetic approaches on contraction velocity, relaxation velocity, and contraction amplitude in primary adult rat cardiomyocytes. Data shown are normalized as a fold change relative to the appropriate control for that treatment group (DMSO treatment for pharmacological agents, a null-encoding adenovirus for adenoviral over expression of TTL (TTL O.E.), and a scramble shRNA-encoding adenovirus for adenoviral knock down of TTL with shRNA (TTL K.D.)). Vinblastine was used at 20 μM (diluted in DMSO) and applied throughout 2 hr incubation of cells. Nocadazole was used at 1 μM (diluted DMSO) and applied throughout 30 min incubation of cells. Costunolide, EpoY, and PTL were used at 10 μM (DMSO) and applied throughout 2 hr incubation of cells.

### DETAILED DESCRIPTION OF THE INVENTION

Provided herein is a method for improving heart function and/or treating heart failure. In one embodiment, a method for treating heart failure in humans comprises delivering a composition comprising a therapeutic which interferes with detyrosination of microtubules in cardiomyocytes. In certain embodiments, a method for improving heart function in humans is described. This method comprises delivering a composition comprising a therapeutic which increases cardiac microtubule tyrosination.

### Inhibitors of Tubulin Carboxypeptidase and Modulation of Detyrosination

In one aspect, a method for improving heart function in humans is provided which comprises treating a patient with a therapeutic which inhibits tubulin carboxypeptidase (TCP). In certain embodiments, the therapeutic (active ingredient) may be sesquiterpene lactones, such as parthenolide or costunolide, or a prodrug, derivative, pharmaceutically acceptable salt or analog thereof. In yet another embodiment, the therapeutic is an inhibitor of TCP activity such as epoY, epoEY, or epoEEY.

In another aspect, a method for treating heart failure in humans is provided which comprises dosing a patient with a therapeutic which interferes with detyrosinated microtubules in cardiomyocytes. The therapeutic may be a small molecule drug selected from one or more of: sesquiterpene lactones including parthenolide (PTL), costunolide or PTL pro-drugs such as LC-1, or microtubule destabilizers including colchicine, vinblastine, and nocodazole. In certain embodiments, a method is provided for treating patients with a composition which decreases detyrosination of cardiac microtubules. This method is useful for stabilizing loss of heart function and/or preventing heart failure in patients (e.g., humans). The therapeutic may be a small molecule drug selected from one or more of: sesquiterpene lactones including parthenolide (PTL), costunolide or PTL pro-drugs such as LC-1, or microtubule destabilizers including colchicine, vinblastine, and nocodazole. In yet another embodiment, the therapeutic is an inhibitor of TCP activity such as epoY, epoEY, or epoEEY. In certain embodiments, a method is provided for treating patients with a composition which decreases or prevents detyrosination of cardiac microtubules.

Optionally, one or more of these drugs is delivered to the patient's heart alone, or as a co-therapeutic. Optionally, the therapy may involve co-administration with one or more drugs, and/or one or more of the compositions described herein.

As used herein, the term “active sesquiterpene lactone” refers to a sesquiterpene lactone that has an α-methylene-γ-lactone functional group, and that is capable of inhibiting or reducing tubulin carboxypeptidase (TCP) and/or which interferes with detyrosinated microtubules in cardiomyocytes. In certain embodiments, a dose of an active sesquiterpene lactone or another selected compound described herein is in the range of about 0.001 mg to about 1000 mg per dose, and values therebetween. In other embodiments, a dose is in the range of about 10 mg to about 500 mg, or about 20 mg to about 50 mg. In other embodiments, the dose is 0.01 μg/kg body weight to about 500 mg/kg body weight, or about 1 mg/kg body weight to about 10 mg/kg body weight.

Examples of an active sesquiterpene lactone may be, without limitation one or more of encelin, parthenolide, leucanthin B, enhydrin, melampodin A, tenulin, confertiflorin, burrodin, psilostachyin A, costunolide, costinulide, and/or cinerenin, or another compound provided herein. See, e.g., U.S. Pat. No. 5,590,089. Such active sesquiterpene lactones may be used in various combinations or mixtures. In addition prodrugs, derivatives, pharmaceutically acceptable salts, and solvates thereof are useful in the compositions and methods described herein. Further, other active sesquiterpene lactones may be selected.

“Parthenolide” refers to a compound having the structure:

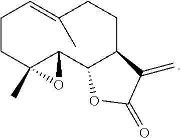 [SMILES: C=C1C(=O)O[C@H]2[C@H]1CC/C(C)=C/CC[C@@]1(C)O[C@@H]21]

Parthenolide is sesquiterpene lactone and a member of the germacranolide class. [4,5-epoxygermacra-1(10),11(13)-dien-12,6-olactone]. In addition to parthenolide, prodrugs, derivatives, pharmaceutically acceptable salts, and solvates thereof are useful in the compositions and methods described herein. Further, other active sesquiterpene lactones may be selected. Optionally, one or more of these compounds may be used in combinations with one or more active compounds.

“Costunolide” refers to a compound having the structure:

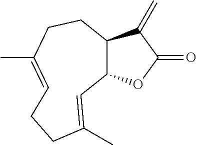 [SMILES: C=C1C(=O)O[C@@H]2/C=C(\C)CC/C=C(\C)CC[C@@H]12]

Costunolide, (3aS,6E,10E,11aR)-6,10-dimethyl-3-methylidene-3a,4,5,8,9,11a-hexahydrocyclodeca[b]furan-2-one, is a sesquiterpene lactone and member of the germacranolide class (see, e.g, compound summary for CID 5281437 in the PubChem database available online at https://pubchem.ncbi.nlm.nih.gov/compound/Costunolide). Costunolide can be naturally occurring or prepared synthetically and may be identified by other names such as (+)-costunolide, costunlide, costundide, costunolid, and costinulide. Compounds suitable for use in compositions and methods described herein are commercially available from various sources (e.g., Sigma-Aldrich, SML0417). In addition to costunolide, prodrugs, derivatives, pharmaceutically acceptable salts, and solvates thereof are useful in the compositions and methods described herein. Further, other active sesquiterpene lactones, as well as prodrugs, derivatives, pharmaceutically acceptable salts, and solvates thereof, may be selected. These compounds, or pharmaceutically acceptable salts, solvates, or derivates thereof, may be used as described herein. Optionally, one or more of these compounds may be used in combinations with one or more active compounds.

Additional examples of active sesquiterpene lactones (including, e.g., parthenolide analogues or derivatives) include those provided in U.S. Pat. No. 9,266,901, which is hereby incorporated by reference herein. Examples of such compounds may include those having the structure of Formula I, II, III, or IV, which follow, or a pharmaceutically acceptable salt, prodrug, enantiomer, or solvate thereof:

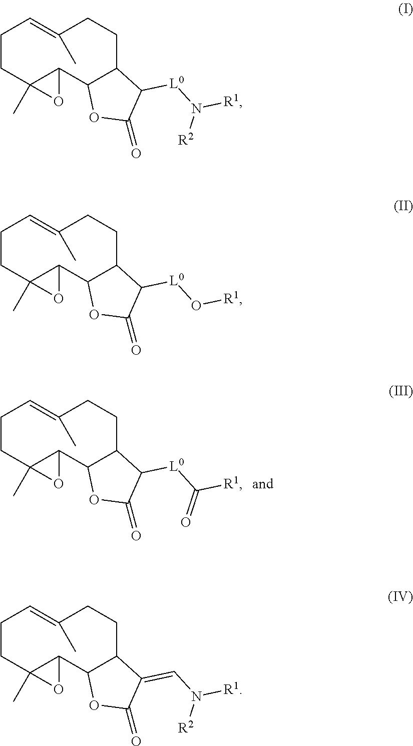

L⁰ is independently a bond or an unsubstituted C1-C10 alkylene. R¹ and R² are independently hydrogen, —OH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; where R¹ and R² may optionally be joined to form a substituted or unsubstituted heterocycloalkyl or a substituted or unsubstituted heteroaryl. The definitions of these substituents from U.S. Pat. No. 9,266,901 are incorporated by reference.

In certain embodiments, epoY, epoEY, or epoEEY, may be selected for use in a method described herein. EpoY, epoEY, and epoEEY contain the epoxide functional group from parthenolide coupled to one, two, or three amino acids from the α-tubulin C terminus, respectively. (Single-letter abbreviations for the amino acid residues are as follows: A, Ala; C, Cys; D, Asp; E, Glu; F, Phe; G, Gly; H, His; I, Ile; K, Lys; L, Leu; M, Met; N, Asn; P, Pro; Q, Gln; R, Arg; S, Ser; T, Thr; V, Val; W, Trp; and Y, Tyr. See, e.g., Aillaud et al, Science 358 (6369), 1448-1453 and the supplementary materials therewith, incorporated by reference in its entirety, for an illustrative description of synthesis of these compounds.

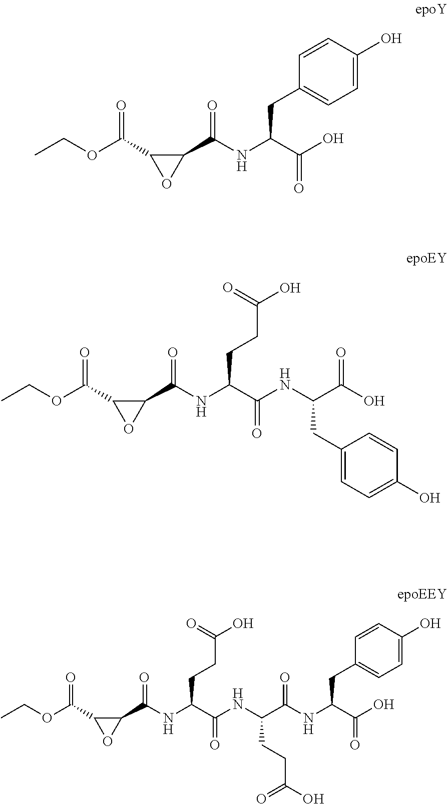

These compounds, or pharmaceutically acceptable salts, solvates, or derivates thereof, may be used as described herein. Optionally, one or more of these compounds may be used in combinations with one or more active compounds.

The term “pharmaceutically acceptable salts” includes salts of the active compounds that are prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When the compounds contain relatively basic functionalities, acid addition salts can be obtained by contacting the neutral form of such compounds with a sufficient amount of the desired acid, either neat or in a suitable inert solvent Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolyl-sulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66: 1-19 (1977)). Certain specific compounds contain both basic and acidic functionalities that allow the compounds to be converted into either base or acid addition salts. Other pharmaceutically acceptable carriers known to those of skill in the art are suitable. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

“Pharmaceutically acceptable excipient” and “pharmaceutically acceptable carrier” refer to a substance that aids the administration of an active agent to and absorption by a subject and can be included in the compositions described herein without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution alcohols, oils, gelatins, carbohydrates such as lactose, amylase or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the active compounds. One of skill in the art will recognize that other pharmaceutical excipients are useful.

The term “preparation” is intended to include the formulation of the active compound with encapsulating material as a carrier providing a capsule in which the active component with or without other carriers, is surrounded by a carrier, which is thus in association with it. Similarly, cachets and 40 lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject Administration is by any route, including parenteral and transmucosal (e.g., buccal, sub lingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. biologic or viral vector).

In other embodiments, a therapeutic may be selected which interferes with detyrosinated cardiac microtubules. One or more of the active sesquiterpene lactones may be used. However, in certain embodiments, a colchicine is selected. This compound has the chemical name: (S)—N-(5,6,7,9-tetrahydro-1,2,3,10-tetramethoxy-9 oxobenzol[a]heptalen-7-yl) acetamide and the structural formula:

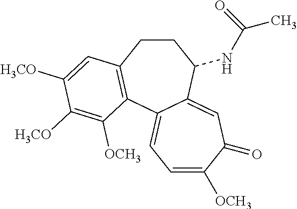 [SMILES: COc1cc2c(c(OC)c1OC)-c1ccc(OC)c(=O)cc1[C@@H](NC(C)=O)CC2]

This compound is commercially available under the brand name Colcrys®. It will be understood that a pharmaceutically acceptable salt of this compound, or a prodrug, solvate, or enantiomer thereof, may be selected. With respect to colchicine a particularly preferred range is an amount of from 0.6 mg/day to about 1.2 mg/day but the dose may be varied, e.g., in a range from 0.3 or 0.6 mg/day to 1.8 or 2.4 mg/day.

One or more of the compounds identified herein may be administered alone or can be co-administered in a combination with one or more active compounds to the patient. Co-administration is meant to include simultaneous or sequential administration of the compound individually or in combination (more than one compound or agent). Thus, the preparations can also be combined, when desired, with other active substances. The compositions provided herein can be delivered by transdermally, by a topical route, formulated as applicator sticks, solutions, suspensions, emulsions, gels, creams, ointments, pastes, jellies, paints, powders, and aerosols. Oral preparations include tablets, pills, powder, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions may additionally include components to provide sustained release and/or comfort. Such components include high molecular weight, anionic mucomimetic polymers, gelling polysaccharides and finely-divided drug carrier substrates. The compositions can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1 995; as biodegradable and injectable gel formulations (see, e.g., Gao Pharm. Res. 12: 857-863, 1 995); or, as micro spheres for oral administration (see, e.g., Eyles, J. Pharm. Pharmacol. 49: 669-674, 1997). In another embodiment, the formulations of the compositions can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ (e.g., the heart), one can focus the delivery of the compositions into the target cells in vivo.

### Increase Tyrosination

In certain embodiments, a method is provided for treating patients with a composition which increases tyrosination in the cardiac microtubule tyrosination. This method is useful for improving heart function in patients (e.g., humans) for improving heart function and/or for preventing heart failure in patients in need thereof. This method involves delivering tubulin tyrosine ligase (TTL) to the patient. Optionally, the enzyme may be administered delivered to the patient's heart alone, or as a co-therapeutic. Desirably, however, the enzyme is expressed from a nucleic acid molecule delivered to the patient. In certain embodiments, the nucleic acid molecule is specifically targeted to the heart. In still other embodiments, the nucleic acid molecule is specifically targeted to the cardiac microtubules. The nucleic acid may be delivered by non-viral delivery systems and/or by viral delivery systems. Optionally, the therapy may involve co-administration of two or more of the enzyme, a nucleic acid expressing the enzyme, and/or a small molecule drug which reduces detyrosination and/or inflammation.

### Tubulin-Tyrosine Ligase (TTL) Expression Cassette

As used herein, the term “tubulin-tyrosine ligase” refers to a human enzyme which catalyzes the post-translational addition of a tyrosine to the C-terminal end of detyrosinated alpha-tubulin. One suitable human amino acid sequence is provided in UNIPROT [Q8NG68] (377 amino acids in length), available at: www.uniprotorg/uniprot/Q8NG68:

(SEQ ID NO: 5)
MYTFVVRDENSSVYAEVSRLLLATGHWKRLRRDNPRFNLMLGERNRLPFG
RLGHEPGLVQLVNYYRGADKLCRKASLVKLIKTSPELAESCTWFPESYVI
YPTNLKTPVAPAQNGIQPPISNSRTDEREFFLASYNRKKEDGEGNVWIAK
SSAGAKGEGILISSEASELLDFIDNQGQVHVIQKYLEHPLLLEPGHRKFD
IRSWVLVDHQYNIYLYREGVLRTASEPYHVDNFQDKTCHLTNHCIQKEYS
KNYGKYEEGNEMFFKEFNQYLTSALNITLESSILLQIKHIIRNCLLSVEP
AISTKHLPYQSFQLFGFDFMVDEELKVWLIEVNGAPACAQKLYAELCQGI
VDIAISSVFPPPDVEQPQTQPAAFIKL.

Any suitable coding sequence for this protein may be backtranslated, optionally taking into consideration the codons preferred for human use. Such a nucleic acid sequence may be DNA (e.g., cDNA) or RNA (e.g., mRNA, tRNA, among others).

In one embodiment, a defective vector is provided which comprises a nucleic acid sequence encoding tubulin tyrosine ligase (TTL) under the control of a regulatory control sequence which directs expression thereof in the heart.

As used herein, the term “operably linked” refers to both expression control sequences that are contiguous with the gene of interest and expression control sequences that act in trans or at a distance to control the gene of interest.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

Identity or similarity with respect to a sequence is defined herein as the percentage of amino acid residues in the candidate sequence that are identical (i.e., same residue) or similar (i.e., amino acid residue from the same group based on common side-chain properties, see below) with the peptide and polypeptide regions provided herein, after aligning the sequences and introducing gaps, if necessary, to achieve the maximum percent sequence identity. Percent (%) identity is a measure of the relationship between two polynucleotides or two polypeptides, as determined by comparing their nucleotide or amino acid sequences, respectively. In general, the two sequences to be compared are aligned to give a maximum correlation between the sequences. The alignment of the two sequences is examined and the number of positions giving an exact amino acid or nucleotide correspondence between the two sequences determined, divided by the total length of the alignment and multiplied by 100 to give a % identity figure. This % identity figure may be determined over the whole length of the sequences to be compared, which is particularly suitable for sequences of the same or very similar length and which are highly homologous, or over shorter defined lengths, which is more suitable for sequences of unequal length or which have a lower level of homology. There are a number of algorithms, and computer programs based thereon, which are available to be used the literature and/or publicly or commercially available for performing alignments and percent identity. The selection of the algorithm or program is not a limitation.

Examples of suitable alignment programs including, e.g., the software CLUSTALW under Unix and then be imported into the Bioedit program (Hall, T. A. 1999, BioEdit: a user-friendly biological sequence alignment editor and analysis program for Windows 95/98/NT. Nucl. Acids. Symp. Ser. 41:95-98); the Clustal Omega available from EMBL-EBI (Sievers, Fabian, et al. “Fast, scalable generation of high-quality protein multiple sequence alignments using Clustal Omega.” Molecular systems biology 7.1 (2011): 539 and Goujon, Mickael, et al. “A new bioinformatics analysis tools framework at EMBL-EBI.” Nucleic acids research 38.suppl 2 (2010): W695-W699); the Wisconsin Sequence Analysis Package, version 9.1 (Devereux J. et al., Nucleic Acids Res., 12:387-395, 1984, available from Genetics Computer Group, Madison, Wis., USA). The programs BESTFIT and GAP, may be used to determine the % identity between two polynucleotides and the % identity between two polypeptide sequences.

Other programs for determining identity and/or similarity between sequences include, e.g, the BLAST family of programs available from the National Center for Biotechnology Information (NCB), Bethesda, Md., USA and accessible through the home page of the NCBI at www.ncbi.nlm.nih.gov), the ALIGN program (version 2.0) which is part of the GCG sequence alignment software package. When utilizing the ALIGN program for comparing amino acid sequences, a PAM 120 weight residue table, a gap length penalty of 12, and a gap penalty of 4 can be used; and FASTA (Pearson W. R. and Lipman D. J., Proc. Natl. Acad. Sci. USA, 85:2444-8, 1988, available as part of the Wisconsin Sequence Analysis Package). SeqWeb Software (a web-based interface to the GCG Wisconsin Package: Gap program).

In one embodiment, the expression cassette is designed for expression in the heart, including the cardiac microtubules. The regulatory control elements typically contain a promoter sequence as part of the expression control sequences, e.g., located between the selected 5′ ITR sequence and the coding sequence. Constitutive promoters, regulatable promoters [see. e.g., WO 2011/126808 and WO 2013/04943], tissue specific promoters, or a promoter responsive to physiologic cues may be used may be utilized in the vectors described herein.

Examples of constitutive promoters suitable for controlling expression of the therapeutic products include, but are not limited to chicken β-actin (CB) promoter, human cytomegalovirus (CMV) promoter, ubiquitin C promoter (UbC), the early and late promoters of simian virus 40 (SV40), U6 promoter, metallothionein promoters, EF1α promoter, ubiquitin promoter, hypoxanthine phosphoribosyl transferase (HPRT) promoter, dihydrofolate reductase (DHFR) promoter (Scharfmann et al., Proc. Natl. Acad. Sci. USA 88:4626-4630 (1991), adenosine deaminase promoter, phosphoglycerol kinase (PGK) promoter, pyruvate kinase promoter phosphoglycerol mutase promoter, the D-actin promoter (Lai et al., Proc. Natl. Acad. Sci. USA 86: 10006-10 (1989), the long terminal repeats (LTR) of Moloney Leukemia Virus and other retroviruses, the thymidine kinase promoter of Herpes Simplex Virus and other constitutive promoters known to those of skill in the art. Examples of tissue- or cell-specific promoters suitable for use in certain embodiments include, but are not limited to, endothelin-I (ET-I) and Flt-I, which are specific for endothelial cells, FoxJ1 (that targets ciliated cells).

Inducible promoters suitable for controlling expression of the therapeutic product include promoters responsive to exogenous agents (e.g., pharmacological agents) or to physiological cues. These response elements include, but are not limited to a hypoxia response element (HRE) that binds HIF-Iα and β, a metal-ion response element such as described by Mayo et al. (1982, Cell 29:99-108); Brinster et al. (1982, Nature 296:39-42) and Searle et al. (1985, Mol. Cell. Biol. 5:1480-1489); or a heat shock response element such as described by Nouer et al. (in: Heat Shock Response, ed. Nouer, L., CRC, Boca Raton, Fla., ppI67-220, 1991).

In one embodiment, expression of the neutralizing antibody construct is controlled by a regulatable promoter that provides tight control over the transcription of the gene encoding the neutralizing antibody construct, e.g., a pharmacological agent, or transcription factors activated by a pharmacological agent or in alternative embodiments, physiological cues. Promoter systems that are non-leaky and that can be tightly controlled are preferred. Examples of regulatable promoters which are ligand-dependent transcription factor complexes that may be used in certain embodiments include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In certain embodiments, the gene switch is an EcR-based gene switch. Examples of such systems include, without limitation, the systems described in U.S. Pat. Nos. 6,258,603, 7,045,315, U.S. Published Patent Application Nos. 2006/0014711, 2007/0161086, and International Published Application No. WO 01/70816. Examples of chimeric ecdysone receptor systems are described in U.S. Pat. No. 7,091,038, U.S. Published Patent Application Nos. 2002/0110861, 2004/0033600, 2004/0096942, 2005/0266457, and 2006/0100416, and International Published Application Nos. WO 01/70816, WO 02/066612, WO 02/066613, WO 02/066614, WO 02/066615, WO 02/29075, and WO 2005/108617, each of which is incorporated by reference in its entirety. An example of a non-steroidal ecdysone agonist-regulated system is the RheoSwitch® Mammalian Inducible Expression System (New England Biolabs, Ipswich, Mass.).

Still other promoter systems may include response elements including but not limited to a tetracycline (tet) response element (such as described by Gossen & Bujard (1992, Proc. Natl. Acad. Sci. USA 89:5547-551); or a hormone response element such as described by Lee et al. (1981, Nature 294:228-232); Hynes et al. (1981, Proc. Natl. Acad. Sci. USA 78:2038-2042); Klock et al. (1987, Nature 329:734-736); and Israel & Kaufman (1989, Nucl. Acids Res. 17:2589-2604) and other inducible promoters known in the art. Using such promoters, expression of the neutralizing antibody construct can be controlled, for example, by the Tet-on/off system (Gossen et al., 1995, Science 268:1766-9; Gossen et al., 1992, Proc. Natl. Acad. Sci. USA 89(12):5547-51); the TetR-KRAB system (Urrutia R., 2003, Genome Biol., 4(10):231; Deuschle U et al., 1995, Mol Cell Biol. (4):1907-14); the mifepristone (RU486) regulatable system (Geneswitch; Wang Y et al., 1994, Proc. Natl. Acad. Sci. USA 91(17):8180-4; Schillinger et al., 2005, Proc. Natl. Acad. Sci. USA 102(39):13789-94); the humanized tamoxifen-dep regulatable system (Roscilli et al., 2002, Mol. Ther. 6(5):653-63). The gene switch may be based on heterodimerization of FK506 binding protein (FKBP) with FKBP rapamycin associated protein (FRAP) and is regulated through rapamycin or its non-immunosuppressive analogs. Examples of such systems, include, without limitation, the ARGENT™ Transcriptional Technology (ARIAD Pharmaceuticals, Cambridge, Mass.) and the systems described in U.S. Pat. Nos. 6,015,709, 6,117,680, 6,479,653, 6,187,757, and 6,649,595, U.S. Publication No. 2002/0173474, U.S. Publication No. 200910100535, U.S. Pat. No. 5,834,266, U.S. Pat. No. 7,109,317, U.S. Pat. No. 7,485,441, U.S. Pat. No. 5,830,462, U.S. Pat. No. 5,869,337, U.S. Pat. No. 5,871,753, U.S. Pat. No. 6,011,018, U.S. Pat. No. 6,043,082, U.S. Pat. No. 6,046,047, U.S. Pat. No. 6,063,625, U.S. Pat. No. 6,140,120, U.S. Pat. No. 6,165,787, U.S. Pat. No. 6,972,193, U.S. Pat. No. 6,326,166, U.S. Pat. No. 7,008,780, U.S. Pat. No. 6,133,456, U.S. Pat. No. 6,150,527, U.S. Pat. No. 6,506,379, U.S. Pat. No. 6,258,823, U.S. Pat. No. 6,693,189, U.S. Pat. No. 6,127,521, U.S. Pat. No. 6,150,137, U.S. Pat. No. 6,464,974, U.S. Pat. No. 6,509,152, U.S. Pat. No. 6,015,709, U.S. Pat. No. 6,117,680, U.S. Pat. No. 6,479,653, U.S. Pat. No. 6,187,757, U.S. Pat. No. 6,649,595, U.S. Pat. No. 6,984,635, U.S. Pat. No. 7,067,526, U.S. Pat. No. 7,196,192, U.S. Pat. No. 6,476,200, U.S. Pat. No. 6,492,106, WO 94/18347, WO 96/20951, WO 96/06097, WO 97/31898, WO 96/41865, WO 98/02441, WO 95/33052, WO 99110508, WO 99110510, WO 99/36553, WO 99/41258, WO 01114387, ARGENT™ Regulated Transcription Retrovirus Kit, Version 2.0 (9109102), and ARGENT™ Regulated Transcription Plasmid Kit, Version 2.0 (9109/02), each of which is incorporated herein by reference in its entirety. The Ariad system is designed to be induced by rapamycin and analogs thereof referred to as “rapalogs”. Examples of suitable rapamycins are provided in the documents listed above in connection with the description of the ARGENT™ system. In one embodiment, the molecule is rapamycin [e.g., marketed as Rapamune™ by Pfizer]. In another embodiment, a rapalog known as AP21967 [ARIAD] is used. Examples of these dimerizer molecules that can be used include, but are not limited to rapamycin, FK506, FK1012 (a homodimer of FK506), rapamycin analogs (“rapalogs”) which are readily prepared by chemical modifications of the natural product to add a “bump” that reduces or eliminates affinity for endogenous FKBP and/or FRAP. Examples of rapalogs include, but are not limited to such as AP26113 (Ariad), AP1510 (Amara, J. F., et al., 1997, Proc Natl Acad Sci USA, 94(20): 10618-23) AP22660, AP22594, AP21370, AP22594, AP23054, AP1855, AP1856, AP1701, AP1861, AP1692 and AP1889, with designed ‘bumps’ that minimize interactions with endogenous FKBP. Still other rapalogs may be selected, e.g., AP23573 [Merck].

Other suitable enhancers include those that are appropriate for a desired target tissue indications. In one embodiment, the expression cassette comprises one or more expression enhancers. In one embodiment, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g, the chicken beta-actin intron. Other suitable introns include those known in the art, e.g., such as are described in WO 2011/126808. Examples of suitable polyA sequences include, e.g., rabbit binding globulin (rBG), SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence [see, e.g., M A Zanta-Boussif, et al, Gene Therapy (2009) 16: 605-619].

In certain embodiments, the TTL coding sequences are engineered in a non-viral vector. Such a non-viral vector may be a plasmid carrying an expression cassette which includes, at a minimum, the TTL coding sequence and optionally, a promoter (e.g. a cardiac troponin T (cTNT) promoter sequence) or other regulatory elements, which is delivered to the heart. Non-viral delivery of nucleic acid molecules to smooth and cardiac muscle systems may include chemical or physical methods. Chemical methods include the use of cationic liposomes (“lipoplex”), polymers (“polyplex”), combinations of the two (“lipopolyplex”), calcium phosphate, and DEAE dextran. Additionally, or optionally, such nucleic acid molecules may be used in a composition further comprising one or more reagents, including, e.g., liposomal reagents such as, e.g., DOTAP/DOPE, Lipofectin, Lipofectamine, etc, and cationic polymers such as PEI, Effectene, and dendrimers. Such reagents are effective for transfecting smooth muscle cells. In addition to the chemical methods, a number of physical methods exist that promote the direct entry of uncomplexed DNA into the cell. These methods can include microinjection of individual cells, hydroporation, electroporation, ultrasound, and biolistic delivery (i.e., the gene gun).

In certain embodiments, an expression cassette comprising the TTL gene is carried by a viral vector, e.g., a recombinant adenovirus, lentivirus, or adeno-associated virus. In such embodiments, the viral vector may be a replication-defective virus.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

### Replication-Defective Adenovirus Vectors

In one embodiment, replication-defective adenoviral vectors are used. Any of a number of suitable adenoviruses may be used as a source of the adenoviral capsid sequence and/or in production. See, e.g., U.S. Pat. Nos. 9,617,561; 9,592,284; 9,133,483; 8,846,031; 8,603,459; 8,394,386; 8,105,574; 7,838,277; 7,344,872; 8,387,368; 6,365,394; 6,287,571; 6,281,010; 6,270,996; 6,261,551; 6,251,677; 6,203,975; 6,083,716; 6,019,978; 6,001,557; 5,872,154; 5,871,982; 5,856,152; 5,698,202. Still other adenoviruses are available from the American Type Culture Collection. In one embodiment, the adenoviral particles are rendered replication-defective by deletions in the E1a and/or E1b genes. Alternatively, the adenoviruses are rendered replication-defective by another means, optionally while retaining the E1a and/or E1b genes. The adenoviral vectors can also contain other mutations to the adenoviral genome, e.g., temperature-sensitive mutations or deletions in other genes. In other embodiments, it is desirable to retain an intact E1a and/or E1b region in the adenoviral vectors. Such an intact E1 region may be located in its native location in the adenoviral genome or placed in the site of a deletion in the native adenoviral genome (e.g., in the E3 region).

In the construction of useful adenovirus vectors for delivery of a gene to the human (or other mammalian) cell, a range of adenovirus nucleic acid sequences can be employed in the vectors. For example, all or a portion of the adenovirus delayed early gene E3 may be eliminated from the adenovirus sequence which forms a part of the recombinant virus. The function of E3 is believed to be irrelevant to the function and production of the recombinant virus particle. Adenovirus vectors may also be constructed having a deletion of at least the ORF6 region of the E4 gene, and more desirably because of the redundancy in the function of this region, the entire E4 region. Still another adenoviral vector contains a deletion in the delayed early gene E2a. Deletions may also be made in any of the late genes L1 through L5 of the adenovirus genome. Similarly, deletions in the intermediate genes IX and IVa₂ may be useful for some purposes. Other deletions may be made in the other structural or non-structural adenovirus genes. The above discussed deletions may be used individually, i.e., an adenovirus sequence for use as described herein may contain deletions in only a single region. Alternatively, deletions of entire genes or portions thereof effective to destroy their biological activity may be used in any combination. For example, in one exemplary vector, the adenovirus sequence may have deletions of the E1 genes and the E4 gene, or of the E1, E2a and E3 genes, or of the E1 and E3 genes, or of E1, E2a and E4 genes, with or without deletion of E3, and so on. As discussed above, such deletions may be used in combination with other mutations, such as temperature-sensitive mutations, to achieve a desired result.

An adenoviral vector lacking any essential adenoviral sequences (e.g., E1a, E1b, E2a, E2b, E4 ORF6, L1, L2, L3, L4 and L5) may be cultured in the presence of the missing adenoviral gene products which are required for viral infectivity and propagation of an adenoviral particle. These helper functions may be provided by culturing the adenoviral vector in the presence of one or more helper constructs (e.g., a plasmid or virus) or a packaging host cell. See, for example, the techniques described for preparation of a “minimal” human Ad vector in International Patent Application WO96/13597, published May 9, 1996, and incorporated herein by reference.

1. Helper Viruses

Thus, depending upon the adenovirus gene content of the viral vectors employed to carry the expression cassette, a helper adenovirus or non-replicating virus fragment may be necessary to provide sufficient adenovirus gene sequences necessary to produce an infective recombinant viral particle containing the expression cassette. Useful helper viruses contain selected adenovirus gene sequences not present in the adenovirus vector construct and/or not expressed by the packaging cell line in which the vector is transfected. In one embodiment, the helper virus is replication-defective and contains a variety of adenovirus genes in addition to the sequences described above. Such a helper virus is desirably used in combination with an E1-expressing cell line.

Helper viruses may also be formed into poly-cation conjugates as described in Wu et al, J. Biol. Chem., 264:16985-16987 (1989); K. J. Fisher and J. M. Wilson, Biochem. J., 299:49 (Apr. 1, 1994). Helper virus may optionally contain a second reporter minigene. A number of such reporter genes are known to the art. The presence of a reporter gene on the helper virus which is different from the transgene on the adenovirus vector allows both the Ad vector and the helper virus to be independently monitored. This second reporter is used to enable separation between the resulting recombinant virus and the helper virus upon purification.

2. Complementation Cell Lines

To generate recombinant adenoviruses (Ad) deleted in any of the genes described above, the function of the deleted gene region, if essential to the replication and infectivity of the virus, must be supplied to the recombinant virus by a helper virus or cell line, i.e., a complementation or packaging cell line. In many circumstances, a cell line expressing the human E1 can be used to transcomplement the Ad vector. However, in certain circumstances, it will be desirable to utilize a cell line which expresses the E1 gene products can be utilized for production of an E1-deleted adenovirus. Such cell lines have been described. See, e.g., U.S. Pat. No. 6,083,716.

If desired, one may utilize the sequences provided herein to generate a packaging cell or cell line that expresses, at a minimum, the adenovirus E1 gene under the transcriptional control of a promoter for expression in a selected parent cell line. Inducible or constitutive promoters may be employed for this purpose. Examples of such promoters are described in detail elsewhere in this specification. A parent cell is selected for the generation of a novel cell line expressing any desired adenovirus gene. Without limitation, such a parent cell line may be HeLa [ATCC Accession No. CCL 2], A549 [ATCC Accession No. CCL 185], HEK 293, KB [CCL 17], Detroit [e.g., Detroit 510, CCL 72] and WI-38 [CCL 75] cells, among others. These cell lines are all available from the American Type Culture Collection, 10801 University Boulevard, Manassas, Va. 20110-2209. Other suitable parent cell lines may be obtained from other sources.

Such E1-expressing cell lines are useful in the generation of recombinant adenovirus E1 deleted vectors. Additionally, or alternatively, cell lines that express one or more adenoviral gene products, e.g., E1a, E1b, E2a, and/or E4 ORF6, can be constructed using essentially the same procedures are used in the generation of recombinant viral vectors. Such cell lines can be utilized to transcomplement adenovirus vectors deleted in the essential genes that encode those products, or to provide helper functions necessary for packaging of a helper-dependent virus (e.g., adeno-associated virus). The preparation of a host cell involves techniques such as assembly of selected DNA sequences. This assembly may be accomplished utilizing conventional techniques. Such techniques include cDNA and genomic cloning, which are well known and are described in Sambrook et al., cited above, use of overlapping oligonucleotide sequences of the adenovirus genomes, combined with polymerase chain reaction, synthetic methods, and any other suitable methods which provide the desired nucleotide sequence.

In still another alternative, the essential adenoviral gene products are provided in trans by the adenoviral vector and/or helper virus. In such an instance, a suitable host cell can be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T1/2, HEK 293 cells or PERC6 (both of which express functional adenoviral E1) [Fallaux, F J et al, (1998), Hum Gene Ther, 9:1909-17], Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation; nor is the type of mammalian cell, i.e., fibroblast, hepatocyte, tumor cell, etc.

3. Assembly of Viral Particle and Transfection of a Cell Line

Generally, when delivering the vector comprising the minigene by transfection, the vector is delivered in an amount from about 5 μg to about 100 μg DNA, and preferably about 10 to about 50 μg DNA to about 1×10′ cells to about 1×10¹³ cells, and preferably about 10⁵ cells. However, the relative amounts of vector DNA to host cells may be adjusted, taking into consideration such factors as the selected vector, the delivery method and the host cells selected.

The vector may be any vector known in the art or disclosed above, including naked DNA, a plasmid, phage, transposon, cosmids, episomes, viruses, etc. Introduction into the host cell of the vector may be achieved by any means known in the art or as disclosed above, including transfection, and infection. One or more of the adenoviral genes may be stably integrated into the genome of the host cell, stably expressed as episomes, or expressed transiently. The gene products may all be expressed transiently, on an episome or stably integrated, or some of the gene products may be expressed stably while others are expressed transiently. Furthermore, the promoters for each of the adenoviral genes may be selected independently from a constitutive promoter, an inducible promoter or a native adenoviral promoter. The promoters may be regulated by a specific physiological state of the organism or cell (i.e., by the differentiation state or in replicating or quiescent cells) or by exogenously-added factors, for example.

Introduction of the molecules (as plasmids or viruses) into the host cell may also be accomplished using techniques known to the skilled artisan and as discussed throughout the specification. In preferred embodiment, standard transfection techniques are used, e.g., CaPO₄ transfection or electroporation. Assembly of the selected DNA sequences of the adenovirus (as well as the transgene and other vector elements into various intermediate plasmids, and the use of the plasmids and vectors to produce a recombinant viral particle are all achieved using conventional techniques. Such techniques include conventional cloning techniques of cDNA such as those described in texts [Sambrook et al, cited above], use of overlapping oligonucleotide sequences of the adenovirus genomes, polymerase chain reaction, and any suitable method which provides the desired nucleotide sequence. Standard transfection and co-transfection techniques are employed, e.g., CaPO₄ precipitation techniques. Other conventional methods employed include homologous recombination of the viral genomes, plaquing of viruses in agar overlay, methods of measuring signal generation, and the like.

Dosages of the viral vector will depend primarily on factors such as the condition being treated, the age, weight and health of the patient, and may thus vary among patients. For example, a therapeutically effective adult human or veterinary dosage of the viral vector is generally in the range of from about 100 μL to about 100 mL of a carrier containing concentrations of from about 1×10⁶ to about 1×10¹⁵ particles, about 1×10¹¹ to 1×10¹³ particles, or about 1×10⁹ to 1×10¹² particles virus. Dosages will range depending upon the size of the animal and the route of administration. For example, a suitable human or veterinary dosage (for about an 80 kg animal) for intramuscular injection is in the range of about 1×10⁹ to about 5×10¹² particles per mL, for a single site. Optionally, multiple sites of administration may be delivered. In another example, a suitable human or veterinary dosage may be in the range of about 1×10¹¹ to about 1×10¹⁵ particles for an oral formulation. One of skill in the art may adjust these doses, depending the route of administration, and the therapeutic or vaccinal application for which the recombinant vector is employed. The levels of expression of the transgene, or for an immunogen, the level of circulating antibody, can be monitored to determine the frequency of dosage administration. Yet other methods for determining the timing of frequency of administration will be readily apparent to one of skill in the art.

### Lentivirus Systems

A variety of different lentivirus systems are known in the art. See, e.g., WO2001089580 A1 for a method for obtaining stable cardiovascular transduction with a lentivirus system. See, e.g., U.S. Pat. No. 6,521,457. See, also, discussion in N B Wasala, et al, “The evolution of heart gene delivery vectors”, J Gen Med., 2011 October; 13(10): 557-565, which is incorporated herein by reference.

### Recombinant AAV

In some embodiments, ttl is expressed from a recombinant adeno-associated virus, and the vector genome also contains AAV inverted terminal repeats (ITRs). In one embodiment, the rAAV is pseudotyped, i.e., the AAV capsid is from a different source AAV than that the AAV which provides the ITRs. In one embodiment, the ITRs of AAV serotype 2 are used. However, ITRs from other suitable sources may be selected. Optionally, the AAV may be a self-complementary AAV.

Where the gene is to be expressed from an AAV, the expression cassettes described herein include an AAV 5′ inverted terminal repeat (ITR) and an AAV 3′ ITR. However, other configurations of these elements may be suitable. A shortened version of the 5′ ITR, termed ΔITR, has been described in which the D-sequence and terminal resolution site (trs) are deleted. In other embodiments, the full-length AAV 5′ and/or 3′ ITRs are used. Where a pseudotyped AAV is to be produced, the ITRs in the expression are selected from a source which differs from the AAV source of the capsid. For example, AAV2 ITRs may be selected for use with an AAV capsid having a particular efficiency for targeting CNS or tissues or cells within the CNS. In one embodiment, the ITR sequences from AAV2, or the deleted version thereof (ΔITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. However, other sources of AAV ITRs may be utilized.

As used herein, “recombinant AAV9 viral particle” refers to nuclease-resistant particle (NRP) which has an AAV9 capsid, the capsid having packaged therein a heterologous nucleic acid molecule comprising an expression cassette for a desired gene product. Such an expression cassette typically contains an AAV 5′ and/or 3′ inverted terminal repeat sequence flanking a gene sequence, in which the gene sequence is operably linked to expression control sequences. These and other suitable elements of the expression cassette are described in more detail below and may alternatively be referred to herein as the transgene genomic sequences. This may also be referred to as a “full” AAV capsid. Such a rAAV viral particle is termed “pharmacologically active” when it delivers the transgene to a host cell which is capable of expressing the desired gene product carried by the expression cassette.

In many instances, rAAV particles are referred to as “DNase resistant” However, in addition to this endonuclease (DNase), other endo- and exo-nucleases may also be used in the purification steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled around the expression cassette which is designed to deliver a transgene to a host cell and protects these packaged genomic sequences from degradation (digestion) during nuclease incubation steps designed to remove contaminating nucleic acids which may be present from the production process.

As used herein, “AAV9 capsid” refers to the AAV9 produced using the nucleic acid sequence of GenBank accession: AY530579, or a sequence having at least 70% identity thereto which encodes the amino acid sequence of GenBank accession: AAS99264, is incorporated by reference herein and the AAV vp1 capsid protein is reproduced in SEQ ID NO: 6. Some variation from this encoded sequence is encompassed by certain embodiments, which may include sequences having about 99% identity to the referenced amino acid sequence in GenBank accession:AAS99264, SEQ ID NO: 6 and U.S. Pat. No. 7,906,111 (also WO 2005/033321) (i.e., less than about 1% variation from the referenced sequence). Such AAV may include, e.g., natural isolates (e.g., hu31 or hu32), or variants of AAV9 having amino acid substitutions, deletions or additions, e.g., including but not limited to amino acid substitutions selected from alternate residues “recruited” from the corresponding position in any other AAV capsid aligned with the AAV9 capsid; e.g., such as described in U.S. Pat. No. 9,102,949, U.S. Pat. No. 8,927,514, US2015/349911; and WO 2016/049230A1. However, in other embodiments, other variants of AAV9, or AAV9 capsids having at least about 95% identity to the above-referenced sequences may be selected. See. e.g., US Published Patent Application No. 2015/0079038. Methods of generating the capsid, coding sequences therefore, and methods for production of rAAV viral vectors have been described. See. e.g., Gao et al, Proc. Natl. Acad. Sci. U.S.A. 100 (10), 6081-6 (2003) and US 2013/0045186A1.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See. e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

Methods of preparing AAV-based vectors are known. See, e.g., US Published Patent Application No. 2007/0036760 (Feb. 15, 2007), which is incorporated by reference herein. The use of AAV capsids of AAV9 are particularly well suited for the compositions and methods described herein. The sequences of AAV9 and methods of generating vectors based on the AAV9 capsid are described in U.S. Pat. No. 7,906,111; US2015/0315612; WO 2012/112832; which are incorporated herein by reference. However, other AAV capsids may be selected or generated. For example, the sequences of AAV 1, AAV5, and AAV6 are known as are methods of generating vectors. See, e.g., U.S. Pat. No. 7,282,199 B2, U.S. Pat. No. 7,790,449, and U.S. Pat. No. 8,318,480, which are incorporated herein by reference. The sequences of a number of such AAV are provided in the above-cited U.S. Pat. No. 7,282,199 B2, U.S. Pat. No. 7,790,449, U.S. Pat. No. 8,318,480, and U.S. Pat. No. 7,906,111, and/or are available from GenBank. The sequences of any of the AAV capsids can be readily generated synthetically or using a variety of molecular biology and genetic engineering techniques. Suitable production techniques are well known to those of skill in the art. See, e.g., Sambrook et al, Molecular Cloning: A Laboratory Manual, Cold Spring Harbor Press (Cold Spring Harbor, N.Y.). Alternatively, oligonucleotides encoding peptides (e.g., CDRs) or the peptides themselves can generated synthetically, e.g., by the well-known solid phase peptide synthesis methods (Merrifield, (1962) J. Am. Chem. Soc., 85:2149; Stewart and Young, Solid Phase Peptide Synthesis (Freeman, San Francisco, 1969) pp. 27-62). These and other suitable production methods are within the knowledge of those of skill in the art and are not a limitation.

The recombinant adeno-associated virus (AAV) described herein may be generated using techniques which are known. See. e.g., WO 2003/042397; WO 2005/033321, WO 2006/110689; U.S. Pat. No. 7,588,772 B2. Such a method involves culturing a host cell which contains a nucleic acid sequence encoding an AAV capsid; a functional rep gene; an expression cassette composed of, at a minimum, AAV inverted terminal repeats (ITRs) and a transgene; and sufficient helper functions to permit packaging of the expression cassette into the AAV capsid protein.

To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. he number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad-spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

In brief, the method for separating rAAV9 particles having packaged genomic sequences from genome-deficient AAV9 intermediates involves subjecting a suspension comprising recombinant AAV9 viral particles and AAV 9 capsid intermediates to fast performance liquid chromatography, wherein the AAV9 viral particles and AAV9 intermediates are bound to a strong anion exchange resin equilibrated at a pH of 10.2, and subjected to a salt gradient while monitoring eluate for ultraviolet absorbance at about 260 and about 280. Although less optimal for rAAV9, the pH may be in the range of about 10.0 to 10.4. In this method, the AAV9 full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2/9 serotype. Under these ionic conditions, a significant percentage of residual cellular DNA and proteins flow through the column, while AAV particles are efficiently captured.

In certain embodiments, the composition is specifically targeted (e.g., via direct injection) to the heart. In certain embodiments, the composition or gene of interest is specifically expressed in the heart (e.g., cardiomyocytes).

Methods for preferentially targeting cardiac cells and/or for minimizing off-target non-cardiac gene transfer have been described.

In certain embodiments, a method such as that in U.S. Pat. No. 7,399,750, is used to increase the dwell time of the vector carrying the gene of interest in the heart by the induction of hypothermia, isolation of the heart from circulation, and near or complete cardiac arrest. Permeabilizing agents are an essential component of this method and are used during the administration of the virus to increase the uptake of the virus by the cardiac cells. This method is particularly well suited to viral vectors, where the gene expression may be is highly specific to cardiac muscle and, in particularly in the case of rAAV vectors, expression may be maintained long-term, with no signs of myocardiac inflammation. Still other systems and techniques may be used including, without limitation, e.g., a “bio-pacemaker”, such as that described in U.S. Pat. No. 8,642,747, US-2011-0112510.

The term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

The term “about” encompasses a variation within and including ±10%, unless otherwise specified.

Unless defined otherwise in this specification, technical and scientific terms used herein have the same meaning as commonly understood by one of ordinary skill in the art and by reference to published texts, which provide one skilled in the art with a general guide to many of the terms used in the present application.

### Example 1

While the idea that a proliferated (and perhaps modified) MT network may mechanically interfere with contraction is attractive, the “microtubule hypothesis” has remained controversial (see refs. 16 and 17). Two significant limitations have hindered our understanding: 1) a reliance on blunt pharmacological tools (colchicine/taxol) that have off-target consequences; 2) a lack of direct observation of MTs under the stress and strain of the contractile cycle. Here we have characterized MTs under contractile loads using a high-resolution imaging technique, and directly tested how MT detyrosination may regulate load-bearing and the mechanical properties of the myocyte.

### Materials and Methods

### Animals

Animal care and procedures were approved and performed in accordance with the standards set forth by the University of Pennsylvania Institutional Animal Care and Use Committee and the Guide for the Care and Use of Laboratory Animals published by the US National Institutes of Health. Desmin knockout mice and WT littermates were provided by Dr. Robert Bloch and have been previously characterized (36). Experiments were performed blinded to genotype.

### Cell Isolation and Culture

Primary adult ventricular cardiomyocytes were isolated from male SD rats 8-12 weeks of age as previously described (1). Briefly, the heart is removed from an anesthetized rat and retrograde-perfused with an enzymatic digestion solution. The digested heart is then minced and triturated with glass pipettes to free individual cardiomyocytes. The cardiomyocytes are filtered and centrifuged gently to remove debris, other cell types and enzyme, then gradually reintroduced to physiological solution.

For some experiments viable rat cardiomyocytes were additionally enriched by centrifugation in 20% Opti-Prep (Axis-Shield PoC AS, Osli, Norway) cushion. After reintroduction to physiological solution the cardiomyocyte suspension was loaded on the 20% Opti-Prep cushion in Rat CM medium and centrifuged for 10 min at 400×g. Cells were collected from the cell layer between medium and cushion, diluted in Rat CM medium up to total volume of 10 ml and spun again for 5 minutes at 300 rpm (Solvall ST16 centrifuge). After that cells were resuspended in an appropriate volume of Rat CM medium and plated in 12 well plates.

Following isolation, cardiomyocytes were plated so that neighboring cells were not in direct contact, preventing reformation of junctions. Cardiomyocytes used acutely were maintained in normal Tyrode's (NT) solution for up to 6 hours. Cardiomyocytes used more than 12 hours after isolation were maintained at 37° C. and 5% CO₂ in Rat CM medium. Cyto-D was added at 25 μM to Rat CM medium to prevent hypercontraction and myocyte death in the first 12 hours, but was washed out in the solution used for buckling experiments.

Viral constructs were permitted to express for 24-72 hours with Moi=100-200. Parthenolide (Fisher Scientific NC9013142) treatment was carried out at room temperature for 2 hours at 10 μM. All experiments were performed within 48 hours of isolation, except those involving shTTL, which were allowed 72 hours to improve knockdown efficiency. Of note, mouse myocytes do not respond well to long-term culture and viral transduction, and therefore these experiments were performed on freshly isolated cells.

Rat CM medium: 199 Medium (GIBCO, 11150-59) supplemented with 1× Insulin-Transferrin-Selenium-X solution, (GIBCO, 51500-056), 1/500 Primocin, (InvivoGen, ant-pm-1) and 20 mM HEPES at pH7.4.

Normal Tyrode's (NT) Solution: NaCl—140 mM; MgCl2—0.5 mM; NaH2PO4—0.33 mM; HEPES—5 mM; Glucose—5.5 mM; CaCl2—1.8 mM; KCl—5 mM; NaOH—pH to 7.4.

### Viral Constructs

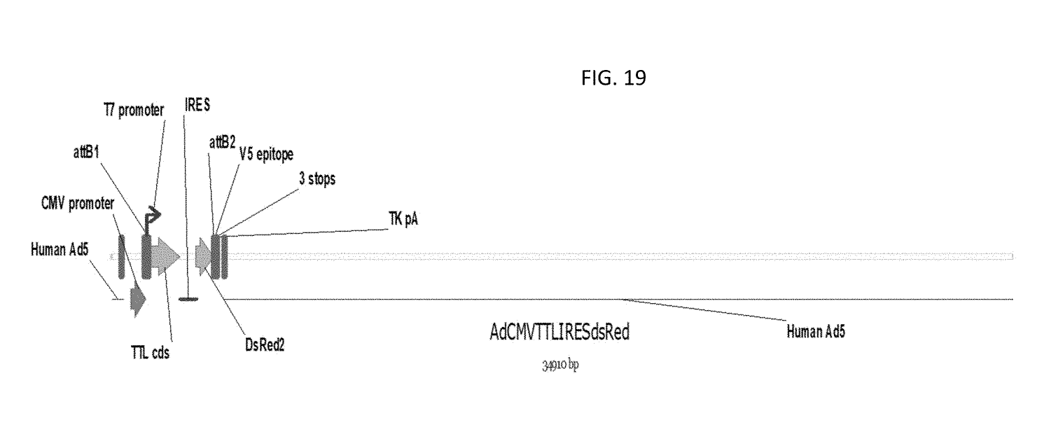

To create adenoviral vectors expressing human tubulin tyrosine ligase (TTL) transcriptionally fused with dsRed fluorescent protein and ensconsin microtubule-binding domain (EMTB) translationally fused with 3 copies of enhanced green fluorescent protein (EGFP), the corresponding cDNAs were inserted into pENTR for further Gateway recombination in adenoviral expression plasmids (See FIG. 19 and SEQ ID NOs: 1-5).

TTL-IRES-dsRed and EMTB-3xEGFP cDNA's were released from corresponding plasmids (courtesy of Dr. Christopher Ward and Dr. Erika Holzbaur, respectively) at BglII and NotI flanking sites and ligated to pENTR4 plasmid (Invitrogen) opened at EcoRV and Nod sites.

The pENTR4 shTTLRNA expression construct includes two expression cassettes. The first cassette consists of two shRNA coding TTL targeted oligonucleotides which are introduced downstream of RNA PolIII promoters, U6 and 7SK. To avoid transcription interference, promoters were directed head-to-head and mouse CMV enhancer was placed in between them. Second cassette contains an EF1α promoter that drives constitutively active mCherry fluorescent protein expression. The selection of a target sequences for TTL knockdown and shRNA design was done by BLOCK-iT RNAi Designer web resource (https://maidesigner.thermofisher.com/maiexpress/). The search was performed in conservative regions of TTL cDNA that share 100% identity among rat, human and mouse. Two sequences, GCTTCAGAACCATATCATGTT (SEQ ID NO: 7) and GTGCACGTGATCCAGAAAT (SEQ ID NO: 8) were chosen as a target sites.

All constructs were then transferred by Gateway recombinase into adenoviral expression plasmid pAdCMVN/V5/DEST (Invitrogen). Finally, recombinant adenoviral vectors were produced and amplified in HEK 293A cells according to manufacturer's protocol (ViraPower Adenoviral Expression System; Invitrogen). Viruses were isolated by CsCl gradient centrifugation and dialyzed against a 5% sucrose buffer (37). The titers of viral stocks measured by plaque assays were ˜5×10¹⁰ ffu/mL.

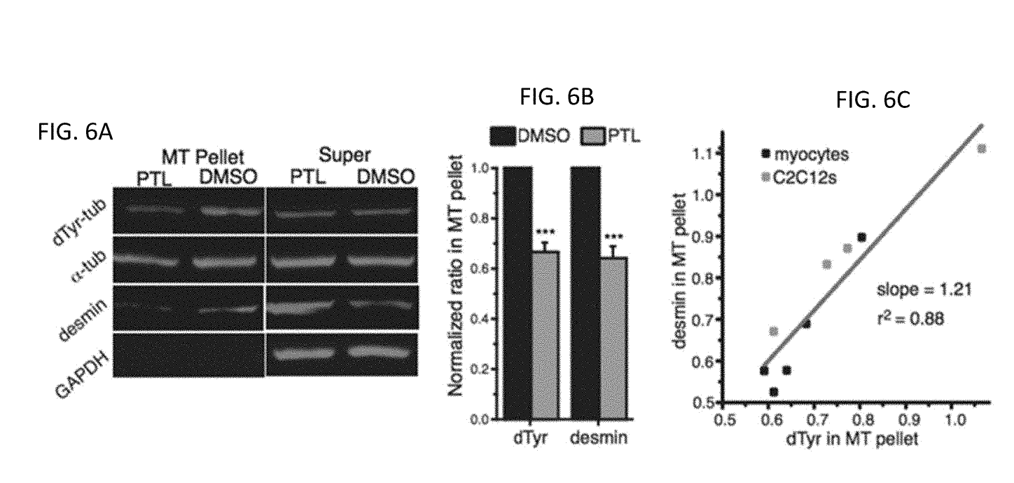

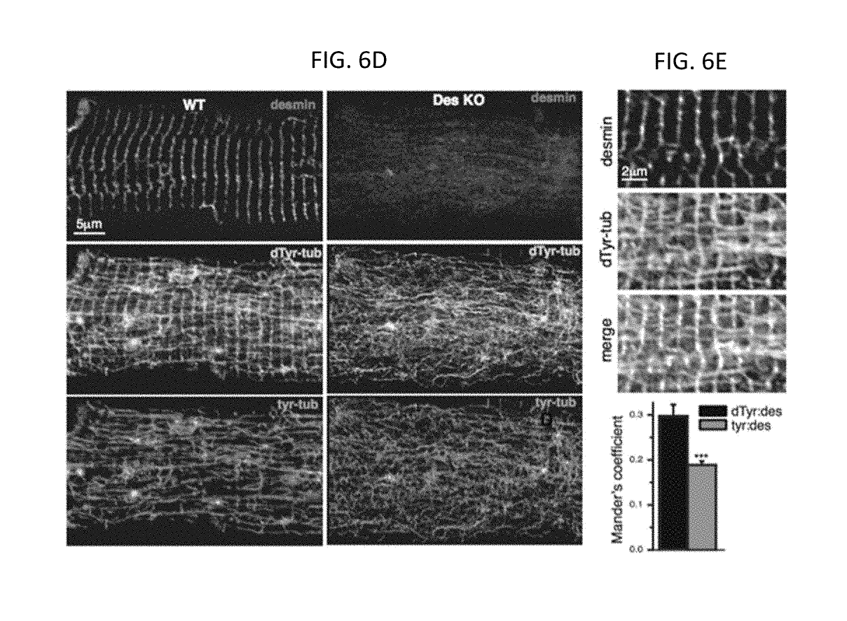

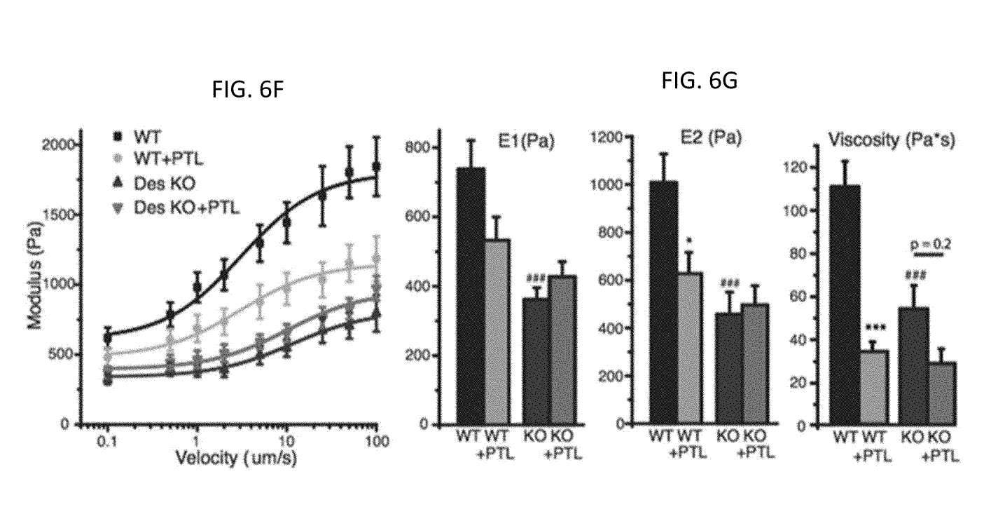

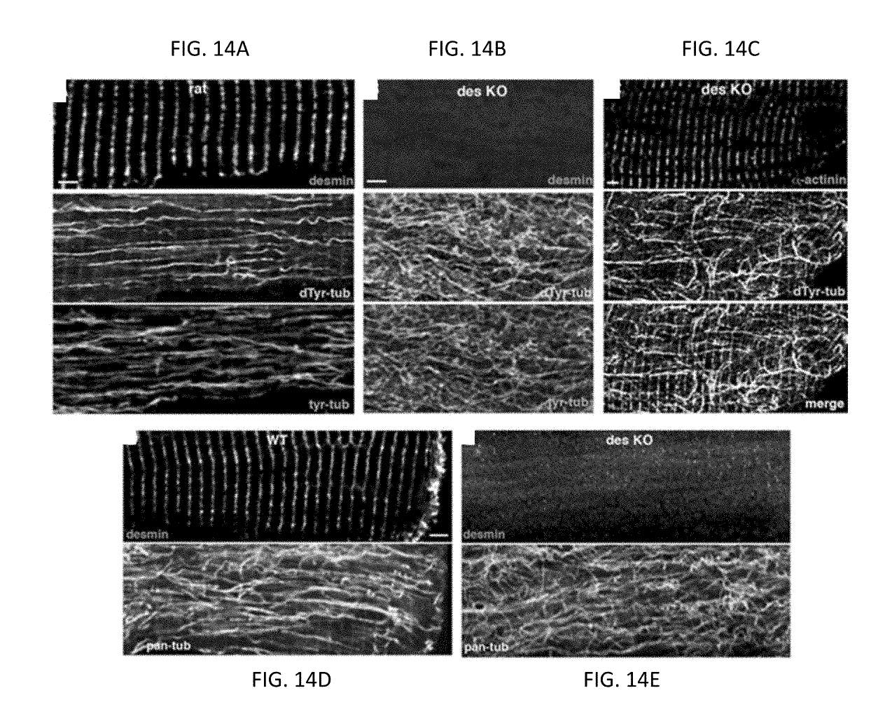

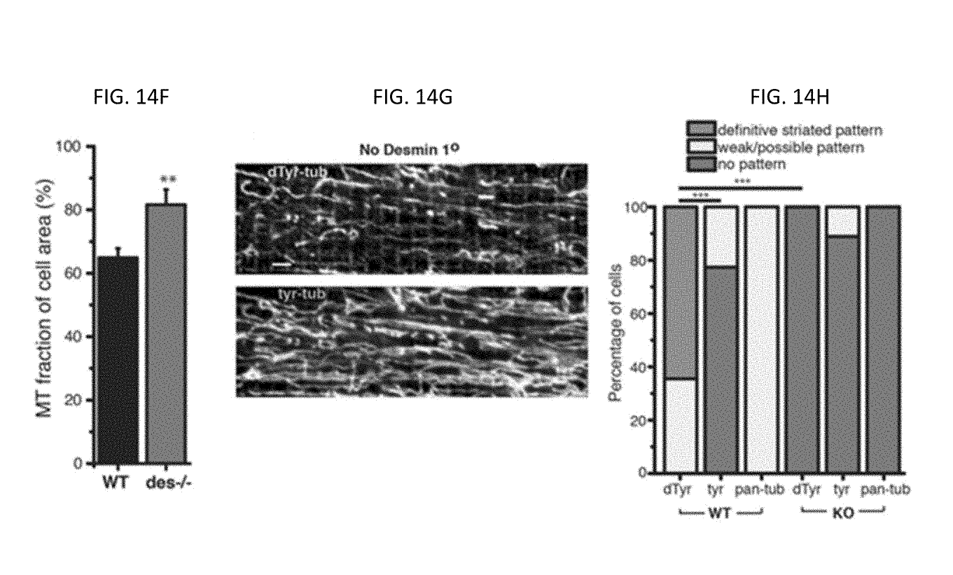

SiR Tubulin SiR Tubulin (Cytoskeleton, #CY-SC006) was loaded into rat myocytes at 125 nM final concentration at 37° C. in M199 media for 3 hrs. Of note, this concentration was insufficient for proper MT labeling in mouse myocytes. Higher concentrations produced better labeling, yet resulted in decreased contractility, precluding quantification of MT buckling in mouse myocytes. This poor labeling was particularly evident in desmin KO myocytes, where labeling is complicated by the denser and more disorganized MT cytoskeleton (FIG. 6D, FIG. 14A-FIG. 14E).

### Cell Contractility and Stretch

Experiments were performed in custom-fabricated cell chambers (Ionoptix) mounted on an LSM Zeiss 780 inverted confocal microscope using a 40× oil 1.4 NA objective and transmitted light camera (IonOptix MyoCam-S). For contractility assays, cells were maintained in NT solution and electrical field stimulation was provided at 1 Hz with a myopacer (IonOptix MYP100) through platinum electrodes lowered into the bath. Sarcomere length was measured optically by Fourier transform analysis (IonWizard, IonOptix). After 15 s of 1 Hz pacing to achieve steady state, five traces were recorded and analyzed.

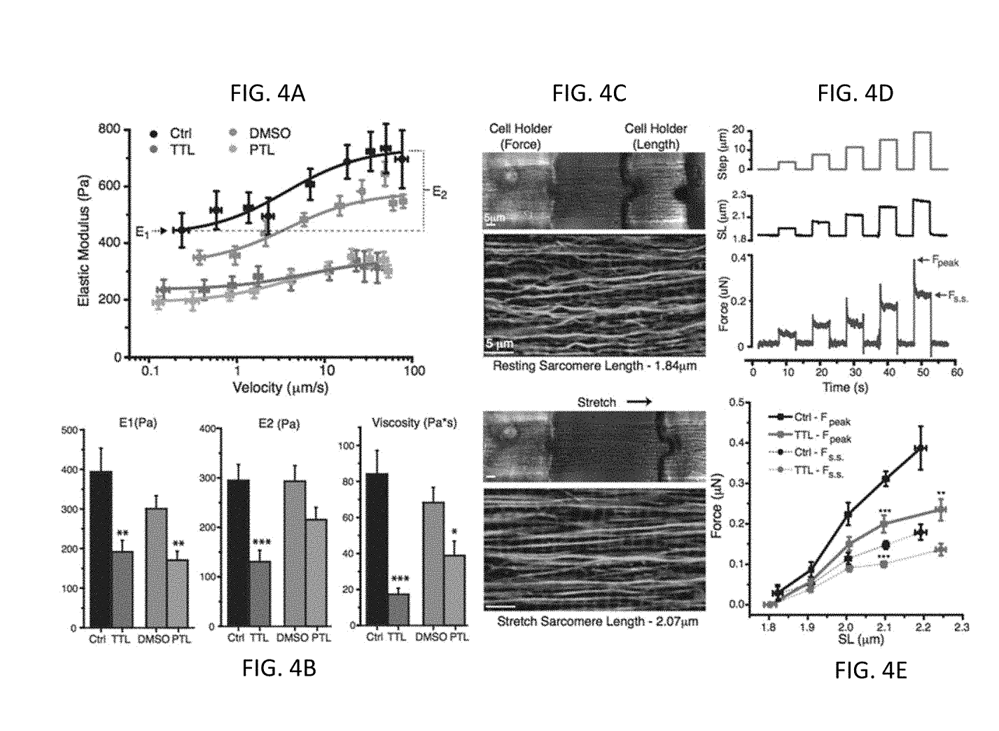

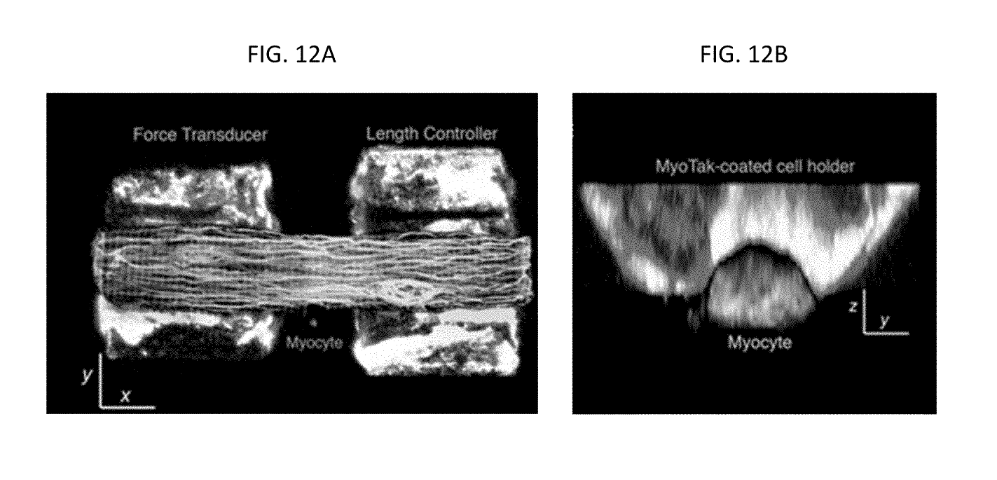

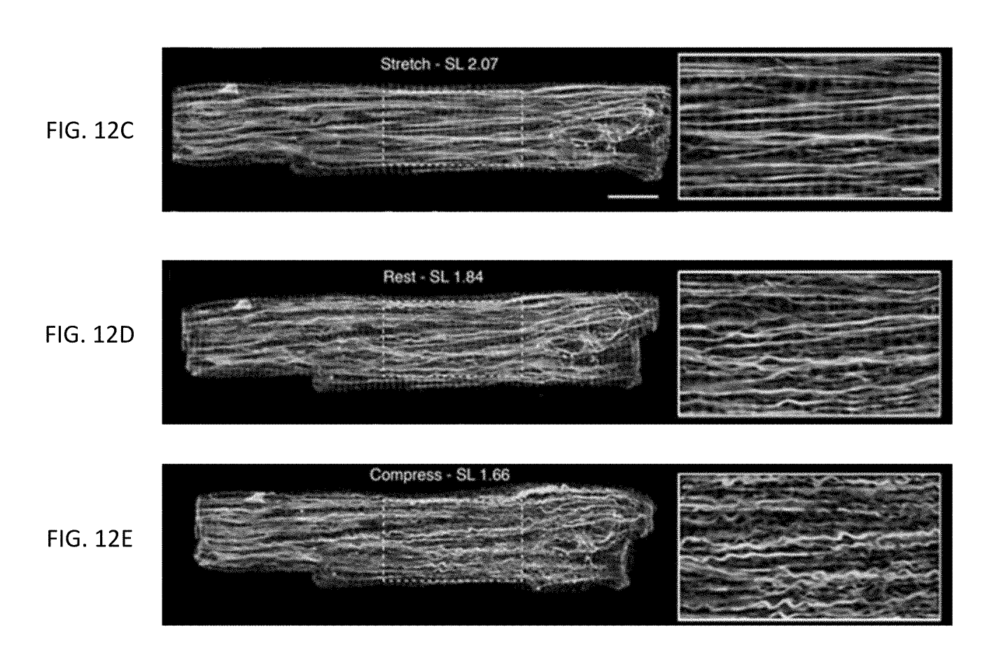

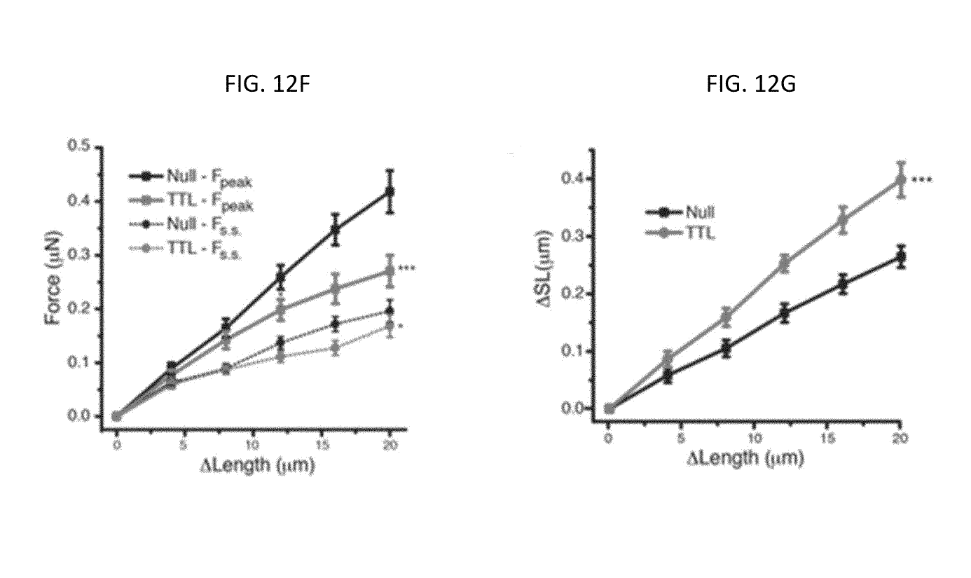

Cell stretch experiments were carried out as previously described (1) but with the following modifications. Cells were attached via MyoTak™ (Ionoptix) adhesive to glass cell holders with a laser-etched cavity custom fit for a cardiomyocyte (30 μm wide by 8 μm deep, FIG. 4C, FIG. 12A-FIG. 12E). One cell holder was mounted on a piezoelectric length controller and the other to a high-sensitivity optical force transducer (IonOptix OFT-100). 4 μm step-like increases in length were applied via the piezo by a 50 ms ramp and then held at a constant length for 5 s before releasing the stretch, while force and sarcomere length were continuously recorded at 1 KHz. While amplitude was increased with each step, the duration of the ramp to peak amplitude was held constant (50 ms), resulting in a progressively increasing velocity of stretch. Force recordings were filtered with a 100 Hz low-pass Bessel filter. Cells with a resting sarcomere length below 1.7 μm were considered hypercontracted and discarded from these studies. Force and sarcomere length traces were analyzed in IonWizard (IonOptix).

### Calcium Measurements

Myocytes were loaded with Fluo-3 by 20 min incubation with 1 μM Fluo-3-acetoxymethyl ester (Invitrogen) and 0.01% Pluronic F127 (a poloxamer made by BASF, Florham Park N.J., USA), and allowed an additional 10 min for de-esterification. Cells were scanned using a 488 nm argon ion laser in confocal line-scan mode at 1.92 ms/line. Cells were electrically paced at 1 Hz for 20 s to achieve steady state; the final 5 traces of the pacing protocol were pooled and analyzed for calcium transient properties. The measured fluorescence (F) throughout the transient was normalized to the resting fluorescence prior to stimulation (F₀) to normalize for heterogeneity in dye loading.

### Immunofluorescence

Cells were fixed in 4% PFA for 15 minutes and permeabilized in 0.5% TritonX-100 for 4 minutes at room temperature. Alternately cells were fixed in methanol at −20° C. for 7 minutes. After washing, cells were placed in blocking buffer (5% BSA and 0.1% TritonX-100 in PBS) for 1 hour, then labeled with primary antibodies (below) for 24-48 hours at 4° C. Cells were then washed 4× in blocking buffer, labeled with secondary antibodies (below) at room temperature and washed 3× in blocking buffer. Stained cells were mounted on mouse laminin coated #1.5 coverslips in Prolong Gold Antifade Mountant (Life Technologies P36934) for imaging.

### Western Blotting

For analysis of proteins expression levels quantitative Western blots were performed using infrared fluorescence imaging on an Odyssey Imager (LI-COR). Cell homogenates were prepared in ice-cold IGEPAL lysis buffer. After 1 freezing cycle lysates were spun at 18,000×g for 5 min. Aliquots of supernatants were mixed with 4× sample buffer (LI-COR, 928-40004) containing 10% BME, boiled for 6 min., and resolved on sodium dodecyl sulfate (SDS)-polyacrylamide gel electrophoresis Tris-glycine gels (Bio-Rad). Proteins were transferred to a membrane on Mini Trans-Blot Cell (Bio-Rad), blocked 1 h in Odyssey Blocking Buffer (TBS) (LI-COR, 927-50000), and probed with the corresponding primary antibody (see list below) for 2 h at room temperature. Membranes were then rinsed with TBST 4 times for 5 min, and incubated with secondary antibodies (below) for 1.5 h at room temperature. Membranes were rinsed again with TBST and then imaged on Odyssey Imager. Image analysis was performed using Image Studio Lite software (LI-COR). Fluorescent band intensity was always normalized to GAPDH loading control. Analysis of human tissue samples for levels of detyrosinated tubulin was performed blinded to the experimental groups.

IGEPAL lysis buffer: 50 mM Tris, pH 8.0 containing 1% IGEPAL CA-630 detergent (Sigma, 13021), 159 mM NaCl, PIC (Sigma, 1378), and PMSF (Sigma, 78830) 1 mM.

### Antibodies and Labels

Alpha tubulin; mouse monoclonal, clone DM1A (Cell Signaling #3873)-IF 1:200; WB 1:1000 Tyrosinated tubulin; mouse monoclonal, clone TUB-1A2 (Sigma T 9028); WB 1:1000 Detyrosinated tubulin; rabbit polyclonal (abcam ab48389); IF 1:200; WB 1:500 Tubulin tyrosine ligase; rabbit polyclonal (proteintech 13618-1-AP); WB 1:500 GAPDH; mouse monoclonal, clone 3B1E9 (GenScript A01622-40); WB 1:1000 Desmin; goat polyclonal (R&D AF3844); IF 1:200 (primary labeled Atto 565) Goat anti-mouse AF 647 (Life Technologies, A-21235); IF 1:500 Goat anti-rabbit AF 488 (Life Technologies, A-11034); IF 1:500 IRDye800CW (LI-COR, #925-32210); WB 1:10000 IRDye680RD (LI-COR, #925-68071); WB 1:10000 Lightning Link Rapid Atto565 (Innova Biosciences, 351-0030).

### Co-Sedimentation

Microtubules were isolated for co-sedimentation from isolated cardiomyocytes or C2C12 cells following treatment with parthenolide. Cells were homogenized at 37° C. in 400 μl of microtubule-stabilizing buffer (MSB) by passage through 25G syringe needle. Homogenate was centrifuged to remove cell debris at 2000×g for 5 min at 37° C. and pellet was resuspended in RIPA buffer containing 5 mM CaCl₂. Supematant containing free and polymerized tubulin was loaded on a cushion of 10% sucrose in MSB and centrifuged in SW-60 rotor at 100000×g for 30 min at 37° C. Supernatant was saved, pellet (polymerized MT fraction) was resuspended in 0° C. RIPA buffer containing 5 mM CaCl₂ and incubated on ice for 10 min. All samples were then centrifuged at 14000×g for 5 min at 0° C. and supernatant was measured by Bradford assay against a BSA (Sigma) standard and boiled in 12-40 μl of 1× loading buffer (Li-COR, 928-40004) prior to western blotting.

MSB: [100 mM PIPES pH6.9, 5 mM MgCl2, 1 mM EGTA, 30% (v/v) glycerol, 0.2% Nonidet P40, 0.2% TritonX-100, 0.2% Tween 20, 0.1% beta-mercaptoethanol, 0.001% Antifoam, DMSO 3% (final concentration), 1 mM ATP, 100 μM GTP, 2× Protease Inhibitor Cocktail (Sigma P8340), and 1 mM PMSF

### Imaging Equipment and Analysis

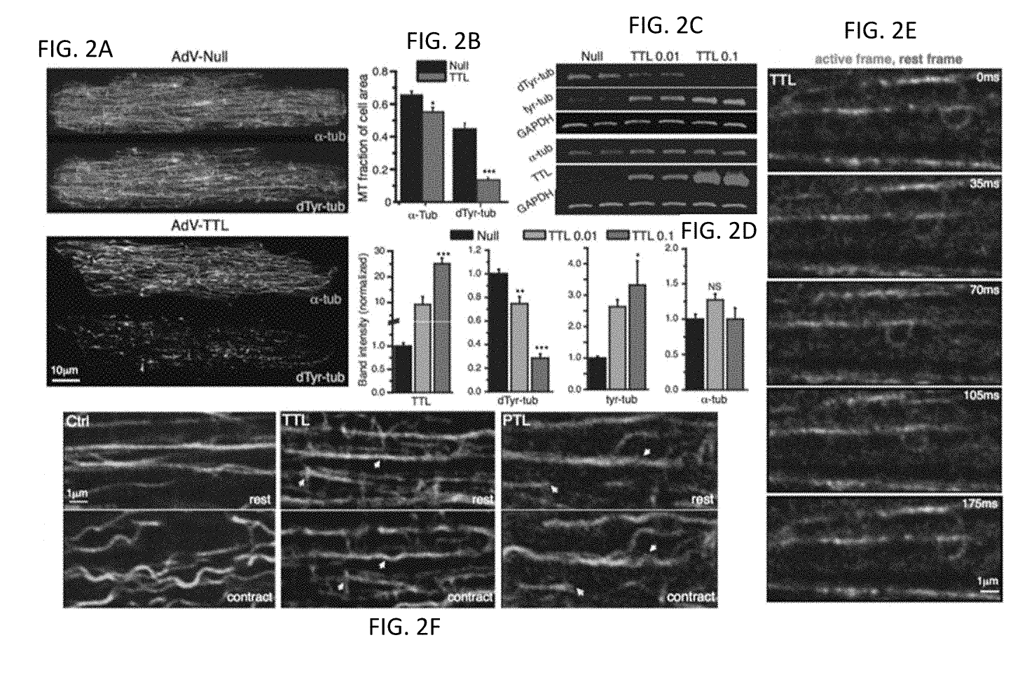

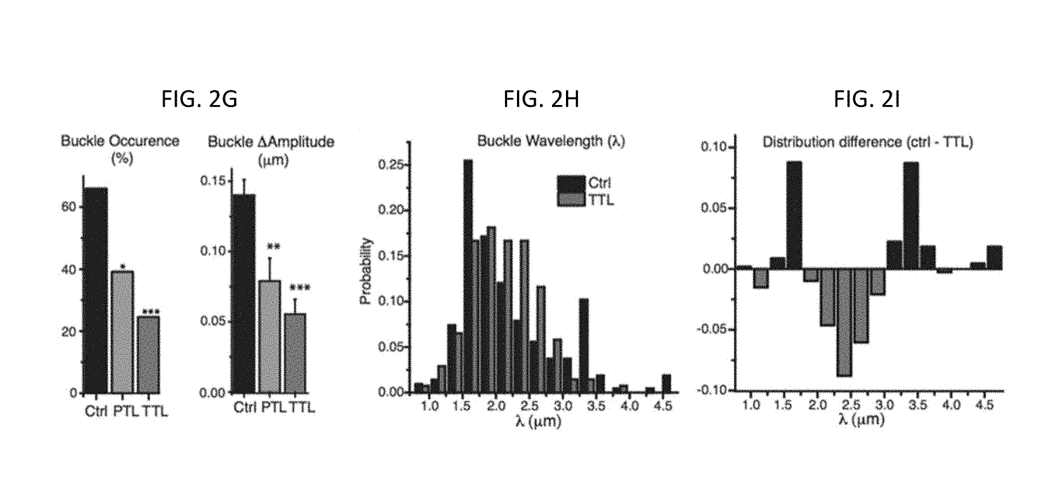

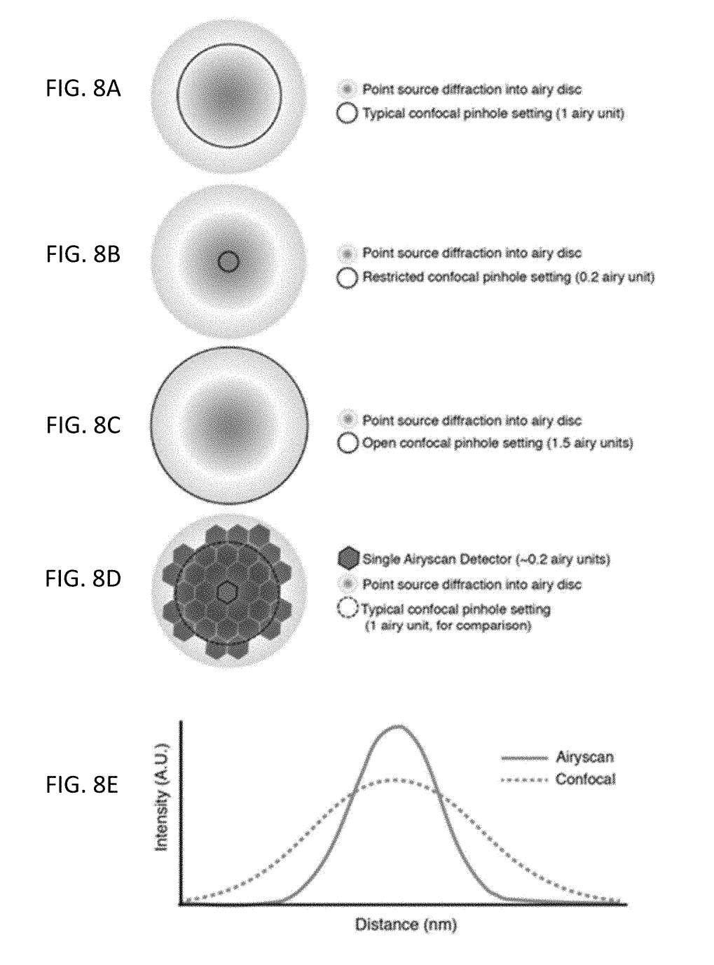

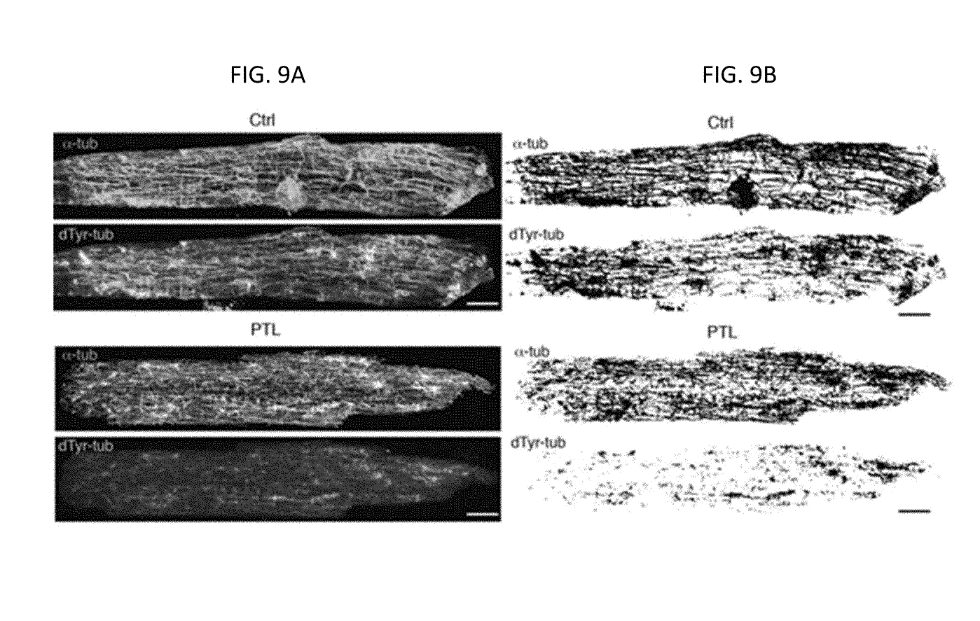

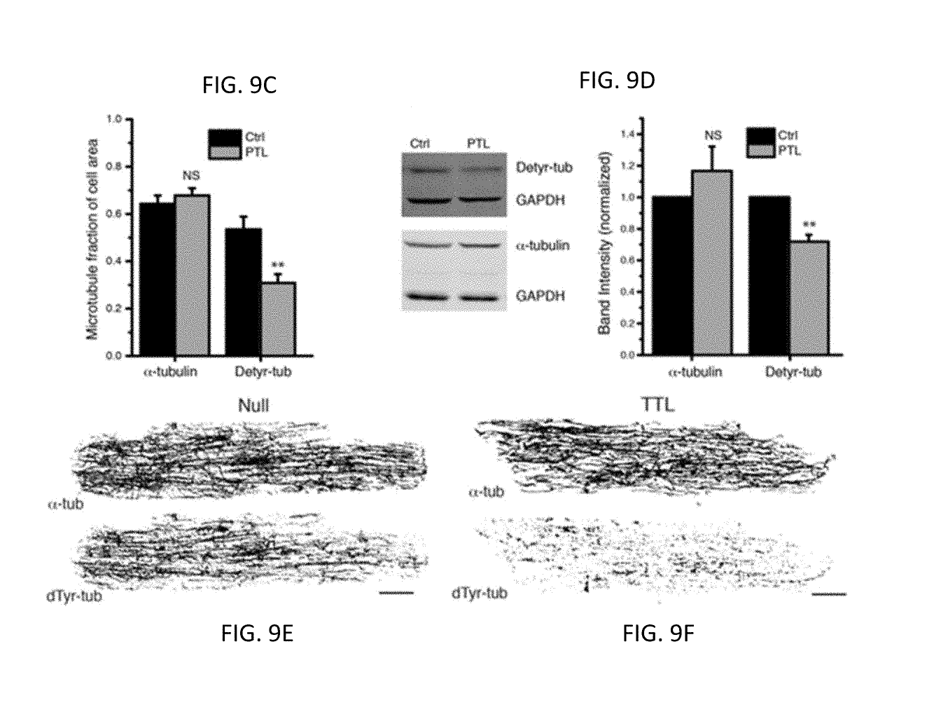

Confocal imaging was carried out on a Zeiss 780 laser scanning confocal scan head operating on an Axiovert Z1 inverted microscope equipped with a 40× oil 1.4 NA objective. High-speed super resolution imaging was carried out on a Zeiss 880 Airyscan confocal with a 63×1.4 oil NA objective (FIG. 8A-FIG. 8E, Airyscan application note can be found at http://www.zeiss.com/microscopy/enus/products/confocal-microscopes/Ism-880-with-airyscan-.html#downloads). Image analysis was performed using ZEN Black software for Airyscan processing, which involves signal integration from the 32 separate sub-resolution detectors in the Airyscan detector and subsequent deconvolution of this integrated signal (FIG. 8A-FIG. 8E). Additional signal processing was performed in Image J (NIH). For calculating the microtubule fraction of cell area (FIG. 2B-FIG. 2C), image analysis was performed blinded to the experimental treatment groups. A 1.5 μm max intensity projection was produced from 3 0.5 μm confocal z-sections. The threshold to determine microtubule positive pixels was determined from the average fluorescence of 3 background regions that clearly demonstrated no microtubule staining within the cell. From the resulting binary images (FIG. 9B), the microtubule positive fraction of the total cell area was calculated for alpha and detyrosinated tubulin (FIG. 2C).

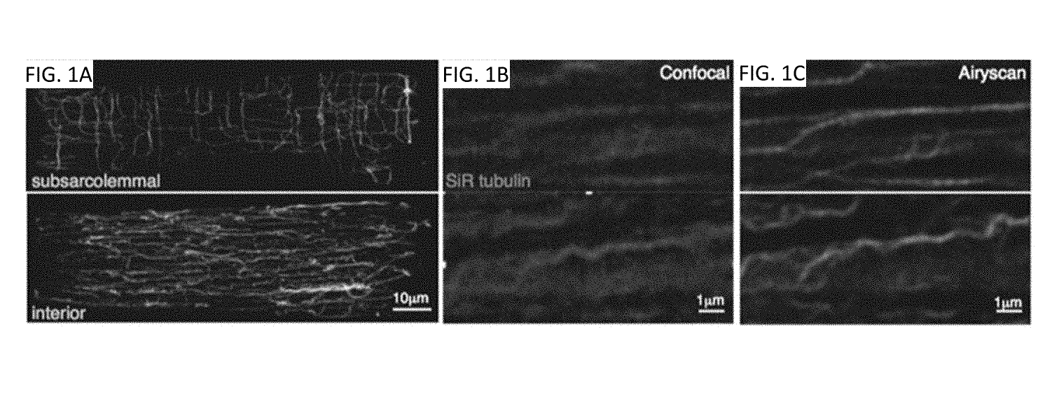

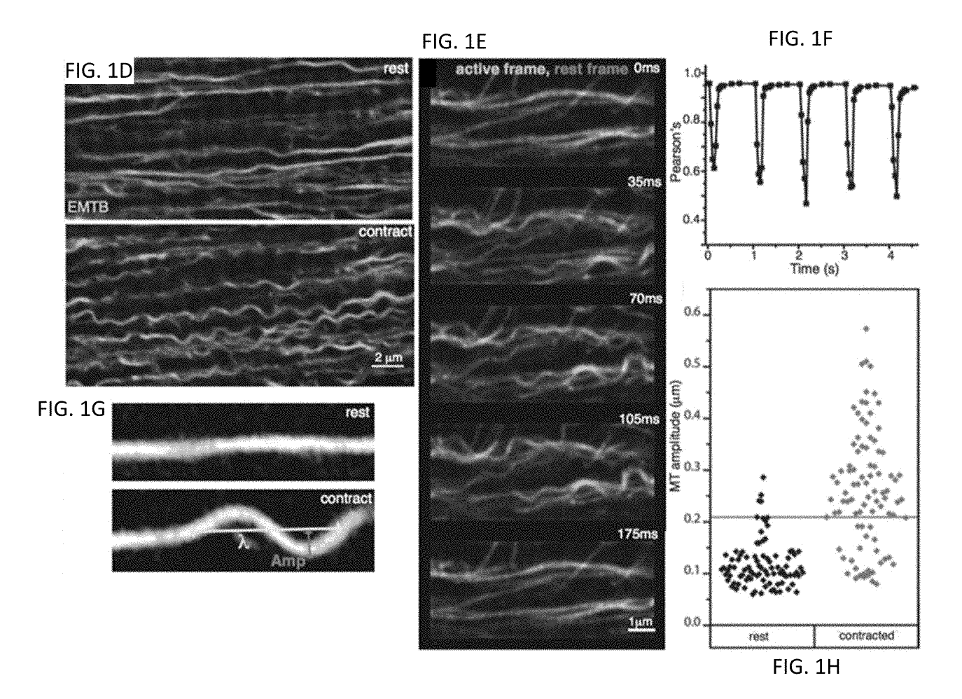

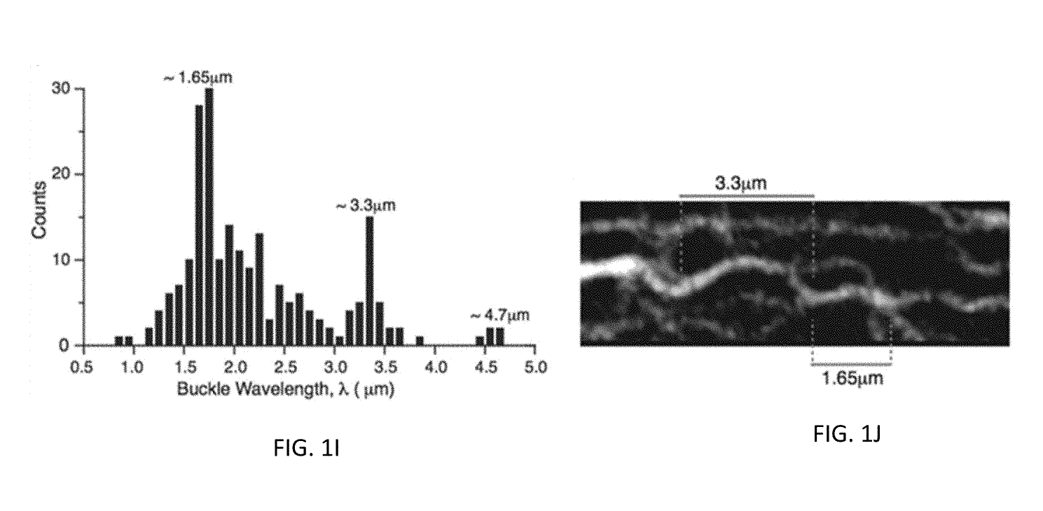

For images presented in the Example 1, adjustments for background using a 150 pixel radius rolling ball for confocal (with the exception of FIG. 1B, which were treated as Airyscan), or 50 pixel radius for Airyscan images, and clarity (0.5 Gaussian blur) were performed in ImageJ (http://rsb.info.nih.gov/ij/download.html). Brightness was increased in the confocal image in FIG. 1B for comparison of resolution with Airyscan. Colocalization analysis was performed using the JACoP plugin (http://rsb.info.nih.gov/ij/plugins/track/jacop.html). Pearson's coefficients were used to estimate colocalization between consecutive frames of the same microtubules (FIG. 1F); this parameter is optimal for estimating goodness of fit between two images, since it takes into account information from both bright and dark regions. Manders' coefficients were used to estimate fractional overlap of tubulin with desmin (FIG. 6E), as this parameter is focused entirely on the area which stained brightly for desmin, and provides a more quantitative comparison between tyrosinated and detyrosinated microtubules.

Analysis of buckling parameters was performed on 1-8 microtubules per image series. Analysis was done blind to the experimental condition and, where possible, followed a microtubule selected from resting frames to avoid biased selection of microtubules that buckled during contraction. A centerline was drawn along the axis of the microtubule and the maximum distance to the edge of the microtubule was recorded as the amplitude (FIG. 1G, red). MTs were further analyzed by recording the distance between 2 consecutive inflection points (FIG. 1G, yellow) as half the wavelength. This short metric was selected instead of full wavelength to maximize inclusion of microtubules for which labeling or z-displacement prevented inclusion of a full cycle in the image.

### Human Myocardial Tissue

Procurement of human myocardial tissue was performed under protocols approved by Institutional Review Boards at the University of Pennsylvania and the Gift-of-Life Donor Program (Pennsylvania, USA). Failing human hearts were procured at the time of orthotopic heart transplantation at the Hospital of University of Pennsylvania. Non-failing hearts were obtained at the time of organ donation from cadaveric donors. In all cases, hearts were arrested in situ using ice-cold cardioplegia solution, transported on wet ice, and flash frozen in liquid nitrogen within 4 hours of explantation. All samples were full-thickness biopsies obtained from the free wall of the left ventricle. Contractile parameters, including Left Ventricle Ejection Fraction (LVEF), were determined by echocardiography in patients.

### Data Handling and Statistics

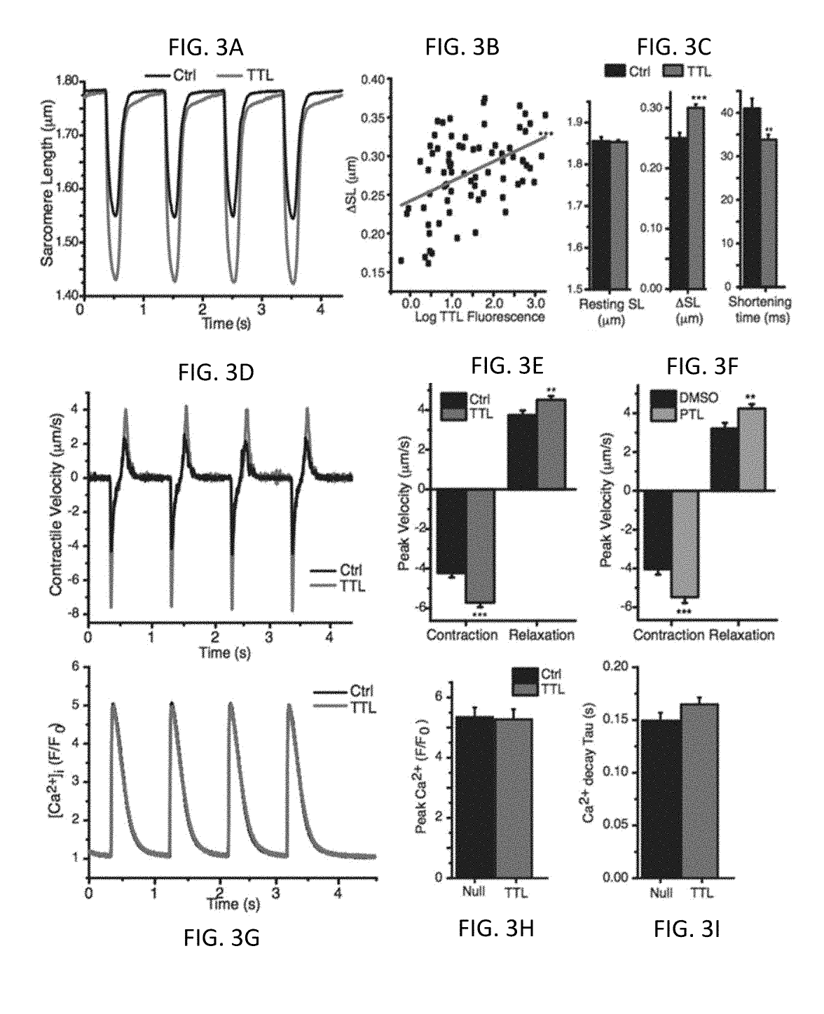

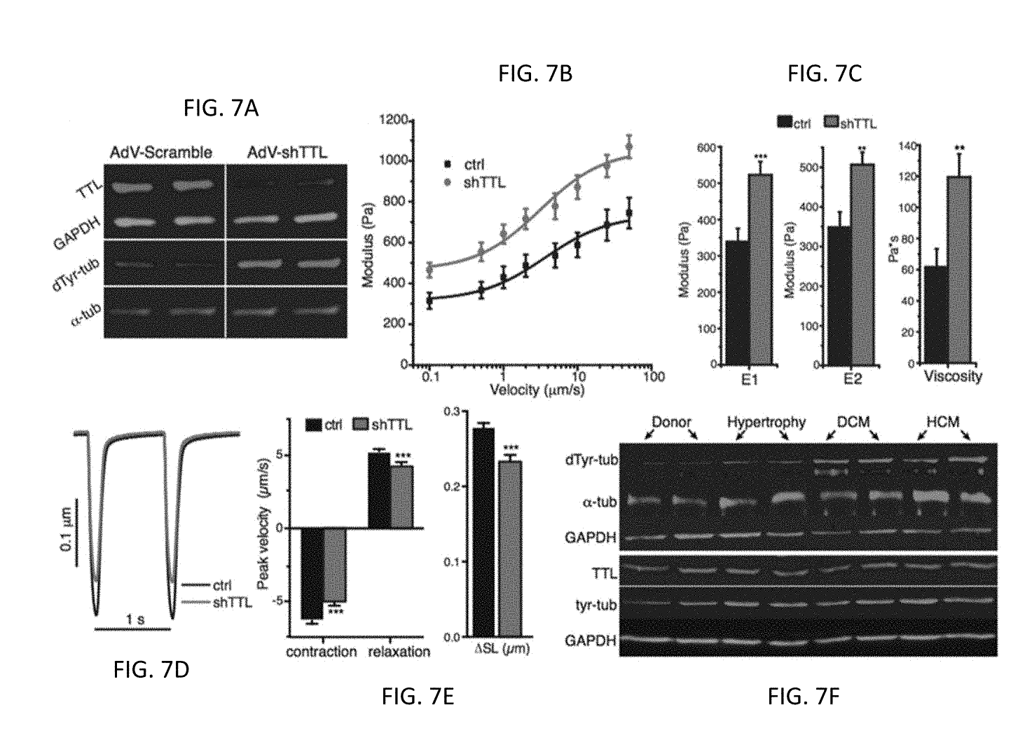

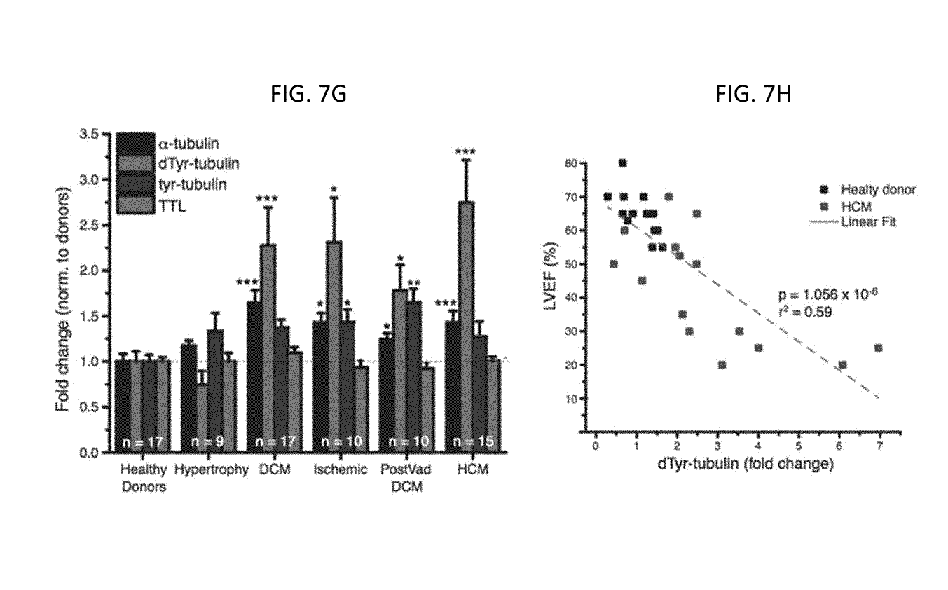

Single comparisons—Two-tailed Student's T-test was used when a single data set was compared to a single control condition (FIG. 2C; FIG. 3C, FIG. 3E, FIG. 3F, FIG. 3H, FIG. 3I; FIG. 4B; FIG. 6G; FIG. 7C, FIG. 7E). Where comparisons between sets were both repetitive and restricted, the Bonferroni multiple comparisons correction was used to adjust the significance threshold of T-tests accordingly (FIG. 4E; FIG. 7G). Fisher's exact test was used on proportional data (FIG. 2G, top).

Multiple comparisons—ANOVA with pairwise comparison by Tukey test was used when multiple data sets shared a single control condition (FIG. 2D, FIG. 2G bottom).

Fits of Standard Linear Solid State model (above) to pooled AFM data were constrained to the mean viscosity obtained from fits of individual cells (FIG. 4A; FIG. 6F; FIG. 7B). A linear fit was applied to the relationship of myocyte contractility vs. Log of TTL-dsRED fluorescence (FIG. 3B), as well as the relationships depicted in FIG. 6C and FIG. 7H. and slope determined to be significantly greater than 0 via ANOVA (p=1.2×10⁻⁵ for FIG. 3B, p=1.26×10-4 for FIG. 6C).

Outlier exclusion was applied to data exceeding 2 standard deviations.

Statistics are recorded in detail in Tables 1-4, 6, 8-9.

### Atomic Force Microscopy

The elastic modulus of isolated cardiomyocytes was measured using AFM indentation. Optical microscopy and AFM were performed simultaneously using an MFP3D AFM (Asylum Research) combined with an inverted Nikon TE-2000U microscope equipped with a Nikon 100×, 1.49 NA objective. TTL expression was determined by dsRED fluorescence captured via 532 nm laser excitation (CrystaLaser) and a CCD camera (Cascade-512B, Photometrics) at 900 ms exposure time controlled through the NIS Elements software package. All cells were observed with white light illumination from the MFP-3D during modulus characterization. Modulus characterization of myocytes was performed using 4.26 μm radius (R below) SiO₂ microparticle epoxied to SiN cantilevers with nominal spring constant 0.03 N/m (CP-PNPS-SiO-A, Nano And More, USA). Cantilever spring constants were manually calibrated in air by thermal vibrations using the MFP3D prior to each experiment using a clean glass substrate. Inverse optical lever sensitivity was determined in myocyte buffer solution prior to each round of measurements.

### Mathematical Model of Microtubule Buckling and Contractility

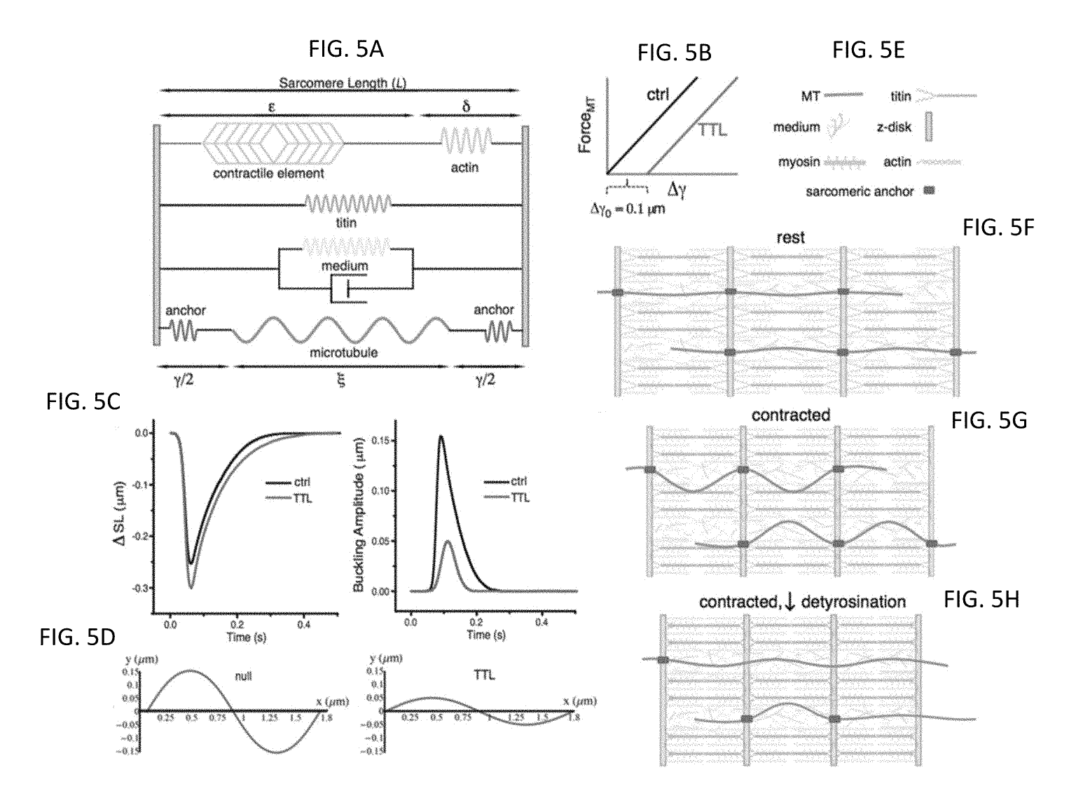

This single sarcomere model is based on the Hill model (38) that contains a contractile element, representing active acto-myosin contraction in series with an elastic element of the contractile arm (FIG. 1). MTs are placed in parallel to the contractile arm and connected to the z-disks via sarcomeric anchors. In addition the effect of titin is included with a parallel spring connected to the z-disks. The viscoelastic medium is represented by the Kelvin-Voigt viscoelastic model (spring and a dashpot in parallel) and is placed in parallel to the other mechanical elements (FIG. 5A). During the contraction of a sarcomere (equal to ΔL) with initial length L, myosin motors pull the actin filaments relative to each other and decrease the length of the sarcomeres by Δε, while the generated compressive force causes actin filaments to undergo a length change denoted by Δδ, which is minimal given the stiffness of actin (Table 5). Alternatively for the parallel branch, the compressive force acting on the MTs eventually leads to the buckling of the MTs and a decrease in the end-to-end length of the MTs that is denoted by Δξ. In addition, this compressive force causes a reduction in length (denoted by Δγ) of the two sarcomeric anchors present in the model (FIG. 5A).

By denoting the force generated by the actin-myosin contractile element with S, we can use Hill's law (38) to relate the rate of shortening of the actin-myosin element d(Δε)/dt to the force S

$$\frac{d ( Δɛ )}{dt} = b \frac{{S}_{0} - S}{a + S} \qquad ( 1 )$$

Here a and b are the coefficients that will be obtained by fitting the results of the model to the experimental data in the next section. According to Hill's law, the contraction rate of the actin-myosin contractile element decreases with increasing the applied force S, and finally reaches zero when S becomes equal to the stall force denoted by S₀.

As the actin filaments and the contractile elements are placed in series, the increase in the active force (S−S0) causes an elongation in the actin filaments (δΔ). Here by assuming linear elastic behavior for the deformation of actin, we can find this increase in the length of the actin filaments with force,

S−S₀=K₁Δδ (2)

Where K₁ is the elastic stiffness of the actin filament (as previously measured, 39).

The force acting on titin (P) can be related to its length change by using the following polynomial relationship obtained by fitting the force-elongation curve previously reported for a single titin molecule (40):

P−P₀=3(2×10²⁹ΔL⁶−2×10²³ΔL⁵+7×10¹⁶ΔL⁴−1×10¹⁰ΔL³+844.9ΔL²−2×10⁻⁵ΔL) (3)

Here P₀ is the initial force and the pre-factor 3 accounts for the presence of 2 groups of six parallel titin molecules in series across the sarcomere, as previously reported (41). Also in this equation, P and P₀ are in Newtons and ΔL is in meters.

Similarly, the force acting on the viscous medium (σ) can be related to the sarcomere length change by:

$$σ - {σ}_{0} = {K}_{2} Δ L + η {K}_{2} \frac{d ( Δ L )}{dt} \qquad ( 4 )$$

Where K₂ is the elastic stiffness of the medium, q is the relaxation time for the viscoelastic element and σ₀ is the initial force acting on the medium.

In addition, during the contraction of the sarcomeres, a compressive force (denoted by F) is exerted on the MT in the lower branch. Here by assuming linear elastic behavior for the deformation of the sarcomeric anchors with force,

$$F - {F}_{0} = \{ \begin{matrix}0 & Δγ < {Δγ}_{0} \\ {K}_{3} ( Δγ - {Δγ}_{0} ) & Δγ > {Δγ}_{0}\end{matrix} \qquad ( 5 )$$

where K₃ is the elastic stiffness of the sarcomeric anchors and F₀ is the initial force in the MT branch. Here we have defined a sarcomeric slip length Δγ₀ that represents the effect of the MT tyrosination with TTL overexpression. In control cells where roughly 70% of alpha tubulin is detyrosinated, and this is reduced by ˜80% due to TTL overexpression (see FIG. 2C). For TTL treated MTs the sarcomeres must shorten to some extent before engaging a detyrosinated portion of the MT via a sarcomeric anchor and applying force to the MT branch. Assuming a tubulin dimer spacing of ˜8 nm, this suggests a mean distance between detyrosinated subunits, and therefore a slip of ˜57 nm (8 nm/(0.7*0.2)) in TTL overexpressing myocytes. For null fibers, which are heavily detyrosinated, there is minimal slip and force is applied to the MT branch immediately upon contraction. For simplicity we assume Δγ₀=2×50 nm (factor 2 relates to the presence of two sarcomeric anchors in the sarcomere model) and Δγ₀=0 for the TTL and null treatments, respectively (FIG. 5B).

The MT in the lower branch fully resists a compressive force (F) that is smaller than the critical force (F_{cr}) required to buckle a MT. When the force exceeds the critical force (|F|>|F_{cr}|, note that for the compressive forces, the signs of the forces are negative), the decrease in the end-to-end length of the MT Δξ in response to F can be written as:

F=ψ(Δξ,Δξ) for |F|>|F_{cr}| (6)

Where ψ(Δξ,Δξ) is the function that relates the magnitude (Δξ) and velocity (Δξ) of the decrease in the end-to-end length of the MTs to the applied compressive force F and depends on the applied force, viscoelastic properties of the medium surrounding the MTs, as well as the flexural rigidity and length of the MTs.

The deformation in the constituents of the model are related to the overall contraction of the sarcomeres with:

$$\begin{matrix}\frac{d ( Δ L )}{dt} = \frac{d ( Δɛ )}{dt} + \frac{d ( Δδ )}{dt} & ( 7 ) \\ \frac{d ( Δ L )}{dt} = \frac{d ( Δξ )}{dt} + \frac{d ( Δγ )}{dt} & ( 8 )\end{matrix}$$

In order to simulate the cardiomyocyte contraction, external energy in the form of the log-normal pulse is input in the model, which approximates Ca²⁺ dependent activation of the myofilaments:

$$( S + F + P + σ ) ( L + Δ L ) - ( {S}_{0} + {F}_{0} + {σ}_{0} + {P}_{0} ) L = \frac{φ}{t} {e}^{- \frac{{( log ( t ) + {t}_{0} )}^{2}}{2 {ζ}^{2}}} \qquad ( 9 )$$

Here t₀ is the shift in the time to reach the maximum sarcomere contraction and the magnitude (ϕ<0) and duration (ζ and t₀) of the input pulse are the parameters that can be tuned in order to obtain the desired sarcomere contraction.

Equations 1-9 provide nine equations needed to find nine unknowns (ΔL, Δε, Δδ, Δξ, Δγ, F, P, σ and S), representing the displacements and forces applied to the individual elements in the model. Next, we derive the buckling-force relationship for the MTs of different lengths and embedded in the media with different viscoelastic properties and determine F=ψ(Δξ,Δξ) as defined in Eq. 6.

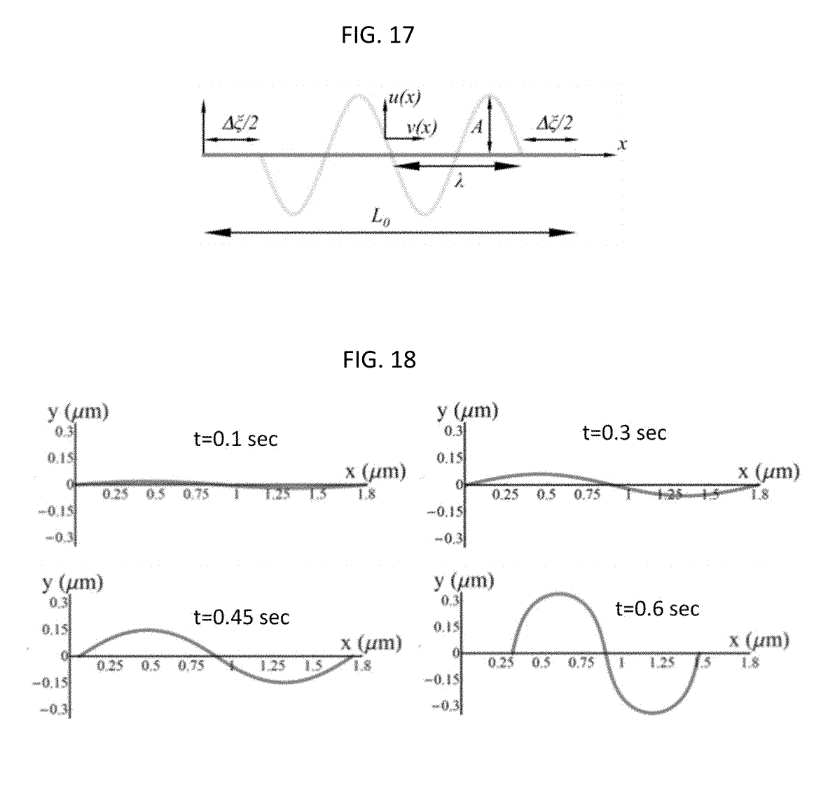

We next derive the shrinkage (Δξ)-force (F) relationship for the buckling of the MT that is surrounded by the viscoelastic medium representing the cytoplasm. As shown in Model FIG. 1, under a compressive force F, every point of the MT undergoes transverse displacement denoted by u(x) which is strictly restricted by the deformation of the surrounding medium. Here the amplitude and wavelength of the buckled shape are denoted by A and λ respectively. Also the coordinate system is placed at one end of the MT and the initial length of the MT is denoted by L₀ (FIG. 17).

By solving the Euler-Bernoulli beam equation, we can determine the amplitude and the wavelength of the buckled MT under an applied compressive force:

$$κ \frac{{d}^{4} u ( x , t )}{{dx}^{4}} - ( F - {F}_{0} ) \frac{{d}^{2} u ( x , t )}{{dx}^{2}} + β {u ( x , t )}^{3} = - α ( u ( x , t ) + η \dot{u} ( x , t ) ) \qquad ( 10 )$$

The first term in Eq. 10, represents bending force of the MT (with flexural rigidity κ=2×10⁻² Nm²) (6) and the second term corresponds to its axial deformation. The third term relates to the nonlinearity of the deformation with a coefficient factor that is denoted by β. On the right hand side, the applied force from the surrounding medium to the MT is written. Here α is the parameter that regulates the coupling of the deformation of the MT to the surrounding medium and can be estimated from the shear modulus of the surrounding medium as discussed in (6, 42):

$$α = \frac{4 π G}{ln ( λ \text{/} R )} \qquad ( 11 )$$

where λ≈1.8 μm is the characteristic wavelength, R≈12.5 nm is the radius of the MTs and G≈0.35 kPa is the shear modulus of the surrounding cytoplasm. Also for the purpose of this study, we use a fixed value of β=α₁.

In order to solve Eq. 10, we assume that the shape of the buckled MT is the composition of (m) different modes with sinusoidal form

$$u ( x , t ) = {\sum }_{i = 1}^{m} sin ( i \frac{π x}{{L}_{0}} ) {A}_{i} ( t ) \qquad ( 12 )$$

Where the amplitude of each mode Aᵢ(t) varies with time. With this definition, the wavelength of the buckled shape of each mode is Δ=2L₀/i.

By inputting Eq. 12 in Eq. 10, we use Galerkin's approximation to derive the ordinary differential equations necessary to obtain the amplitudes Aᵢ(t).

$${\int }_{x = 0}^{{L}_{0}} {( κ \frac{{d}^{4} u ( x , t )}{{dx}^{4}} - ( F - {F}_{0} ) \frac{{d}^{2} u ( x , t )}{{dx}^{2}} + β {u ( x , t )}^{3} + α ( u ( x , t ) + η \dot{u} ( x , t ) ) )}^{2} sin ( i \frac{π x}{{L}_{0}} ) dx = 0 i = 1 , 2 , … , m \qquad ( 13 )$$

Equation 13 provides differential equations needed to find amplitudes (Aᵢ(t)) of (m) different buckling shapes.

In order to find the decrease in the end-to-end length of the MT (Δξ) by buckling, here by equating the arc length of the buckled shape to the initial length of the straight MT, longitudinal and transverse displacements can be related as,

$$( L - 2 {x}_{0} ) + 2 v ( {x}_{0} , t ) = {\int }_{y = {x}_{0}}^{{L}_{0} - {x}_{0}} \sqrt{1 + {{u}^{′} ( y , t )}^{2}} dy \approx {\int }_{y = {x}_{0}}^{{L}_{0} - {x}_{0}} 1 + \frac{1}{2} {{u}^{′} ( y , t )}^{2} dy \approx {\int }_{x = {x}_{0}}^{{L}_{0} - {x}_{0}} \frac{1}{2} {{u}^{′} ( y , t )}^{2} dy + ( L - 2 {x}_{0} ) \qquad ( 14 )$$

Which is simplified to

$$v ( {x}_{0} , t ) \approx \frac{1}{4} {\int }_{x = {x}_{0}}^{{L}_{0} - {x}_{0}} {{u}^{′} ( y , t )}^{2} {dy}_{0} \qquad ( 15 )$$

and can be used to find the decrease in the end-to-end length of the MT (Δξ(t)=2ν(0, t)) with time.

The time dependence of MT buckling under a constant, sub-maximal force is shown in FIG. 18. Buckling amplitude increases with time inside the viscoelastic medium.

In response to a log-normal pulse stimulation (defined in Eq. 9), the sarcomeres contract and the generated compressive force eventually buckles the MTs. In order to produce the sarcomeric contraction of ˜0.25 μm observed in the experimental studies, we found the parameters listed in the table below by fitting the sarcomere length change obtained in the model to the experimental data. These parameters are obtained by assuming that the MTs are anchored to each consecutive z-disk and the initial length of the sarcomeres is L=1.8 μm. The comparison of the sarcomeric contraction with and without TTL treatment is shown in FIG. 5C.

As shown in FIG. 5C, in response to increasing the sliding distance between MT-sarcomere anchor points, the contraction of the sarcomeres increases by 0.05 μm (20%) in the TTL group compared to the null treatment. In this case, the resistance produced by the MTs to acto-myosin shortening is reduced and the contraction of the sarcomeres is increased, consistent with the experimental results (FIG. 3A-FIG. 3I). We can further examine how the engagement of the MTs resists shortening by comparing the buckled shape of the MTs in both cases. FIG. 5C-FIG. 5D show that the buckling (Δξ) of the MTs is strongly inhibited with TTL overexpression, as the buckling amplitude A is decreased, consistent with experimental data (FIG. 2I). This result also confirms that when the MTs are more prone to sliding on the z-disks in the TTL treatment group, they produce smaller resistance to sarcomeric contraction. This peculiar behavior of the MTs under null and TTL treatments is schematically shown in FIG. 5D.

The model results support the idea that buckling MTs can provide a physiologically relevant impediment to sarcomeric contraction. We estimate the MT buckling force to be ˜0.5 nN, similar to the value reported in (6), but likely higher due to the inclusion of viscous coupling of MTs to the surrounding environment present in our model. Counting the number of MTs in the representative cardiomyocyte in FIG. 1A (average intersections with 5 transverse lines), we calculate an average of 34.6+/−1.5 MTs in a 1.5 um thick slice. Extrapolating this value to the full depth of a myocyte (15 um), this predicts a total of ˜300-400 MTs at any given cross section. Given the buckling force of 0.5 nN for an individual MT, this equates to a total buckling force of ˜150-200 nN for the full cell. The total force generated by an electrically stimulated myocyte under our experimental conditions (at resting SL of ˜1.8 um and 1.8 mM external calcium) is ˜500-2000 nN. Thus, while both MT number and total force generation will vary from cell to cell, it is reasonable to expect that MT buckling could provide a resistive force equal to 5-40% of the total force generated by the myocyte. This value is large enough to be functionally relevant, but will not stall contraction.

MTs which buckle above threshold typically buckle with amplitude (A) of ˜0.2 um. We can thus estimate how the decrease in end-to-end distance of the MT compares to the total sarcomeric shortening as: (derived from Eq. 15)

$$2 v ( {x}_{0} = 0 , t ) \approx \frac{1}{2} {\int }_{x = 0}^{{L}_{0}} {{u}^{′} ( y , t )}^{2} {dy}_{0} = \frac{{A}^{2} {π}^{2}}{L} \qquad ( 16 )$$

Thus for A=0.2 um and L=1.8 um, the buckling of the MT accounts for a 0.22 um change in end-to-end distance. This represents ˜80-90% of the total observed sarcomere shortening (ΔSL=0.25 um, FIG. 3C). This is commensurate with the shortening expected if there are relatively rigid crosslinks at the Z-disk, and suggests that most of the sarcomere length change is taken up by the buckling of the MT itself, as opposed to the deformation of the anchoring complex between the MT and the sarcomere. This contrasts notably with TTL overexpressing cells, where, on the rarer occasions that MTs do buckle, a median buckling amplitude of ˜0.1 um can only account for ˜20% of the observed sarcomeric shortening (0.054 um change in end-to-end length compared to ΔSL=0.30 um, FIG. 3C), indicating that buckling cannot account for the majority of shortening in this case.

### Atomic Force Microscopy and Standard Linear Solid Model

Elastic moduli were determined by fitting the indentation curve to the Hertz model (Eq. 17) adjusted for a spherical indenter using the Asylum Research built in analysis software.

$$F ( δ ) = \frac{4}{3} \frac{E}{( 1 - {v}^{2} )} \sqrt{R} {\sqrt{δ}}^{3} \qquad ( 16 )$$

E is the elastic modulus, υ, is the cell Poisson ratio and δ is the indentation depth. Transverse compression of the cardiomyocyte is performed over a range of indentation speeds and the indentation speed is divided by the total indentation depth to obtain an effective indentation rate, f. Viscosity and velocity dependent elastic moduli were then derived by fitting each myocyte to a Standard Linear Solid Model:

$$E ( f ) = \frac{f η ( {E}_{1} + {E}_{2} ) + ( {E}_{1} {E}_{2} )}{{E}_{2} + f η} \qquad ( 18 )$$

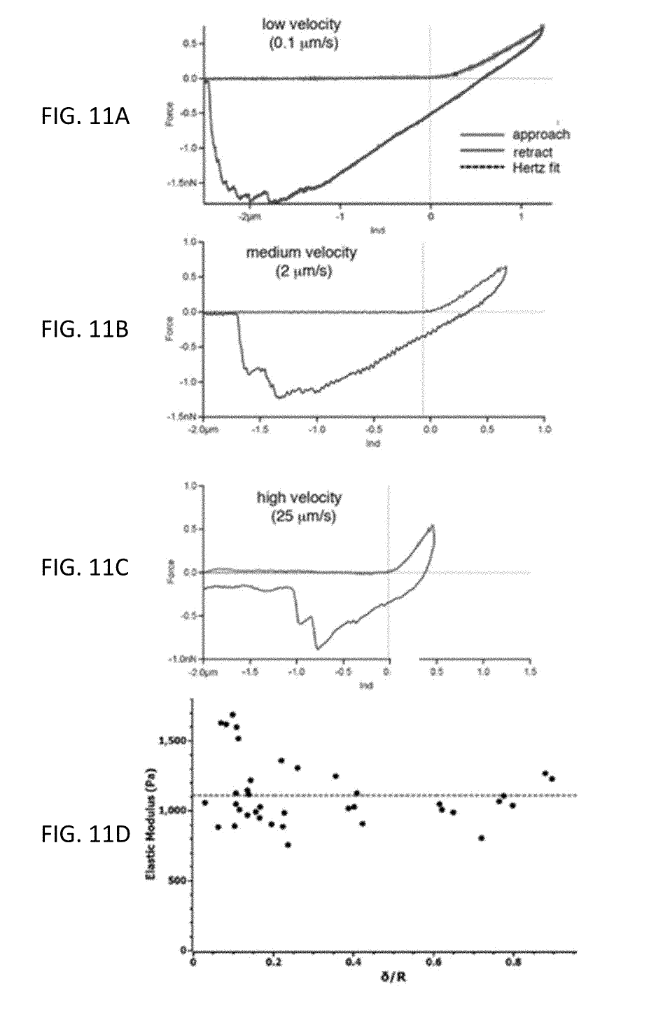

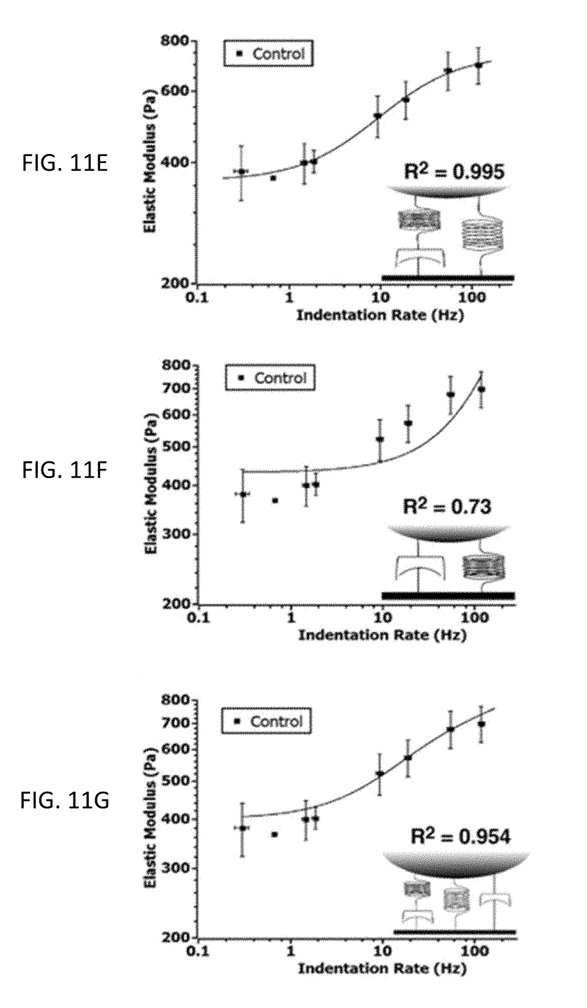

A detailed justification for the use of the Hertz and Standard Linear Solid Models and representative experimental traces are provided in FIG. 11A-FIG. 11G.

### Results

### Microtubules Buckle Under Contractile Load

Microtubule networks in cardiomyocytes have two major features (FIG. 1A): an orthogonal grid just beneath the membrane that wraps the myofibrils, and a deeper network composed primarily of longitudinal elements that interdigitate the myofibrils. Longitudinal MTs often run many sarcomeres in length, but do not span the full cell. Given that cardiomyocytes change shape during contraction, the MT cytoskeleton must accommodate this change. There are three apparent possibilities: MTs not anchored to other cytoskeletal or sarcomeric proteins could rearrange or slide passively with the surrounding medium; anchored MTs could directly experience contractile force and themselves deform under load; or the MTs could break/disassemble and reform. These possibilities offer divergent mechanisms for the regulation of mechano-signaling and the overall mechanics of the myocyte. Without direct observation, however, this behavior has been difficult to quantify.

Standard confocal imaging, while capable of resolving microtubules in living cells (18), suffers from limitations in signal to noise when pushed to speeds that can resolve events on the timescale of cardiomyocyte contraction (FIG. 1B). Consequently, we turned to a high-speed, sub-diffraction limit technique called Airyscan (see FIG. 8) (44). This technology maintains high signal to noise at the required temporal resolution, while offering a 1.7 fold improvement in spatial resolution beyond the standard diffraction limit.

Using the microtubule binding fluorogenic dye SiR tubulin (19) (FIG. 1C) we imaged internal microtubules during contractions triggered by 1 Hz electrical field stimulation in isolated cardiomyocytes. We were able to capture MT behavior during contraction, finding that longitudinally oriented MTs frequently deformed, developing sinusoidal buckles. Because SiR tubulin may polymerize MTs (19), we also generated adenovirus encoding a small fragment of the MT binding protein ensconsin fused to 3 copies of GFP (EMTB-3xGFP) to decorate MTs and achieved similar results (Table 1), but with improved signal to noise (FIG. 1D).

We measured blindly selected microtubules for deformation with two parameters —amplitude and wavelength (FIG. 1G). Where possible, the same MT was followed through contraction. Amplitude rose quickly from resting to contracted levels (FIG. 1H) with clearly visible buckles. Using a threshold of 2 standard deviations above resting amplitude, we found that two-thirds of MTs buckle under control conditions (FIG. 1H).

Microtubule buckles quickly reversed during relaxation, and the configuration of the MT network between contractions tightly colocalized with the network configuration from previous cycles (FIG. 1E-FIG. 1F), with a minimal mean reduction in Pearson's colocalization of 0.01 per contractile cycle (n=18 runs). The rapid and precise reversibility of the network deformations suggested tight coupling to the contractile apparatus and argues against microtubule breakage and regrowth contributing to mechanical properties and signaling over the timescale of myocyte contraction.

A notable feature of the MT buckles was the emergence of sub-populations of buckle wavelength centered at ˜1.65 μm, 3.3 μm and perhaps even 4.7 μm (FIG. 1I). These corresponded closely to the length of 1, 2 or 3 contracted sarcomeres, respectively. While MTs buckling outside of these populations could be found in our data without difficulty, these sub-populations were strongly indicative of ordered geometric constraints on the buckling MT. This was observed in certain cells where faint transverse staining at the Z-disk shows MTs buckling between sarcomeric constraints.

### Detyrosination Regulates Microtubule Buckling in the Heart

This robust buckling behavior of the microtubule network may be a result of a particularly high abundance of “detyrosinated” MTs in adult cardiomyocytes (20). Detyrosination is a PTM of α-tubulin where the c-terminal tyrosine residue has been cleaved by a tubulin carboxypeptidase (TCP); this process can be readily reversed by tubulin tyrosine ligase (TTL)(12). This tyrosination cycle is evolutionarily conserved across eukaryotes (21) and appears required for life (22), yet its functional roles are still poorly understood. Because detyrosination can protect MTs from disassembly (23, 24) and facilitate their cross-linking with intermediate filaments (IFs) (25, 26), we hypothesized that the high proportion of detyrosination may confer the resilient load-bearing capabilities of the cardiac cytoskeletal network.

Using antibodies specific to detyrosinated α-tubulin, we found a high abundance of detyrosination in the α-tubulin network of adult myocytes (FIG. 2A-FIG. 2B), as expected (13, 20). To test the role of detyrosinated MTs, we generated adenovirus encoding TTL (AdV-TTL) with a dsRed reporter. Expressing this construct in isolated cardiomyocytes could effectively reduce the level of detyrosination as shown by both immunofluorescence (FIG. 2A-FIG. 2B) and immunoblot (FIG. 2C-FIG. 2D), resulting in a 3-4 fold reduction in the amount of polymerized, detyrosinated MTs, with a concomitant upregulation of tyrosinated-tubulin (FIG. 2C-FIG. 2D, FIG. 9). Overexpression of TTL also resulted in a modest (10%) reduction in the density of the polymerized MT network (FIG. 2B), consistent with an increased disassembly of tyrosinated MTs (23, 24). We complemented this genetic strategy with a pharmacological approach to inhibit TCP using parthenolide (PTL)(27). PTL treatment also reduced the fraction of detyrosinated MTs, albeit to a lesser extent (2-fold) than AdV-TTL, and with no effect on MT network density (FIG. 9A-FIG. 9F).

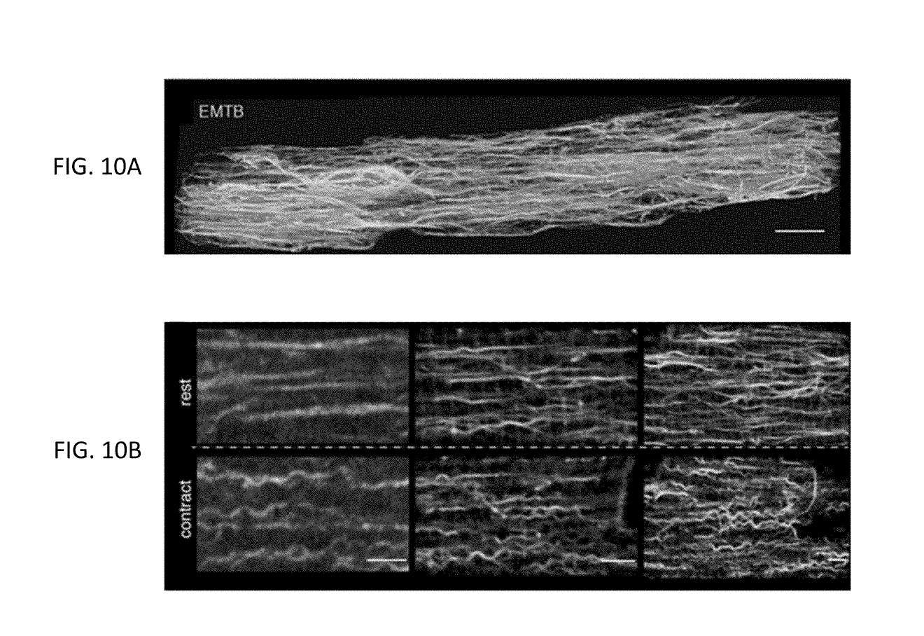

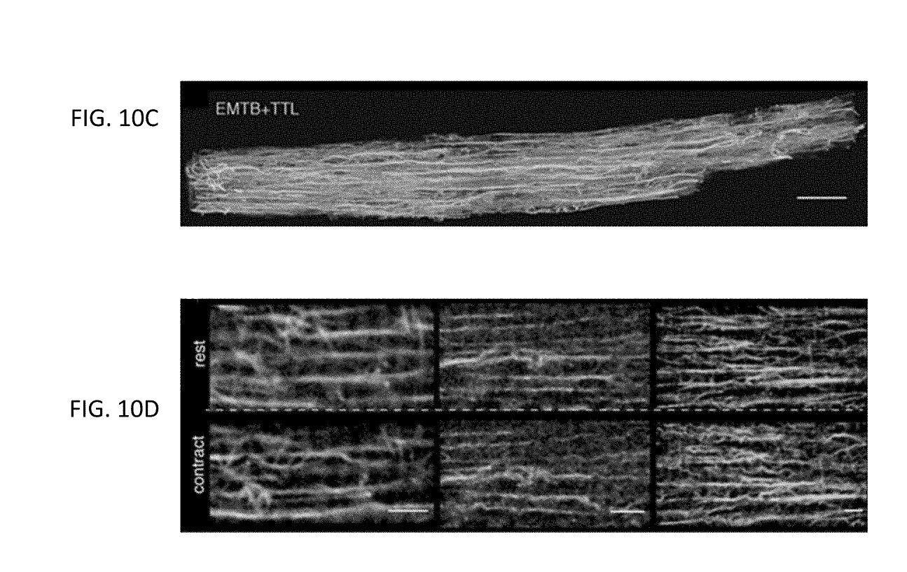

The load-bearing behavior of the microtubule network in cardiomyocytes overexpressing TTL or treated with PTL was dramatically different from control myocytes. Tyrosinated-MTs frequently seemed to simply slide in the moving cell (FIG. 2E-FIG. 2F), orange arrows; FIG. 10A-FIG. 10D), rather than buckling under load. This behavior was again reversible, with a minimum reduction in Pearson's colocalization over successive contractions that was not different from controls (p=0.87, n=19 runs). The occurrence of buckling in TTL overexpressing and PTL treated cells fell significantly (FIG. 2G left), while amplitude changes observed on the same MT between rest and contraction also dropped significantly (FIG. 2G, right, Table 1).

TABLE 1
Statistical parameters of buckling, including those presented in FIG. 2

| Immuno- | | | | | | | | | |
|---|---|---|---|---|---|---|---|---|---|
| fluoresence | Null | | TTL | | Ctrl | | PTL | | Test |
| N (cells) | 14 | | 13 | | 4 | | 6 | | |
| α-tub (% cell area) | 65.4 | (2.4) | 55.1 | (2.8) | 64.3 | (3.6) | 67.8 | (3.1) | |
| P (vs null/ctrl) | | | 0.01 | | | | 0.47 | | T-test |
| Detyr (% cell area) | 43.9 | (3.5) | 12.9 | (1.52) | 53.5 | (5.4) | 30.8 | (3.8) | |

| P (vs null/Ctrl) | | | 2.33 × 10⁻⁹ | | | | 0.007 | T-Test | |
|---|---|---|---|---|---|---|---|---|---|
| Western Blotting | Null | | TTL (0.01) | | TTL (0.1) | | Test | | |
| N (cell lysates) | 6 | | 6 | | 6 | | | | |
| TTL | 1.0 | (0.07) | 9.26 | (3.05) | 25.03 | (2.22) | | | |
| P (vs null) | | | 0.059 | | 7.1 × 10⁻⁵ | | Tukey | | |
| | | | | | | | ANOVA | | |
| α-tubulin | 1.0 | (0.07) | 1.27 | (0.09) | 1.00 | (0.16) | | | |
| P (vs null) | | | 0.25 | | 1 | | Tukey | | |
| | | | | | | | ANOVA | | |
| detyr Tubulin | 1.0 | (0.04) | 0.74 | (0.06) | 0.28 | (0.03) | | | |
| P (vs null) | | | 0.003 | | 9.08 × 10⁻⁶ | | Tukey | | |
| | | | | | | | ANOVA | | |
| tyr Tubulin | 1.0 | (.05) | 2.63 | (.022) | 3.32 | (.076) | | | |
| P (vs null) | | | 0.14 | | 0.02 | | Tukey | | |
| | | | | | | | ANOVA | | |
| | | | | | | | | SiR | |
| Buckling | EMTB | | Parth + EMTB | | TTL + EMTB | | Test | Tubulin | |
| N (microtubules) | 90 | | 24 | | 62 | | | 22 | |
| N Buckled | 60 | | 9 | | 15 | | | 12 | |
| P (vs EMTB) | | | 0.01742 | | 2.48 × 10⁻⁷ | | Fisher's Exact | — | |
| Amp Rest | 0.117 | (0.004) | 0.134 | (0.009) | 0.113 | (0.005) | | 0.141 | (0.021) |
| P (vs EMTB) | | | 0.24279 | | 0.83686 | | Tukey | — | |
| | | | | | | | ANOVA | | |
| Amp Contract | 0.261 | (0.011) | 0.212 | (0.021) | 0.173 | (0.067) | | 0.269 | (0.03) |
| P (vs control) | | | 0.08354 | | 4.80 × 10⁻⁷ | | Tukey | — | |
| | | | | | | | ANOVA | | |
| P (vs rest) | 3.32 × 10⁻²² | | 0.00125 | | 2.89 × 10⁻⁸ | | T-test | — | |
| N (followed) | 73 | | 24 | | 41 | | | 22 | |
| Amp (Δ) | 0.141 | (0.011) | 0.078 | (0.016) | 0.055 | (0.01) | | 0.129 | (0.032) |

| P (vs con) | | | 7.76 × 10⁻³ | 3.46 × 10⁻⁶ | | Tukey | — |
|---|---|---|---|---|---|---|---|
| | | | | | | ANOVA | |
| N (λ) | 216 | | | 138 | | | |
| Λ | 2.20 | (0.05) | | 2.11 | (.05) | | |

| | | | |
|---|---|---|---|
| P (vs EMTB) | 0.225 | T-test | — |

When MT buckling was observed, the mean wavelength was not significantly different between control and TTL overexpressing cells (Table 1). However, the majority of MTs in TTL overexpressing myocytes no longer buckled at the wavelength of a single sarcomere, and no sub-populations at multiples of the sarcomeric period were observed (FIG. 2H). Instead, the majority of these MTs buckled in a single population at wavelengths between 2-3 m, suggesting that MT buckling was less constrained by a sarcomeric interaction after detyrosination was reduced (FIG. 2I).

### Detyrosinated Microtubules Resist Contractile Compression

The energy required to deform detyrosinated microtubules under compressive load could confer some meaningful resistance to myocyte contraction. We thus tested directly if MT detyrosination affects contractility in beating cardiomyocytes. Following overexpression of TTL, we found significant enhancements in both the magnitude (FIG. 3A-FIG. 3C) and peak rate (FIG. 3D-FIG. 3E) of sarcomere shortening (Table 2).

PTL had a similar effect on contractility (FIG. 3F, Table 2). Peak relaxation rates were also increased, which could be due to a decrease in cellular viscosity (FIG. 4B), or may reflect the increased magnitude of shortening and therefore compression of internal elastic elements (e.g. titin) that develop restoring force (28). These contractile changes were not associated with any significant difference in global calcium transients (FIG. 3G-FIG. 3I) or resting sarcomere length (FIG. 3C), suggesting a change in intrinsic mechanical resistance associated with the ability of detyrosinated MTs to bear compressive load.

TABLE 2

| Contractility | Adv-Null | | AdV-TTL | | Ctrl | | PTL | | Test |
|---|---|---|---|---|---|---|---|---|---|
| N (cells) | 46 | | 45 | | 33 | | 27 | | |
| Resting SL (μm) | 1.855 | (0.01) | 1.853 | (0.01 | 1.804 | (0.005) | 1.803 | (0.008) | |
| P (vs null/dmso) | | | 0.865 | | | | 0.869 | | T-test |
| ΔSL (μm) | 0.250 | (0.009) | 0.300 | (0.006) | 0.165 | (0.012) | 0.201 | (0.011 | |
| P (vs null/dmso) | | | 2.15 × 10⁻⁵ | | | | 0.030 | | T-test |
| Vmax (μm/s) | −4.22 | (0.21) | −5.72 | (0.22) | −4.09 | (0.31) | −5.35 | (0.33) | |
| P (vs null/dmso) | | | 5.59 × 10⁻⁶ | | | | 0.007 | | T-test |
| Vmin (μm/s) | 3.74 | (0.22) | 4.51 | (0.19) | 3.27 | (0.31) | 4.28 | (0.25) | |

| P (vs null/dmso) | | | 0.011 | | | 0.016 | T-test |
|---|---|---|---|---|---|---|---|
| Shortening time | 41.0 | (2.2) | 33.9 | (1.1) | | | |
| (ms) | | | | | | | |
| P (vs null) | | | 0.006 | | | | |
| Relaxation time | 226 | (6.5) | 216 | (5.8) | | | |
| (ms) | | | | | | | |
| P (vs null) | | | 0.252 | | | | |
| Calcium | | | | | | | |
| Transient | Null | | TTL | | Test | | |
| N (cells) | 25 | | 23 | | | | |
| F/F0 | 5.34 | (0.33) | 5.27 | (0.34) | | | |
| P (vs. null) | | | 0.87 | | T-test | | |
| Decay Tau | 0.15 | (0.01) | 0.16 | (0.01) | | | |

### Detyrosination Regulates Myocyte Mechanical Properties

We next measured mechanical resistance directly using atomic force microscopy (AFM). AFM measurements of transverse stiffness were performed across a range of indentation rates. Myocyte stiffness changed significantly with indentation rate and was well fit by a standard linear solid model (SLSM, Methods, FIG. 11A-FIG. 11G, FIG. 4A). At low rates (100 nm/s), the stiffness of the cardiomyocyte was essentially elastic, reported as E1, and was reduced by PTL treatment and TTL overexpression (FIG. 4B). At higher rates the modulus increased by E2, which reflects cross-linked material inside the cell that slips on the timescale of slower measurements, but “turns on” (stiffens) at faster timescales (>2 μm/s) (29). The viscosity derived by the SLSM defines the rate above which these cross-links are engaged. TTL overexpression significantly decreased E2 and viscosity (FIG. 4B), suggesting that reducing detyrosination decreases the number of cross-links engaged at physiological strain rates in the cardiomyocyte.

TABLE 3
Statistical parameters of AFM, including
those presented in FIG. 4A-FIG. 4E.

| Visco- | | | | | |
|---|---|---|---|---|---|
| elastic | | | | | |
| Param- | | | | | |
| eters | Null | TTL | Ctrl | PTL | Test |
| N | 13 | 14 | 11 | 12 | |
| (cells) | | | | | |
| E₁ (Pa) | 393.8(59.5) | 191.6(29.0) | 300.4(33.2) | 170.6(22.5) | |
| P (vs | | 0.004 | | 0.004 | T-test |
| null/ | | | | | |
| ctrl) | | | | | |
| E₂ (Pa) | 294.7(32.1) | 130.4(23.1) | 293.1(31.5) | 215.3(25.0) | |
| P (vs | | 2.95 × 10⁻⁴ | | 0.064 | T-test |
| null/ | | | | | |
| ctrl) | | | | | |
| η | 84.2(13.0) | 17.3(3.4) | 68.2(8.4) | 38.8(8.0) | |
| (Pa*s) | | | | | |
| P (vs | | 2.55 × 10⁻⁵ | | 0.019 | T-test |
| null/ | | | | | |
| ctrl) | | | | | |
| SLSM | 0.92 | 0.88 | 0.91 | 0.91 | |
| Fit r² | | | | | |

TABLE 4
Statistical parameters of cardiomyocyte stretch, including those presented in FIG. 4A-FIG. 4E.

| Stretch | Null | TTL | Null | TTL | Null | TTL | Null | TTL | Null | TTL | Null | TTL | |
|---|---|---|---|---|---|---|---|---|---|---|---|---|---|
| (μm) | (0) | (0) | (4) | (4) | (8) | (8) | (12) | (12) | (16) | (16) | (20) | (20) | Test |
| N | 15 | 21 | | | | | | | | | | | |
| (cells) | | | | | | | | | | | | | |
| ΔSL | 0 | 0 | 0.058 | 0.086 | 0.105 | 0.159 | 0.161 | 0.252 | 0.217 | 0.328 | 0.265 | 0.398 | |
| (μm) | | | (0.012) | (0.013) | (0.014) | (0.015) | (0.016) | (0.014) | (0.016) | (0.023) | (0.019) | (0.03) | |
| P (vs | | | | | | | | | | | | | RM |
| null) | | | | | | | | | | | | | ANOVA |
| Fpeak | 0 | 0 | 0.0771 | 0.0567 | 0.140 | 0.0872 | 0.234 | 0.144 | 0.322 | 0.200 | 0.386 | 0.248 | |
| (μN) | | | (0.012) | (0.015) | (0.012) | (0.015) | (0.012) | (0.015) | (0.012) | (0.015) | (0.012) | (0.015) | |
| P (vs | | | | | | | | | | | | 7.4 × 10⁻⁷ | RM |
| null) | | | | | | | | | | | | | ANOVA |
| Fs.s. | 0 | 0 | 0.0525 | 0.0487 | 0.0728 | 0.0667 | 0.115 | 0.0884 | 0.162 | 0.120 | 0.183 | 0.167 | |
| (μN) | | | (0.010) | (0.011) | (0.010) | (0.011) | (0.010) | (0.011) | (0.010) | (0.011) | (0.010) | (0.011) | |
| P (vs | | | | | | | | | | | | 0.023 | RM |
| null) | | | | | | | | | | | | | ANOVA |

| Binned | Null | TTL | Null | TTL | Null | TTL | Null | TTL | Null | TTL | |
|---|---|---|---|---|---|---|---|---|---|---|---|
| L-T | (1.8) | (1.8) | (1.9) | (1.9) | (2.0) | (2.0) | (2.1) | (2.1) | (2.2) | (2.2) | Test |
| SL | 1.82 | 1.80 | 1.91 | 1.91 | 2.0 | 2.0 | 2.10 | 2.10 | 2.19 | 2.24 | |
| (μm) | (0.01) | (0.02) | (0.01) | (0.01) | (0.01) | (0.01) | (0.01) | (0.01) | (0.01) | (0.01) | |
| Fpeak | 0.029 | 0 | 0.087 | 0.057 | 0.224 | 0.150 | 0.311 | 0.200 | 0.387 | 0.235 | |
| (μN) | (0.02) | (0) | (0.018) | (0.01) | (0.029) | (0.016) | (0.019) | (0.021) | (0.054) | (0.027) | |
| P (vs | | | | 0.15 | | 0.023 | | 4.2 × 10⁻⁴ | | 0.018 | T-test, |
| null) | | | | | | | | | | | Bonferroni |
| Fs.s. | 0.028 | 0 | 0.053 | 0.039 | 0.119 | 0.096 | 0.15 | 0.10 | 0.187 | 0.143 | 5 |
| (μN) | (0.02) | (0) | (0.011) | (0.007) | (0.015) | (0.009) | (0.01) | (0.008) | (0.021) | (0.015) | |
| P (vs | | | | 0.299 | | 0.178 | | 8.3 × 10⁻⁴ | | 0.179 | T-test, |
| null) | | | | | | | | | | | Bonferroni |

The fact that microtubules deform under load and resist sarcomere shortening implies a transfer of force between MTs and the sarcomere. If MTs resist longitudinal compression, they could also confer a tensile resistance when the sarcomeres are stretched, as occurs during diastolic filling. To test this we measured passive stiffness directly along the longitudinal axis of TTL overexpressing myocytes. We attached cardiomyocytes to laser-etched cell holders (FIG. 4C, FIG. 12A-FIG. 12G) via a biological adhesive (1) and subjected them to step-like changes in length, while simultaneously measuring sarcomere length and passive force with a high-sensitivity transducer (FIG. 4D). A typical force response (FIG. 4D) showed a rapid rise to peak force during the high velocity stretch (Fₚₑₐₖ), containing both elastic and viscous elements, followed by a relaxation to a steady-state force (F_{s,s}) that largely reflects the elastic stiffness of the myocyte. For a given step size, TTL overexpressing myocytes exerted significantly reduced peak forces during physiological length changes, with modest reductions in steady state force (FIG. 12F). The TTL overexpressing cells also underwent significantly larger changes in sarcomere length with any given step (FIG. 12G), indicating increased sarcomere compliance, and suggesting that stiffer sarcomeres in control cells distribute the length change to other compliant components in series. As can be surmised by FIG. 12F and FIG. 12G, TTL overexpression decreased tension across the physiological range of sarcomere lengths achieved during diastolic filling (FIG. 4E), indicating a role for detyrosinated microtubules as tensile resistant elements. Visual evidence supporting such a relationship was seen in MT networks in a control cell at resting and stretched length (FIG. 12A-FIG. 12G, FIG. 4C). At resting length there was some inherent slack in the MT network, while the same MTs became taut when the cell was stretched and held at long sarcomere lengths.

### Model of Microtubule Contribution to Cardiac Contractility

TABLE 5
Modeling Parameters.

| Param- | | | | |
|---|---|---|---|---|
| eter | Value | | Meaning | Source |
| L | 1.8 | μm | Initial sarcomere | Contractility |
| | | | length | Data |
| Δ L | 0.25 | μm | Change in SL | Contractility |
| | | | | Data |
| K₁ | 65.3 | pN/nm | Stiffness of | Literature(Kojima |
| | | | contractile element | 94) |
| γ₀ | 100 | nm | Slide distance of | Detyrosination |
| | | | MT anchor on TTL | levels |
| | | | treated MT | |
| κ | 2 × 10−²³ | Nm² | Flexural rigidity | Literature (9) |
| | | | of MT | |
| m | 3 | | Maximum number | — |
| | | | of modes considered | |
| η | 0.1 | sec | Viscoelastic time- | AFM Data |
| | | | scale of medium | |
| b | −5 | μm/sec | Peak contractile | Contractility |
| | | | velocity | Data |
| G | 0.35 | kPa | Shear modulus | AFM Data |
| K₂ | 0.43 | pN/nm | Elastic stiffness | Fit |
| | | | of medium | |
| K₃ | 5.7 | pN/nm | Elastic stiffness | Fit |
| | | | of MT anchors | |
| S₀ | 1.55 | nN | Initial stall force | Fit |
| F₀, σ₀, | 4 | nN | Initial force acting | Fit |
| P₀ | | | on parallel arms | |
| A | 0.7 | nN | Coefficient of | Fit |
| | | | thermal loss | |
| Φ | −1.25 | nN μm sec | Magnitude constant | Fit |
| | | | of input energy | |
| Z | 0.1 | sec | Lognormal time | Fit |
| | | | parameter | |
| t₀ | 1.9 | sec | Lognormal time | Fit |
| | | | parameter | |

| | | | |
|---|---|---|---|
| Δ ε | Variable | Active contraction | Eq. 1, 7 |
| | | displacement | |
| Δ δ | Variable | Contractile element | Eq. 2, 7 |
| | | axial displacement | |
| Δ ξ | Variable | Microtubule axial | Eq. 6, 8 |
| | | displacement | |
| Δ γ | Variable | Axial displacement | Eq. 5, 8 |
| | | of 2 MT anchors | |
| u | Variable | Lateral (y) | Eq. 10, 12, 13, |
| | | displacement of MT | 14, 15 |
| | | on long axis (x) | |
| A | Variable | Amplitude of | Eq. 12 |
| | | energetically | |
| | | optimal mode | |
| Aᵢ | Variable | Buckling amplitude | Eq. 12 |
| | | of mode i | |
| v | Variable | Longitudinal (x) | Eq. 14, 15 |
| | | displacement of the | |
| | | MT | |
| S | Variable | Force on contractile | Eq. 1, 2, 9 |
| | | arm | |
| F | Variable | Force on MT arm | Eq. 5, 6, 9 |
| σ | Variable | Force on | Eq. 4, 9 |
| | | viscoelastic | |
| | | medium arm | |
| P | Variable | Force on Titin arm | Eq. 3, 9 |

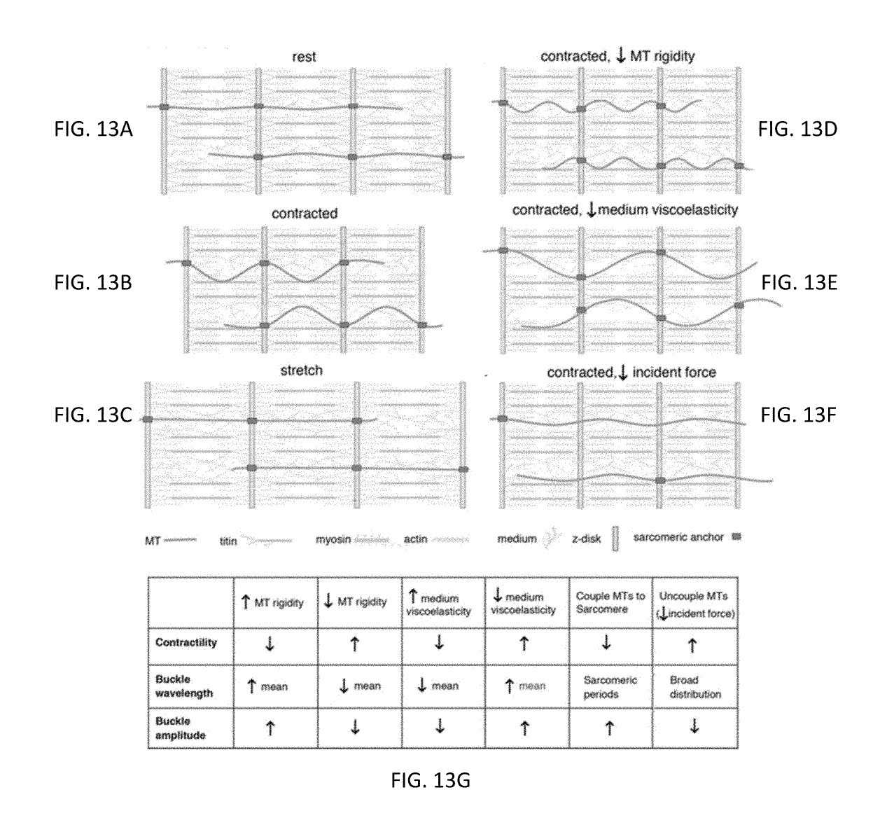

We next sought to develop a mathematical model to recapitulate the experimentally measured changes in MT buckling and contractility when detyrosination is reduced. Previous work modeling microtubule buckles (6) suggests that three critical variables determine buckling behavior; MT stiffness, stiffness of the surrounding medium, and force incident on the long axis of the MT. How these three variables are predicted to alter MT behavior and myocyte mechanics is described in FIG. 13A-FIG. 13G. Of the three, only a decrease in incident force can explain our experimental observations after suppressing detyrosination. If MT anchoring to the sarcomere is disrupted, the reduced incident force on the MT may drop below the critical force required for buckling, resulting in simultaneous decreases in buckling amplitude (FIG. 2A-FIG. 2I) and viscoelasticity (FIG. 3A-FIG. 3I, FIG. 4A-FIG. 4E). The sarcomeric periodicity of buckles (FIG. 1I) also suggests an underlying structural constraint that changes in MT or medium stiffness alone cannot explain. We thus chose to model MT buckling within a contractile model that includes a MT compression resistive element whose interaction with the sarcomere can be varied (see Model for details).

Using the mechanical scheme detailed in FIG. 5A, we fitted the contraction resulting from a log-normal force input to derive both contractile and buckling parameters. By modifying the incident force applied to a MT for a given amount of sarcomere shortening (FIG. 5A, Model), we simulated the effect of a sarcomeric anchor sliding and then catching at detyrosinated regions of the MT. Inclusion of a 100 nm slide (50 nm at each anchor, see Model) before MTs engage with the rest of the sarcomere is reasonable given the approximately 80% reduction in detyrosinated area observed by immunofluorescence with TTL overexpression (FIG. 2C) and reflects the fact that reductions in detyrosination would increase the average distance between detyrosinated tubulins that could interact strongly with MT anchoring points. This disruption of MT-sarcomere coupling produced model outputs (FIG. 5C-FIG. 5D) that closely recapitulated our experimental contractility and buckling results.

An alternate possibility to the sliding anchor is that the anchor is completely uncoupled by suppressing detyrosination, reverting to buckling behavior governed by local viscoelasticity rather than underlying structure, as proposed for less rigidly organized cell types including developing myocytes (6). In either case the coupling of MTs to the sarcomere is reduced, impairing their ability to resist contraction.

### Potential Role for Desmin as a Sarcomeric Microtubule Anchor

The putative characteristics of the anchor—a mechanically stiff protein, capable of complexing with microtubules and restricted to a spatially defined region of the sarcomere-suggested the intermediate filament desmin as an immediate candidate. Desmin forms structural bundles that complex with the z-disk (30), and intermediate filaments can form detyrosination-dependent cross-links with MTs (31, 32).

We first sought to determine if desmin preferentially associates with detyrosinated MTs. Co-sedimentation of cardiomyocyte lysates showed that desmin pellets with polymerized MTs (FIG. 6A) in direct proportion to their level of detyrosination (FIG. 6B-FIG. 6C), indicating a specific and sensitive interaction. We also co-stained cardiomyocytes from desmin knockout (KO) and WT mice for desmin and both tyrosinated and detyrosinated tubulin to observe any preferential interaction. The two populations of MTs show similar overall patterning in WT myocytes, except for a specific accumulation of detyrosinated (and not tyrosinated) tubulin in transverse bands at the z-disk that co-localized with desmin (FIG. 6D-FIG. 6E, FIG. 14A-FIG. 14H). Strikingly, KO animals lacked this transverse pattern completely (FIG. 6D, FIG. 14H), although the z-disk itself remained intact (FIG. 14C). In addition, KO myocytes possessed a denser (FIG. 14F) and more disorganized MT network (FIG. 6D, FIG. 14B and FIG. 14E), suggesting that desmin is required for proper MT network organization.

If desmin cross-links with detyrosinated MTs to structurally reinforce the network, then the removal of desmin should both decrease cytoskeletal stiffness and prevent tyrosination-dependent changes in viscoelasticity. Blind studies in WT and KO myocytes revealed that desmin KO myocytes were significantly less stiff than WT counterparts, and that treatment with PTL no longer reduced viscoelasticity (FIG. 6F-FIG. 6G).

TABLE 6
Statistical parameters of AFM for desmin data presented in FIG. 6A-FIG. 6G

| Viscoelastic | | | | | | | | | |
|---|---|---|---|---|---|---|---|---|---|
| Parameters | WT | | WT + PTL | | Desmin KO | | KO + PTL | | Test |
| N (cells) | 22 | | 23 | | 23 | | 20 | | |
| E₁ (Pa) | 738.0 | (82.4) | 533.11 | (66.3) | 376.52 | (36.2) | 429.76 | (40.6) | |
| P (vs | | | 0.077 | | | | 0.926 | | ANOVA, |
| without | | | | | | | | | Tukey |
| PTL) | | | | | | | | | |
| E₂ (Pa) | 1008.9 | (119.1) | 627.4 | (89.3) | 486.9 | (90.7) | 512.2 | (79.5) | |
| P (vs | | | 0.029 | | | | 0.997 | | ANOVA, |
| without | | | | | | | | | Tukey |
| PTL) | | | | | | | | | |
| η (Pa*s) | 109.4 | (11.9) | 32.9 | (4.5) | 52.7 | (10.9) | 27.4 | (6.9) | |

| | | | | | |
|---|---|---|---|---|---|
| P (vs | | 1.96 × 10⁻⁷ | | 0.20 | ANOVA, |
| without | | | | | Tukey |
| PTL) | | | | | |
| SLSM Fit r² | 0.979 | 0.975 | 0.958 | 0.962 | |

### Microtubule Detyrosination is Sufficient to Impair Cardiomyocyte Contractility

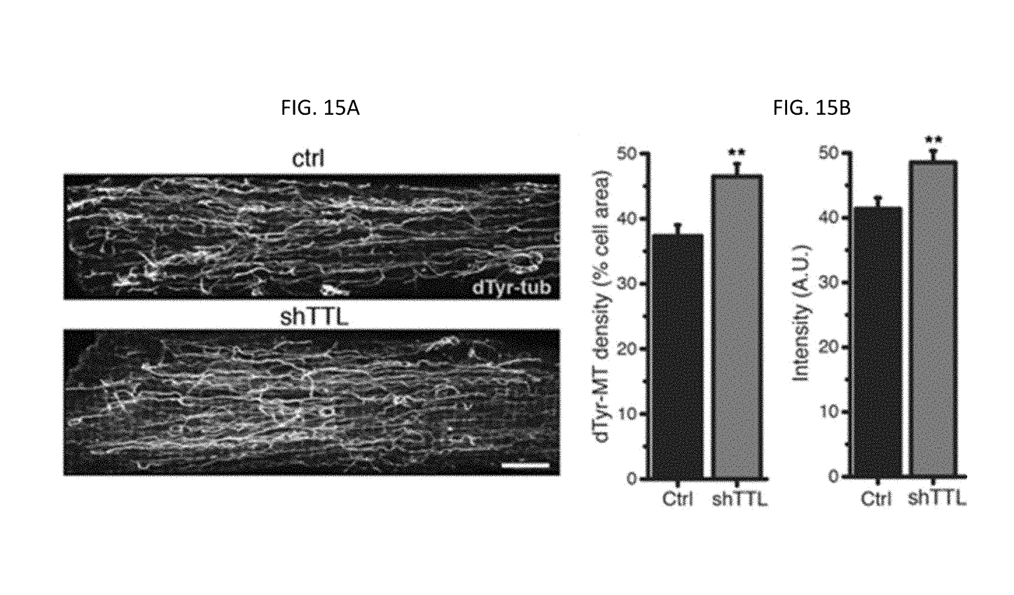

Increasing detyrosination correlates with impaired function in animal models of heart disease (14, 15). We thus next tested whether increasing detyrosination could directly impair cardiac contractility. Using an adenoviral construct expressing shRNAs against TTL (shTTL), we suppressed TTL expression, enhancing detyrosination (FIG. 7A, FIG. 15A-FIG. 15B). shTTL-transduced myocytes were then tested for their viscoelastic and contractile properties. The excess detyrosination alone was sufficient to increase viscosity and stiffness (FIG. 7B-FIG. 7C), suppressing contractile velocity and magnitude (FIG. 7D -FIG. 7E).

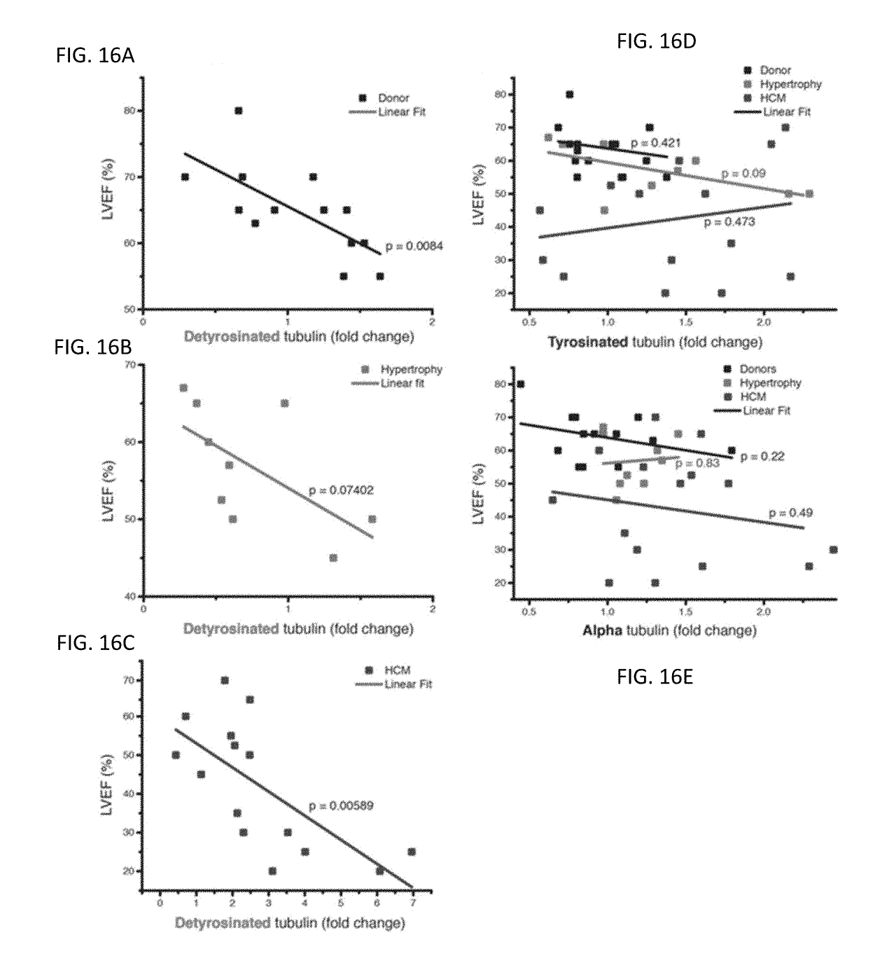

We next examined whether this modification correlated with functional deficits in human heart disease. To this end we analyzed left ventricular tissue samples from healthy patient donors and from patients exhibiting varying degrees of heart disease due to several underlying causes (Table 7). Detyrosinated tubulin was significantly increased in patients with clinically diagnosed hypertrophic and dilated cardiomyopathies (HCM and DCM, respectively), along with a modest increase in total tubulin content (FIG. 7F-FIG. 7G). Blind analysis of HCM patient data showed that detyrosination inversely correlated with left ventricular ejection fraction (LVEF), a primary indicator of cardiac contractility (FIG. 7H). There was no such correlation detected between LVEF and total or tyrosinated tubulin levels, nor any correlation between heart weight and detyrosination (FIG. 16A-FIG. 16E), suggesting a specific link between detyrosination and LVEF. Myocardium from patients with DCM all demonstrated significantly depressed LVEF and variable, but increased, detyrosinated tubulin.

TTL was unchanged in all patient populations, showing that a decreased expression of the tyrosinating enzyme does not explain the increase in detyrosinated tubulin in patients with heart disease (FIG. 7G). Because the molecular identity of TCP is unknown, it is unclear if upregulation of the detyrosinating enzyme may underlie this effect.

TABLE 7
Descriptive statistics of human patients.

| | No. Pa- | | Heart | Body Mass | |
|---|---|---|---|---|---|
| Group | tients | Age | Weight (g) | Index | LVEF (%) |
| Healthy | 17 | 49 ± 3.3 | 370 ± 18.7 | 27.8 ± 1.4 | 64.9 ± 1.5 |
| Donors | | | | | |
| Hyper- | 9 | 50.8 ± 2.6 | 513.2 ± 24.5 | 27.7 ± 1.6 | 57.2 ± 2.7 |
| trophy | | | | | |
| Donors | | | | | |
| DCM | 17 | 57.8 ± 2.0 | 566.9 ± 25.5 | 28.3 ± 2.8 | 13.2 ± 0.8 |
| Ischemia | 10 | 58.9 ± 1.6 | 674.4 ± 31.6 | 26.7 ± 1.8 | 13.3 ± 1.1 |
| PostVAD | 10 | 47 ± 4.9 | 501.4 ± 32.7 | 27.0 ± 1.6 | 31.1 ± 2.8 |
| DCM | | | | | |
| HCM | 15 | 50.9 ± 3.0 | 473.5 ± 31.4 | 26.4 ± 1.1 | 42.0 ± 4.3 |

TABLE 8
Statically parameters for AFM data for shTTL
experiments presented in FIG. 7A-FIG. 7H.

| | | | |
|---|---|---|---|
| Viscoelastic | | | |
| Parameters | Scramble | shTTL | Test |

| | | | |
|---|---|---|---|
| N (cells) | 25 | 26 | 5 |

| | | | | |
|---|---|---|---|---|
| E₁ (Pa) | 338.8 | (36.1) | 522.7 | (36.8) |

| | | |
|---|---|---|
| P (vs null/ctrl) | 7.96 × 10⁻⁴ | T-test |

| | | | | |
|---|---|---|---|---|
| E₂ (Pa) | 348.7 | (38.2) | 506.3 | (31.3) |

| | | |
|---|---|---|
| P (vs null/ctrl) | 0.002 | T-test |

| | | | | |
|---|---|---|---|---|
| η (Pa*s) | 61.6 | (11.8) | 119.4 | (14.8) |

| | | | |
|---|---|---|---|
| P (vs null/ctrl) | | 0.0038 | T-test |
| SLSM Fit r² | 0.97 | 0.97 | 15 |

TABLE 9

| | | | |
|---|---|---|---|
| Contractility | Scramble | shTTL | Test |

| | | |
|---|---|---|
| N (cells) | 64 | 65 |

| | | | | |
|---|---|---|---|---|
| Resting SL (μm) | 1.84 | (0.01) | 1.84 | (0.01) |

| | | |
|---|---|---|
| P (vs null/dmso) | 0.6588 | T-test |

| | | | | |
|---|---|---|---|---|
| ΔSL (μm) | 0.267 | (.008) | 0.223 | (0.01) |

| | | |
|---|---|---|
| P (vs null/dmso) | 0.0001 | T-test |

| | | | | |
|---|---|---|---|---|
| Vmax (μm/s) | −6.08 | (0.26) | −4.96 | (0.28) |

| | | |
|---|---|---|
| P (vs null/dmso) | 0.001 | T-test |

| | | | | |
|---|---|---|---|---|
| Vmin (μm/s) | 5.06 | (0.18) | 4.07 | (0.21) |

| | | |
|---|---|---|
| P (vs null/dmso) | 0.0002 | T-test |

### Discussion

Our findings demonstrate a regulatory pathway for MT load bearing and myocyte mechanics through post-translational detyrosination of tubulin. Detyrosinated microtubules buckle under load in contracting cardiomyocytes, conferring mechanical resistance to contraction and regulating the viscoelastic properties of the myocyte.

The observation that microtubules normally buckle rather than break or slide strongly indicates that they bear load and store elastic bending energy during the cardiac contractile cycle. This has implications for microtubule-dependent mechano-signaling in muscle and other tissues (1, 33, 34), but also direct implications on contractility. Our model of myocyte contractility demonstrates how changing MT load bearing and force transfer with the sarcomeres can significantly alter contractile properties. Our experimental data show that such changes in MT load bearing can be achieved by posttranslational modifications of the MTs themselves, particularly detyrosination. The measured reductions in buckling, viscoelasticity, and the increase in contractile speed of PTL treated/ITL overexpressing myocytes can all be attributed to changing the way MTs interact with the sarcomere and impairing their ability to act as compression resistors. It is also possible that detyrosinated MTs anchored to one sarcomere form bundles with MTs anchored to adjacent sarcomeres. If so, disrupting bundling would also effectively uncouple MTs from force generating structures. Regardless of the mechanism, disrupting coupling to sarcomeres would reduce the incident force on the MT, and buckling occurrence would drop.

The striking periodicity of buckles in untreated myocytes lends further support to the idea of a sarcomeric anchor. The preferential association of desmin with detyrosinated tubulin and insensitivity of desmin KO animals to changes in detyrosination strongly implicates desmin as at least one component of a sarcomeric anchoring complex of detyrosinated MTs. Interestingly, myocytes lacking desmin have decreased viscoelasticity, despite a denser MT network, supporting the idea that MT network organization and cross-linking is a stronger determinant of myocyte mechanical properties than network density per se. Both the desmin and MT networks have elements perpendicular to their typical orientation, particularly near the sarcolemma, which may alter how those elements interact with the cytoskeleton and plasma membrane. However we believe that the preponderance of the contractile resistance that results from detyrosination is due to longitudinal MTs in an orthogonal grid with transverse desmin IFs due to the simple numerical majority of cytoskeletal elements in this configuration.

Despite the fact that detyrosinated MTs store energy during sarcomere contraction, providing compression resistance, little of this energy appears to return in the form of a restoring force that would quicken sarcomere extension. This implies that energy used to deform MTs undergoes substantial loss. Buckling of the MT exerts compressive force on the surrounding matrix and deforms the cytoplasm, which due to its intrinsic viscosity, can act as an energy sink during each cycle. This is reflected in the large viscous component of the MT contributions to myocyte mechanical properties observed at deformation rates consistent with contractile velocities both in this and previous work (35). However we do note a slight prolongation of the late phase of relaxation in TTL overexpressing myocytes, which may represent the loss of a MT contribution to restoring force. We consider it probable that the restoring force of other internal elastic elements such as titin are likely to play a more dominant role, at least in the initial return towards resting sarcomere length (28). Thus, an increase in detyrosination may increase myocyte stiffness and impair contraction by acting as an energy sink, without providing significant energetic return during relaxation.

Consistent with this, an increase in detyrosination was associated with clinical contractile dysfunction in human hearts. Our cellular studies demonstrate that acute reduction of detyrosination with genetic or pharmacologic approaches can boost contractility and reduce mechanical stiffness. Additionally, these approaches are able to induce large changes in detyrosination while only slightly altering the overall MT cytoskeleton, minimizing off target consequences. Thus, interfering with detyrosination may represent an attractive and novel therapeutic strategy for increasing contractility.

In conclusion, our data show that microtubules exhibit divergent mechanical behavior due to the differences in how they couple to the rest of the cardiac cytoskeleton. The tyrosinated portions of the network, moving readily with the myocyte during contraction, provide little contractile resistance. Conversely the detyrosinated portions of the MT network, forming complexes with desmin intermediate filaments, produce a crosslinked MT/IF network that confers robust resistance to contraction. This orthogonal MT/IF grid requires tightly periodic MT deformations to accommodate myocyte morphology changes during contraction. These deformations require a significant amount of energy to form, and dissipate a large fraction of that energy due to viscous interactions. This has significant implications for MT load-bearing across cell biology, as well as for the altered mechanical stiffness and mechano-signaling in cardiac disease.

### Example 2—Suppression of Detyrosinated Microtubules Improves Cardiomyocyte Function in Human Heart Failure

The functional effects of MT detyrosination in heart failure or in human hearts have not previously been studied. We utilized mass spectrometry and single-myocyte mechanical assays to characterize changes to the cardiomyocyte cytoskeleton and their functional consequences in human heart failure. There were two major goals of this study. First, to broadly characterize how the cardiac cytoskeleton is changed in human heart disease of various etiology and severity. Second, to directly test whether detyrosinated MTs regulate the mechanics of human cardiomyocytes from non-failing and failing hearts. Together, our results support the attractiveness of detyrosinated MTs as a target for therapeutic intervention.

### Methods

Human Myocardial Tissue Procurement of human myocardial tissue was performed under protocols approved by Institutional Review Boards at the University of Pennsylvania and the Gift-of-Life Donor Program (Pennsylvania, USA). Failing human hearts were procured at the time of orthotropic heart transplantation at the Hospital of University of Pennsylvania. Non-failing (NF) hearts were obtained at the time of organ donation from cadaveric donors. In all cases, hearts were arrested in situ using ice-cold cardioplegia solution, transported on wet ice. Whole hearts and dissected left ventricle (LV) cavity were weighed to determine levels of hypertrophy. Transmural myocardial samples were dissected from the mid LV free wall below the papillary muscle. LV tissues for mass spectrometry and western blot were flash frozen in liquid nitrogen within 4 hours of explantation. Contractile parameters, including left ventricle ejection fraction (LVEF), were determined by echocardiography in patients.

Classification standard: NF donor hearts (with a LVEF greater than 50%) are further divided into normal and compensated hypertrophy (cHyp). cHyp is defined by an indexed LV mass (LV mass/body surface area) above 115 g/m² in men and 95 g/m² in women (44). Failing hearts are etiologically defined by clinical diagnosis of hypertrophic cardiomyopathy (HCM), which is subdivided into HCM with preserved EF (HCMpEF EF>50%) and HCM with reduced EF (HCMrEF EF<50%). Failing hearts with dilated LV chamber size are classified as dilated cardiomyopathy (DCM), and failing hearts with ischemic injury are grouped as ischemic cardiomyopathy (ICM). A Proportion of the failing hearts manifest a combination of mixed ischemic dilated etiology.

34 hearts in total were used in mass spectrometry (see additional method details below), including 7 normal, 6 cHyp, 4 HCMpEF, 5 HCMrEF, 6 DCM, 6 ICM. Myocytes were isolated from 22 hearts (see details below) for functional studies, including 6 normal and 7 cHyp, 9 failing transplants (all hearts with EF<50%, including 6 DCM, 1HCMpEF, 1 HCMrEF, 1 ICM). All data collected from these patients are included in this study.

### Human Left Ventricular Myocyte Isolation

Hearts received cold, blood-containing, high-potassium cardioplegic solution in vivo. Explanted hearts were transported from the operating suite to the laboratory in cold Krebs-Henseleit buffer (KHB) solution (12.5 mM glucose, 5.4 mM KCl, 1 mM lactic acid, 1.2 mM MgSO₄, 130 mM NaCl, 1.2 mM NaH₂PO4, 25 mM NaHCO₃, and 2 mM Na pyruvate, pH 7.4). Myocytes were disaggregated by use of a modification of isolation techniques described previously (45). Briefly, hearts were weighed and rinsed in KHB. A non-infarcted free wall region of the LV apex was dissected and a small catheter was placed into the lumen of left ventricular descending (LAD) artery. Major large vessels on the tissue piece were identified by injecting KHB via the cannula and tied by suture knots to improve perfusion via small vessels. Once the tissue was ready for perfusion, it was covered by plastic wrap with pores for outflow, in order to maintain tissue temperature at 37° C. The cannulated LV tissue was perfused with a non-recirculating Ca²⁺-free solution (KHB containing 20 mM BDM and 10 mM taurine) for 10-15 minutes until the outflow temperature reached around 37° C. Then, 200 mL of KHB containing 294 U/mL collagenase, 20 mM BDM and 10 mM taurine was perfused for 3 mins without recirculation and followed by 22-32 mins with recirculation (depending on the cannulation and how fibrotic the tissue was). Ca²′ was introduced stepwise per minute by adding CaCl₂ solution up to 1 mM, i.e. 4×50 μM, 4×100 μM and 2×200 μM into the recirculated collagenase solution. Then the tissue was perfused for 5 minutes with rinse solution (KHB containing 10 mM taurine, 20 mM BDM, 1 mM CaCl₂) and 1% BSA). The tissue was then removed from the cannula, and myocardium tissue was minced in the rinse solution and triturated using glass pipets. The resulting cell suspension was filtered through 280 μm nylon mesh (Component supply U-CMN-280), centrifuged (25 g for 2 min), and resuspended in rinse solution. The temperature was maintained at 37° C. throughout the isolation. Viable cells were enriched by gravity-sedimentation for 5 min, and resulted loose pellet was transferred to a fresh tube and resuspended in proper amount of normal Tyrode's (NT) solution for contractility, [Ca²⁺]ᵢ transients and nanoindentation. Cells were also fixed for immunofluorescence (see details below).

### Mass Spectrometry of Human Left Ventricular Tissues

Sample preparation: Human left ventricular tissues collected from a relatively healthy (myocardial rich, minimally fibrotic) region of the mid LV wall were used for mass spectrometry. Tissue was homogenized in ice-cold RIPA lysis buffer containing 0.1% SDS (Cayman 10010263) supplemented with protease inhibitor cocktail (Sigma). Protein concentration was determined by protein assay dye reagent (Bio-Rad 5000205). 100 μg protein from each sample was precipitated as previously described (46). The pellet was resuspended with 8 M urea, 100 mM NH4HCO3, and pH 8.0. Denaturation/reduction was performed in 8 M urea/5 mM dithiothreitol/25 mM NH₄HCO₃ (pH 8.0) for over 60 min in 52° C. The solution was stored at room temperature in 25 mM iodoacetamide at the dark for 60 min. The urea was diluted to a concentration of 1 M with 25 mM NH₄HCO₃ and then digested with trypsin (1:50 ratio) at 37° C. with shaking for 16 hrs. After tryptic digestion, peptide mixture was desalted with C18 micro spin column (C18, Harvard Apparatus, and Holliston, Mass.). The column was washed with 200 μL of 100% acetonitrile and equilibrated with 200 μL of loading buffer (0.1% formic acid). Peptides were loaded onto the column, washed with a loading buffer and eluted with 200 μL of 70% acetonitrile/0.1% formic acid. All steps for loading, washing, and elution were carried out with benchtop centrifugation (300×g for 2 min). The eluted samples were dried in a centrifugal vacuum concentrator, reconstituted with 0.1% formic acid.

Nano Liquid Chromatography and Mass Spectrometry:

Desalted peptides were analyzed on a Q-Exactive (Thermo Scientific) attached to an EasyLC system run at 300 nL/min. Peptides were eluted with a 170 min gradient from 2% to 32% ACN and to 98% ACN over 10 min in 0.1% formic acid. Data dependent acquisition mode with a dynamic exclusion of 45 s was enabled. One full MS scan was collected with scan range of 350 to 1600 m/z, resolution of 70 K, maximum injection time of 50 ms and automatic gain control (AGC) of 1E6. Then, a series of MS2 scans were acquired for the most abundant ions from the MS1 scan (top 12). Ions were filtered with charge 2-4. An isolation window of 2.0 m/z was used with quadruple isolation mode. Ions were fragmented using higher-energy collisional dissociation (HCD) with collision energy of 27%. Orbitrap detection was used with scan range of 200 to 2000 m/z, resolution of 17.5 K, maximum injection time of 150 ms and automatic gain control of 5E6.

Proteomics MS Data Analysis Including Peptide Identification and Quantification:

MaxQuant version 1.5.3.30 was used to process the raw spectra (47). The uniprot human database was used for database searching. Default search parameters were used, including precursor mass tolerance of 20 ppm, fragment mass tolerance of 20 ppm, trypsin cleavage and up to 2 mis-cleavage. Carbamidomethyl [C] was set as fixed modification, while Oxidation [M] was set as variable modifications. The target-decoy approach was used to filter the search results (48), in which the false discovery rate was less than 1% at the peptide and protein level. LFQ (Label-free quantification) and iBAQ (intensity-based absolute-protein-quantification) were enabled.

### Proteomic Analysis

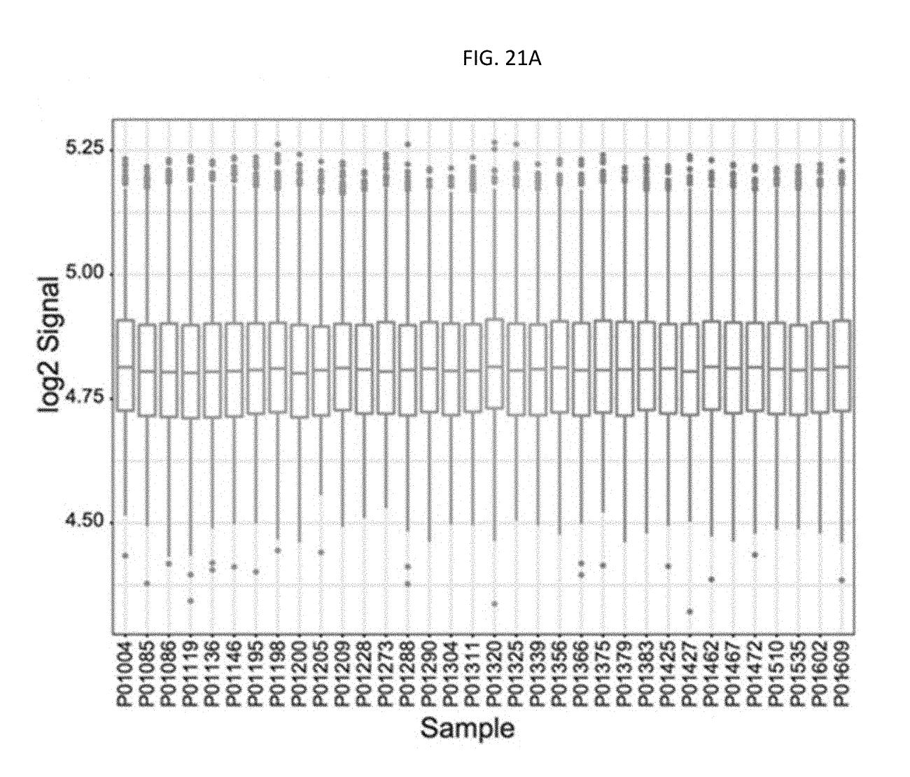

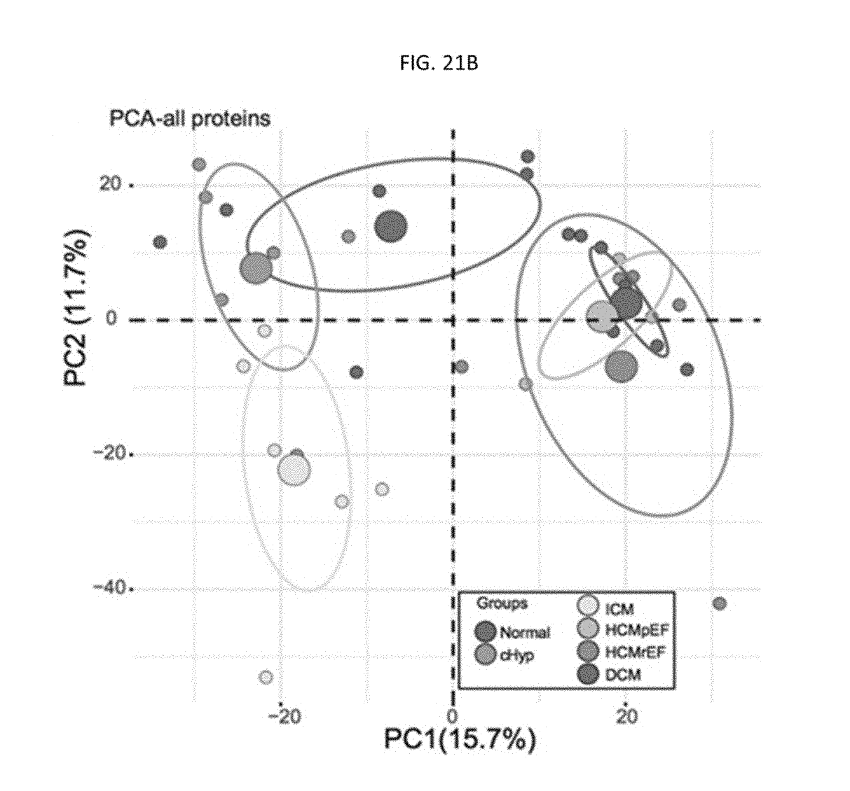

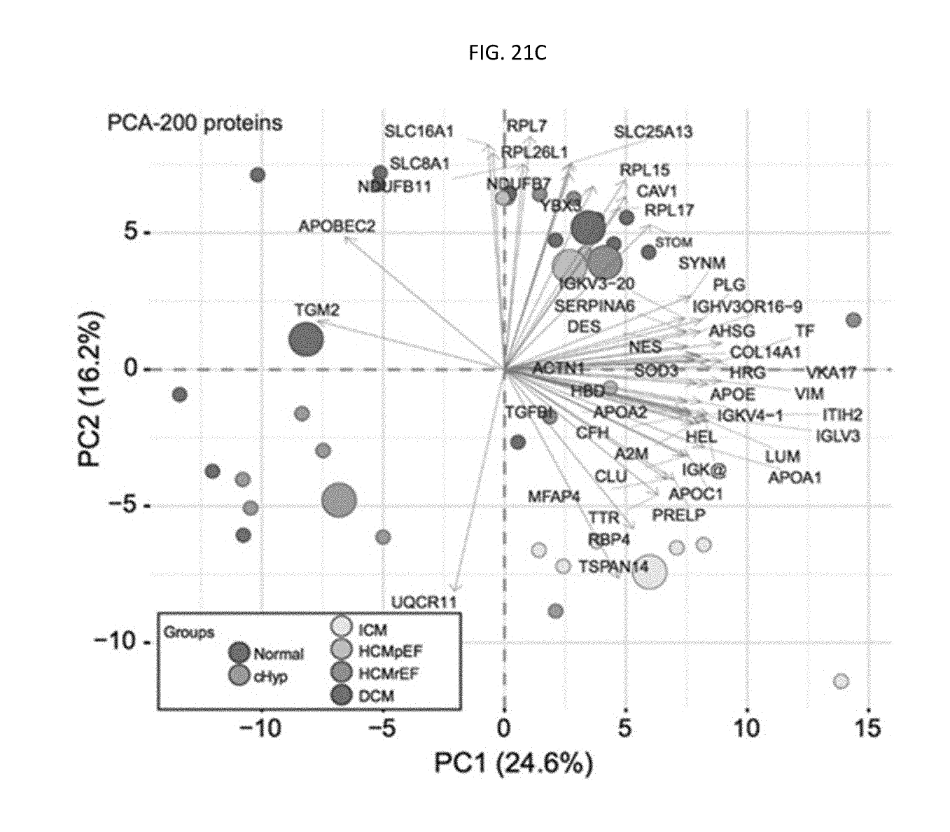

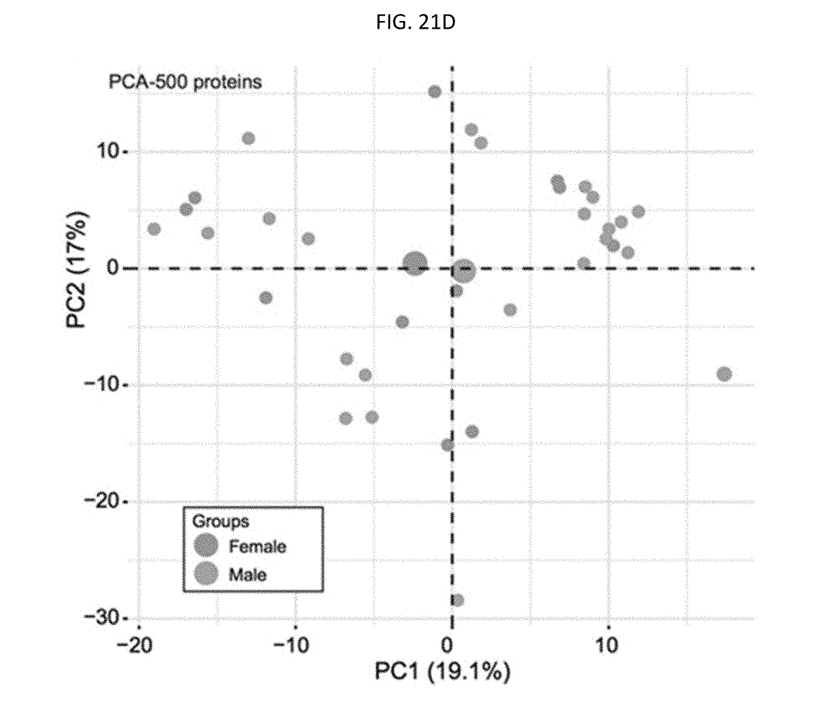

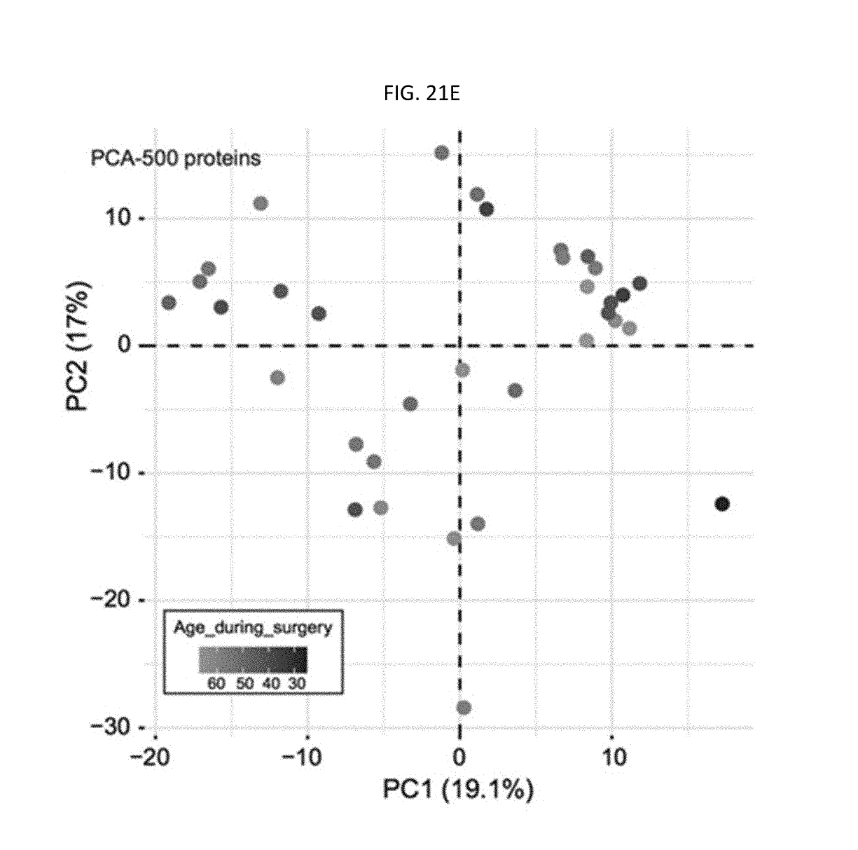

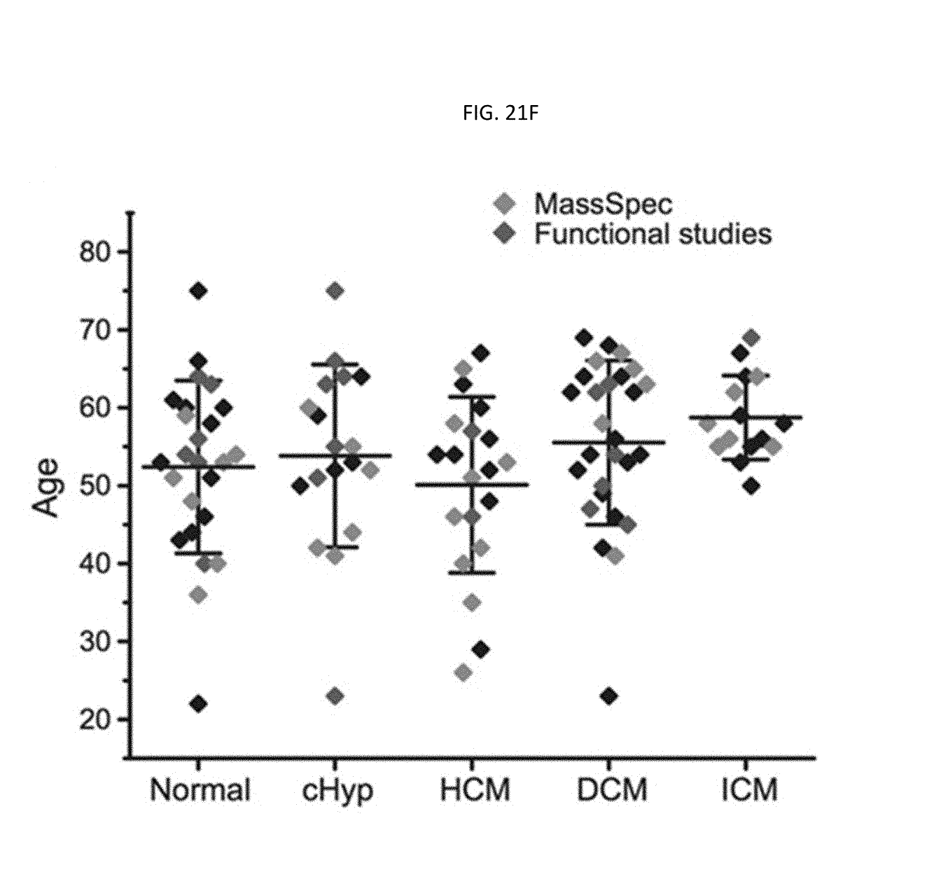

LFQ values measured in mass spectrometry were used to represent the expression levels of proteins. Box plots of sample variance indicate normal distributions in each sample (FIG. 21A). Duplicated protein entries with lower LFQ values were removed. Also, protein entries with a median LFQ value of 0 were removed to exclude proteins expressed at very low levels or undetected by mass spectrometry. This resulted in a list of 2,676 proteins. To assess prominent changes in different disease groups, the protein list was ranked by statistical significance obtained in differential gene expression analysis. A linear model adjusting for age and sex in the R package Limma was used. P values were adjusted for multiple testing using the Benjamini-Hochberg procedure. Table 10 is a summary of top 25 up- and down-regulated proteins in differential gene expression analysis.

TABLE 10

| | | | |
|---|---|---|---|
| Gene | | | fold |
| symbol | Gene name | adj. P. Val | change |

up-regulated
cHyp vs. normal

| | | | |
|---|---|---|---|
| FHL1 | Four and a half LIM domains protein 1 | 0.0004 | 2.075 |
| HSPB6 | Heat shock protein beta-6 | 0.0047 | 2.302 |
| HSPA2 | Heat shock-related 70 kDa protein 2 | 0.0105 | 5.349 |
| COL18A1 | Collagenalpha-1(XVIII)chain; Endostatin | 0.0150 | 2.978 |
| RBP4 | Retinol-binding protein4 | 0.0253 | 3.501 |
| COL1A1 | Collagen Type I Alpha 1 Chain | 0.0253 | 3.400 |
| VCL | Vinculin | 0.0661 | 1.608 |
| UQCR11 | Ubiquinol-Cytochrome C Reductase, | 0.0688 | 2.631 |
| | Complex III Subunit XI | | |
| GLUD2 | Glutamate dehydrogenase 2, mitochondrial | 0.0688 | 1.386 |
| HK1 | Hexokinase-1 | 0.0688 | 1.430 |
| CRK | Adapter molecule crk | 0.0688 | 1.920 |
| ACTA1 | Actin, alpha skeletal muscle | 0.0688 | 3.889 |
| DDAH1 | N(G),N(G)-dimethyl arginine dimethyl | 0.0688 | 2.117 |
| | amino hydrolase1 | | |
| TOM1L2 | TOM1-like protein2 | 0.0688 | 1.324 |
| PDXP | Pyridoxal phosphate phosphatase | 0.0754 | 1.617 |
| CPOX | Oxygen-dependent coproporphyrinogen-III | 0.0758 | 1.797 |
| | oxidase, mitochondrial | | |
| PYGB | Glycogen phosphorylase, brainform | 0.0815 | 1.458 |
| MAP4 | Microtubule-associated protein 4 | 0.0837 | 1.281 |
| BPNT1 | 3(2),5-bisphosphate nucleotidase 1 | 0.0877 | 1.553 |
| ALDH9A1 | 4-Trimethylaminobutyraldehyde | 0.0877 | 1.331 |
| | Dehydrogenase | | |
| RPL11 | 60S ribosomal protein L11 | 0.0877 | 1.547 |
| GNPDA1 | Glucosamine-6-phosphate isomerase 1 | 0.0877 | 1.355 |
| RRAS2 | Ras-related protein R-Ras2 | 0.0923 | 1.727 |
| CFL2 | Cofilin-2 | 0.0954 | 1.416 |
| PGAM1 | Phosphoglycerate mutase 1 | 0.1106 | 1.352 |

ICM vs. normal

| | | | |
|---|---|---|---|
| RBP4 | Retinol-bindingprotein4 | 0.0001 | 6.595 |
| TF | Transferrin | 0.0001 | 2.945 |
| APOA4 | Apolipoprotein A-IV | 0.0001 | 15.391 |
| DES | Desmin | 0.0004 | 6.608 |
| SERPINA6 | Corticosteroid-binding globulin | 0.0004 | 2.667 |
| AHSG | Alpha-2-HS-glycoprotein | 0.0004 | 2.825 |
| VIM | Vimentin | 0.0004 | 4.753 |
| TSPAN14 | Tetraspanin-14 | 0.0004 | 4.413 |
| TTR | Transthyretin | 0.0004 | 14.009 |
| FHL1 | Four and a half LIM domains protein 1 | 0.0004 | 1.872 |
| CLU | Clusterin | 0.0004 | 4.202 |
| HSPB6 | Heat shock protein beta-6 | 0.0004 | 2.352 |
| DDAH1 | N(G),N(G)-dimethyl arginine dimethyl | 0.0004 | 3.098 |
| | aminohydrolase 1 | | |
| COL18A1 | Collagenalpha-1(XVIII) chain | 0.0005 | 3.507 |
| KNG1 | Kininogen-1 | 0.0005 | 2.775 |
| APOA1 | Apolipoprotein A-I | 0.0005 | 3.109 |
| MFAP4 | Microfibril-associated glycoprotein 4 | 0.0006 | 4.989 |
| CLEC3B | Tetranectin | 0.0006 | 2.692 |
| PRELP | Prolargin | 0.0006 | 4.532 |
| MAP4 | Microtubule-associated protein 4 | 0.0006 | 1.444 |
| HSPA2 | Heatshock-related 70 kDa protein 2 | 0.0008 | 5.728 |
| LUM | Lumican | 0.0008 | 2.880 |
| NES | Nestin | 0.0008 | 3.343 |
| TGFBI | Transforming growth factor-beta-induced | 0.0009 | 3.906 |
| | protein Ig-h3 | | |
| ITIH2 | Inter-alpha-trypsin inhibitor heavy chain H2 | 0.0009 | 3.038 |

HCMpEF vs. normal

| | | | |
|---|---|---|---|
| THBS4 | Thrombospondin-4 | 0.0049 | 10.662 |
| PGM5 | Phosphoglucomutase-like protein5 | 0.0063 | 1.844 |
| SERPINA6 | Corticosteroid-binding globulin | 0.0065 | 2.525 |
| TF | Transferrin | 0.0065 | 2.478 |
| AHSG | Alpha-2-HS-glycoprotein | 0.0102 | 2.505 |
| NES | Nestin | 0.0102 | 3.316 |
| SGCE | Epsilon-sarcoglycan | 0.0191 | 2.270 |
| V<kappa>3 | Rheumatoid factor D5 light chain | 0.0211 | 4.479 |
| CA3 | Carbonicanhydrase3 | 0.0236 | 12.702 |
| APOA4 | ApolipoproteinA-IV | 0.0236 | 7.011 |
| SYNM | Synemin | 0.0236 | 2.329 |
| MXRA7 | Matrix-remodeling-associated protein 7 | 0.0259 | 2.003 |
| TGFBI | Transforming growth factor-beta-induced | 0.0263 | 3.295 |
| | protein Ig-h3 | | |
| DDAH1 | N(G),N(G)-dimethyl arginine dimethyl | 0.0289 | 2.643 |
| | aminohydrolase1 | | |
| SLMAP | Sarcolemmal membrane-associated protein | 0.0314 | 1.650 |
| PTGFRN | Prostaglandin F2 Receptor Inhibitor | 0.0323 | 2.146 |
| HMGN2 | Non-histone chromosomal protein HMG-17 | 0.0334 | 3.112 |
| TUBA1A | Tubulin alpha-1A chain | 0.0325 | 1.784 |
| F12 | Coagulation Factor XII | 0.0410 | 2.453 |
| ACTN1 | Alpha-actinin-1 | 0.0410 | 2.602 |
| PSAP | Prosaposin | 0.0410 | 1.641 |
| SLC44A2 | Solute Carrier Family 44 Member 2 | 0.0418 | 1.995 |
| SOD2 | Superoxide dismutase | 0.0418 | 1.735 |
| HRG | Histidine-rich glycoprotein | 0.0418 | 4.808 |
| HNRNPD | Heterogeneous nuclear ribonucleoprotein D0 | 0.0418 | 1.434 |

HCMrEF vs. normal

| | | | |
|---|---|---|---|
| THBS4 | Thrombospondin-4 | 0.0001 | 16.577 |
| HSPA2 | Heat shock-related 70 kDa protein2 | 0.0002 | 9.007 |
| NES | Nestin | 0.0005 | 4.268 |
| DDAH1 | N(G),N(G)-dimethyl arginine dimethyl | 0.0007 | 3.271 |
| | aminohydrolase1 | | |
| AHSG | Alpha-2-HS-glycoprotein | 0.0009 | 2.796 |
| MAP4 | Microtubule-associated protein 4 | 0.0009 | 1.487 |
| DES | Desmin | 0.0012 | 5.832 |
| MXRA7 | Matrix-remodeling-associated protein 7 | 0.0014 | 2.320 |
| ARHGAP1 | RhoGTPase-activating protein 1 | 0.0014 | 2.023 |
| SLMAP | Sarcolemmal membrane-associated protein | 0.0016 | 1.862 |
| TF | Transferrin | 0.0022 | 2.383 |
| COL14A1 | Collagenalpha-1(XIV)chain | 0.0027 | 24.444 |
| SVIL | Supervillin | 0.0027 | 3.256 |
| SYNM | Synemin | 0.0028 | 2.542 |
| MFGE8 | Lactadherin | 0.0028 | 2.859 |
| PTGFRN | Prostaglandin F2 Receptor Inhibitor | 0.0041 | 2.369 |
| COMT | Catechol-O-methyltransferase | 0.0045 | 1.461 |
| AEBP1 | Adipocyte enhancer-binding protein 1 | 0.0045 | 5.828 |
| PGM5 | Phospho glucomutase-like protein 5 | 0.0045 | 1.668 |
| CLEC3B | Tetranectin | 0.0049 | 2.394 |
| PPP1R12C | Protein phosphatase 1regulatory | 0.0056 | 2.552 |
| | subunit 12C | | |
| APOA4 | Apolipoprotein A-IV | 0.0056 | 7.306 |
| SLC44A2 | Solute Carrier Family 44 Member 2 | 0.0056 | 2.192 |
| TUBA1A | Tubulin alpha-1A chain | 0.0058 | 1.871 |
| ALDH3A2 | Fatty aldehyde dehydrogenase | 0.0061 | 2.167 |

DCM vs. normal

| | | | |
|---|---|---|---|
| SVIL | Supervillin | 0.0003 | 4.262 |
| DES | Desmin | 0.0003 | 7.144 |
| DDAH1 | N(G),N(G)-dimethyl arginine dimethyl | 0.0006 | 3.217 |
| | aminohydrolase1 | | |
| PGM5 | Phospho glucomutase-like protein5 | 0.0007 | 1.823 |
| SYNM | Synemin | 0.0009 | 2.762 |
| RTN3 | Reticulon-3 | 0.0009 | 2.773 |
| SLMAP | Sarcolemmal membrane-associated protein | 0.0009 | 1.867 |
| MXRA7 | Matrix-remodeling-associated protein7 | 0.0011 | 2.271 |
| APOA4 | Apolipoprotein A-IV | 0.0013 | 9.009 |
| PPP1R12C | Protein phosphatase 1 regulatory subunit 12C | 0.0013 | 2.811 |
| AHSG | Alpha-2-HS-glycoprotein | 0.0014 | 2.497 |
| SPTBN1 | Spectrin beta chain, non-erythrocytic 1 | 0.0014 | 1.663 |
| NES | Nestin | 0.0014 | 3.267 |
| VIM | Vimentin | 0.0015 | 3.924 |
| TTN | Titin | 0.0015 | 3.859 |
| HP1BP3 | Hetero chromatin proteinl-binding protein 3 | 0.0015 | 1.699 |
| PTGFRN | Prostaglandin F2 Receptor Inhibitor | 0.0016 | 2.409 |
| SGCE | Epsilon-sarcoglycan | 0.0017 | 2.325 |
| V<kappa>3 | Rheumatoid factor D5 light chain | 0.0019 | 5.203 |
| MAP4 | Microtubule-associated protein 4 | 0.0022 | 1.393 |
| TOMM40L | Mitochondrial import receptor subunit | 0.0025 | 5.121 |
| | TOM 40B | | |
| YWHAQ | 14-3-3 protein theta | 0.0027 | 1.665 |
| ARHGAP1 | RhoGTPase-activating protein1 | 0.0029 | 1.824 |
| TF | Transferrin | 0.0029 | 2.165 |
| THBS4 | Thrombospondin-4 | 0.0029 | 6.406 |

down-regulated
cHyp vs. normal

| | | | |
|---|---|---|---|
| COX17 | Cytochrome c oxidase copper chaperone | 0.0047 | −2.354 |
| CHCHD7 | Coiled-coil-helix-coiled-coil-helix | 0.0253 | −2.015 |
| | domain-containing protein 7 | | |
| MRPS35 | 28S ribosomal protein S35, mitochondrial | 0.0346 | −1.951 |
| ARMT1 | Protein-glutamate O-methyl transferase | 0.0350 | −1.844 |
| RPL27A | 60S ribosomal protein L 27a | 0.0688 | −3.845 |
| PLIN4 | Perilipin-4 | 0.0688 | −1.667 |
| COX3 | Cytochrome c oxidase subunit 3 | 0.0688 | −5.432 |
| MARCKS | Myristoylated alanine-rich C-kinase | 0.0688 | −1.772 |
| | substrate | | |
| CHORDC1 | Cysteine and histidine-rich domain- | 0.0688 | −2.785 |
| | containing protein 1 | | |
| DNAJC11 | DnaJ homolog subfamily C member 11 | 0.0688 | −3.052 |
| ENG | Endoglin | 0.0815 | −2.592 |
| ATP2A2 | Sarcoplasmic/endoplasmic reticulum | 0.0877 | −1.526 |
| | calcium ATPase 2 | | |
| RPL4 | 60S ribosomal protein L4 | 0.0877 | −1.580 |
| RAB12 | Ras-related protein Rab-12 | 0.0923 | −1.804 |
| BCAM | Basal cell adhesion molecule | 0.1060 | −1.528 |
| LYRM7 | Complex III assembly factor LYRM7 | 0.1276 | −2.326 |
| TPPP | Tubulin polymerization-promoting protein | 0.1276 | −1.754 |
| NHP2L1 | NHP2-likeprotein1, N-terminally processed | 0.1304 | −1.897 |
| RPL21 | 608 ribosomal protein L21 | 0.1276 | −2.765 |
| MAP2K4 | Dual specificity mitogen-activated protein | 0.1304 | −1.808 |
| | kinase kinase4 | | |
| CRIP2 | Cysteine-rich protein 2 | 0.1304 | −1.393 |
| CHCHD4 | Mitochondrial intermembrane space import | 0.1304 | −1.828 |
| | and assembly protein 40 | | |
| ACAD9 | Acyl-CoA dehydrogenase family member 9, | 0.1304 | −1.359 |
| | mitochondrial | | |
| CD34 | Hematopoietic progenitor cell antigen CD34 | 0.1304 | −2.010 |
| RPL6 | 60S ribosomal protein L6 | 0.1304 | −1.587 |

ICM vs. normal

| | | | |
|---|---|---|---|
| SAA1 | Serum amyloid A-1 protein | 0.0001 | −96.496 |
| ATP1A1 | Sodium/potassium-transporting ATPase | 0.0001 | −1.988 |
| | subunit alpha-1 | | |
| COX17 | Cytochrome c oxidase copper chaperone | 0.0004 | −2.578 |
| GPT | Alanine amino transferase 1 | 0.0004 | −2.276 |
| BCAM | Basal cell adhesion molecule | 0.0004 | −2.049 |
| LGALS1 | Galectin-1 | 0.0004 | −3.018 |
| BCL2L13 | Bcl-2-like protein 13 | 0.0006 | −1.731 |
| UQCC1 | Ubiquinol-cytochrome-c reductase | 0.0008 | −1.855 |
| | complex assembly factor 1 | | |
| ATP2A2 | Sarcoplasmic/endoplasmic reticulum | 0.0011 | −1.831 |
| | calcium ATPase 2 | | |
| PFKM | ATP-dependent 6-phosphofructokinase, | 0.0013 | −1.725 |
| | muscle type | | |
| QARS | Glutamine--tRNA ligase | 0.0015 | −2.707 |
| GALK1 | Galactokinase | 0.0015 | −2.127 |
| ATP1A3 | Sodium/potassium-transporting ATPase | 0.0017 | −1.698 |
| | subunit alpha-3 | | |
| MYH11 | Myosin-11 | 0.0017 | −18.805 |
| STAT3 | Signal transducer and activator of | 0.0019 | −2.151 |
| | transcription 3 | | |
| MRPS35 | 28S ribosomal protein S35, mitochondrial | 0.0023 | −2.250 |
| PYGM | Glycogen phosphorylase, muscle form | 0.0023 | −1.691 |
| SRPRB | Signal recognition particle receptor | 0.0026 | −2.870 |
| | subunit beta | | |
| ACAD9 | Acyl-CoA dehydrogenase family member 9, | 0.0035 | −1.536 |
| | mitochondrial | | |
| PERM1 | PGC-1 and ERR-induced regulator in | 0.0040 | −3.203 |
| | muscle protein 1 | | |
| SLC9A3R2 | Na(+)/H(+) exchange regulatory | 0.0040 | −1.966 |
| | cofactor NHE-RF2 | | |
| RPL7 | 60S ribosomal protein L7 | 0.0042 | −2.270 |
| ATP1B1 | Sodium/potassium-transporting ATPase | 0.0042 | −1.832 |
| | subunit beta-1 | | |
| APOBEC2 | Probable C−>U-editing enzyme | 0.0042 | −5.191 |
| | APOBEC-2 | | |
| SLC16A1 | Monocarboxylate transporter 1 | 0.0042 | −2.752 |

HCMpEF vs. normal

| | | | |
|---|---|---|---|
| LGALS1 | Galectin-1 | 0.0002 | −4.694 |
| SAA1 | Serum amyloid A-1 protein | 0.0027 | −76.665 |
| EIF5 | Eukaryotic translation initiation factor 5 | 0.0028 | −1.706 |
| GOT1 | Aspartate aminotransferase, cytoplasmic | 0.0065 | −1.610 |
| CD163 | Scavenger receptor cysteine-rich type1 | 0.0065 | −21.143 |
| | protein M130 | | |
| MYH6 | Myosin-6 | 0.0065 | −22.110 |
| CKM | Creatine kinase M-type | 0.0102 | −1.859 |
| NAMPT | Nicotinamide phosphoribosyl transferase | 0.0102 | −2.137 |
| ENO3 | Beta-enolase | 0.0102 | −2.142 |
| TPM2 | Tropomyosin beta chain | 0.0107 | −2.482 |
| PGAM2 | Phosphoglycerate mutase 2 | 0.0183 | −1.598 |
| PALLD | Palladin | 0.0211 | −1.785 |
| GPT | Alanine aminotransferase 1 | 0.0211 | −1.927 |
| DSTN | Destrin | 0.0211 | −1.472 |
| PYGM | Glycogen phosphorylase, muscle form | 0.0211 | −1.700 |
| SAR1A | GTP-binding protein SAR1a | 0.0236 | −2.193 |
| UBE2K | Ubiquitin-conjugating enzyme E2K | 0.0236 | −1.955 |
| ALDOA | Fructose-bisphosphate aldolase A | 0.0263 | −1.486 |
| MPI | Mannose-6-phosphate isomerase | 0.0265 | −2.053 |
| SERPINA3 | Alpha-1-antichymotrypsin | 0.0278 | −2.542 |
| HSPA1B | Heat shock 70 kDa protein 1B | 0.0278 | −1.531 |
| STK4 | Serine/threonine-protein kinase 4 | 0.0323 | −2.081 |
| FKBP4 | Peptidyl-prolylcis-trans isomerase | 0.0325 | −1.915 |
| | FKBP4, N-terminally processed | | |
| EIF1B | Eukaryotic translation initiation factor 1b | 0.0344 | −1.999 |
| CNDP2 | Cytosolic non-specific dipeptidase | 0.0410 | −1.315 |

HCMrEF vs. normal

| | | | |
|---|---|---|---|
| SAA1 | Serum amyloid A-1 protein | 0.0000 | −324.192 |
| LGALS1 | Galectin-1 | 0.0000 | −4.538 |
| NAMPT | Nicotinamide phosphoribosyl transferase | 0.0000 | −2.974 |
| SIAE | Sialate O-acetylesterase | 0.0006 | −2.456 |
| GOT1 | Aspartate aminotransferase, cytoplasmic | 0.0006 | −1.679 |
| CRP | C-reactive protein | 0.0007 | −31.556 |
| PGAM2 | Phosphoglycerate mutase2 | 0.0007 | −1.764 |
| GPT | Alanine aminotransferase1 | 0.0011 | −2.192 |
| MYH6 | Myosin-6 | 0.0020 | −19.536 |
| SERPINA3 | Alpha-1-antichymotrypsin | 0.0020 | −3.042 |
| LGALS3 | Galectin-3 | 0.0025 | −2.357 |
| CKM | Creatine kinase M-type, N-terminally | 0.0026 | −1.848 |
| | processed | | |
| PSME1 | Proteasome activator complex subunit 1 | 0.0027 | −1.423 |
| HIBADH | 3-hydroxy isobutyrate dehydrogenase, | 0.0027 | −1.755 |
| | mitochondrial | | |
| PDCD5 | Programmed cell death protein 5 | 0.0056 | −1.645 |
| HSPA1B | Heat shock70 kDa protein 1B | 0.0056 | −1.568 |
| ACSS3 | Acyl-CoA synthetase short-chain | 0.0066 | −2.640 |
| | family member3, mitochondrial | | |
| CD163 | Scavenger receptor cysteine-rich type1 | 0.0078 | −9.527 |
| | protein M130 | | |
| ACSF2 | Acyl-CoA synthetase family member2, | 0.0079 | −3.551 |
| | mitochondrial | | |
| PSME2 | Proteasome activator complex subunit 2 | 0.0087 | −1.611 |
| DECR1 | 2,4-dienoyl-CoA reductase, mitochondrial | 0.0089 | −1.708 |
| CUTA | Protein CutA | 0.0090 | −1.660 |
| HSP90AA1 | Heat shock protein HSP90-alpha | 0.0100 | −1.432 |
| AKR1B1 | Aldose reductase | 0.0102 | −1.586 |
| IDH2 | Isocitrate dehydrogenase[NADP], | 0.0102 | −1.773 |
| | mitochondrial | | |

DCM vs. normal

| | | | |
|---|---|---|---|
| SAA1 | Serum amyloid A-1 protein | 0.0000 | −255.359 |
| LGALS1 | Galectin-1 | 0.0000 | −4.611 |
| PGAM2 | Phosphoglycerate mutase2 | 0.0006 | −1.768 |
| MYH6 | Myosin-6 | 0.0006 | −30.569 |
| GALK1 | Galactokinase | 0.0007 | −2.582 |
| CD163 | Scavenger receptor cysteine-rich type 1 | 0.0009 | −17.824 |
| | protein M130 | | |
| SERPINA3 | Alpha-1-antichymotrypsin | 0.0011 | −3.087 |
| NAMPT | Nicotinamide phosphoribosyl transferase | 0.0013 | −2.163 |
| ANXA5 | AnnexinA5 | 0.0013 | −1.950 |
| CRP | C-reactive protein | 0.0014 | −32.149 |
| PSME1 | Proteasome activator complex subunit 1 | 0.0014 | −1.421 |
| SIAE | Sialate O-acetylesterase | 0.0014 | −2.114 |
| CKM | Creatine kinase M-type, N-terminally | 0.0017 | −1.805 |
| | processed | | |
| CSDC2 | Cold shock domain-containing protein C2 | 0.0018 | −2.463 |
| PDCD5 | Programmed cell death protein 5 | 0.0026 | −1.656 |
| VBP1 | Prefoldin subunit 3 | 0.0049 | −1.795 |
| CNDP2 | Cytosolic non-specific dipeptidase | 0.0050 | −1.336 |
| CUTA | Protein CutA | 0.0060 | −1.645 |
| PSME2 | Proteasome activator complex subunit2 | 0.0062 | −1.587 |
| ALDOA | Fructose-bisphosphate aldolase A | 0.0070 | −1.455 |
| ENO3 | Beta-enolase | 0.0070 | −1.873 |
| TPPP | Tubulin polymerization-promoting protein | 0.0076 | −2.053 |
| FAHD1 | Acylpyruvase FAHD1, mitochondrial | 0.0082 | −1.544 |
| DBI | Acyl-CoA-binding protein | 0.0090 | −1.704 |
| GPT | Alanine aminotransferase 1 | 0.0097 | −1.767 |

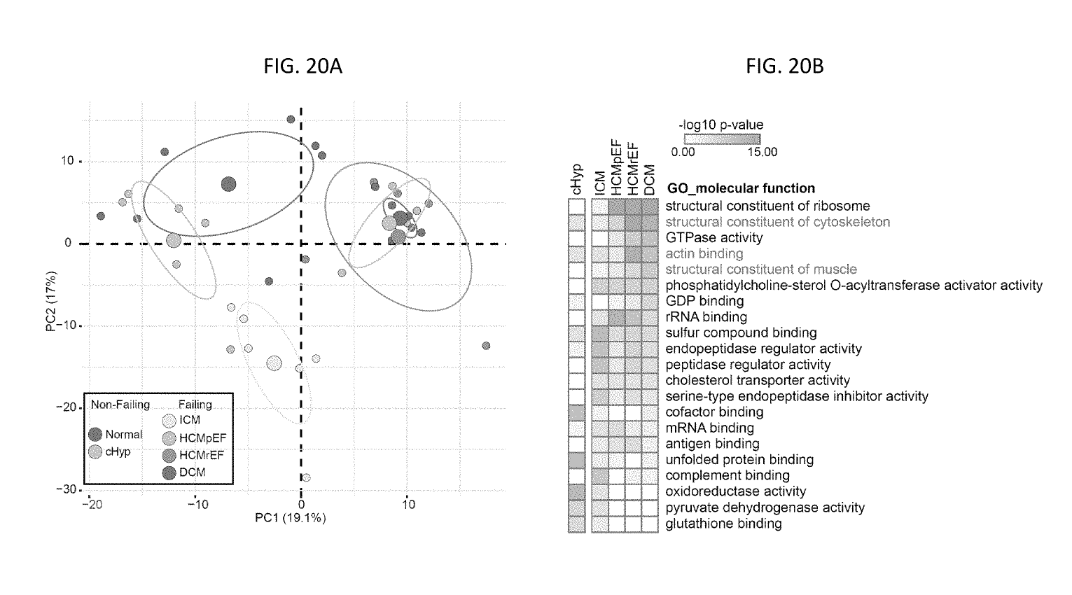

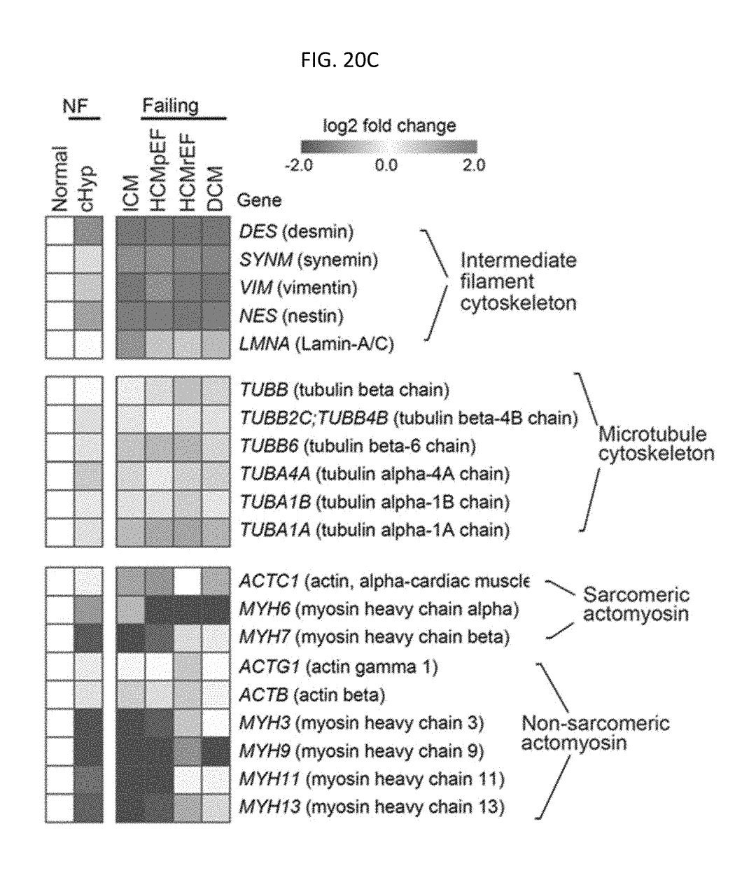

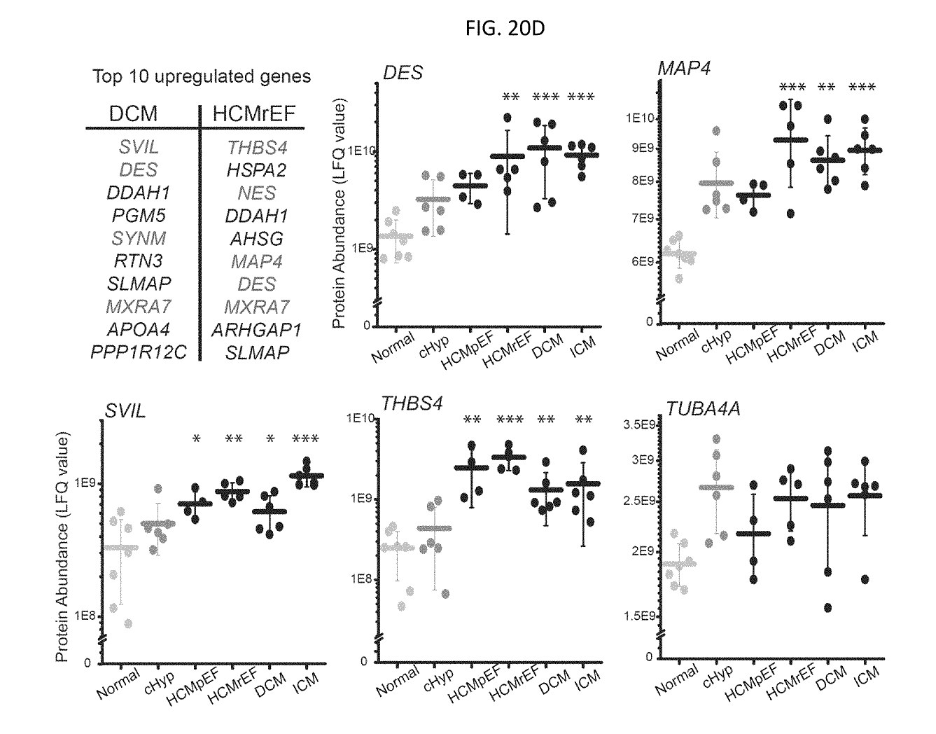

To obtain a general view of sample distribution among disease conditions and evaluate whether there is biased distribution toward certain gender or age groups, 500 (out of 2676) genes with highest variance among all samples were input in PCA performed using the FactoMineR R software package. Samples in the PCA plots are color coded by disease groups, age and gender to illustrate the distribution based on these factors (FIG. 20A, FIG. 21B-FIG. 21E). To concentrate on difference from the most variate proteins and discover the genes contributing to the difference, 2D-PCA biplot was generated (FIG. 21B).

To evaluate functional outcome beyond the level of individual proteins, gene list functional enrichment analysis (Toppfun) was performed on Toppgene website (50), by inputting lists of top 500 upregulated proteins from comparisons between disease conditions and normal in DGE analysis. The top 20 upregulated molecular function GO groups in each comparison with normal were identified and expressed in a heat map (FIG. 20B) color coded by −log 10 q-value B+H (statistical significance obtained from Toppfun analysis). Heat maps were also made to visualize protein expression shifts in specific cytoskeletal groups of interest among disease groups. Morpheus (https://software.broadinstitute.org/morpheus/) was used to generate heat maps. Results of the domain enrichment analysis are available in Table 11.

### Drug Treatment

Viable myocytes were concentrated by gravity (5-7 min) and supernatant was aspirated to remove dead cells. Myocytes were resuspended in NT solution containing 1 mM Ca²⁺ and concentrated by gravity (5-7 min) again. Cardiomyocytes used acutely were maintained in NT solution for up to 6 hours. Myocytes were treated with 10 μM parthenolide (Fisher Scientific NC9013142 or Sigma P0667) or 10 μM colchicine (Sigma) at room temperature in NT solution for 2 hours. Experiments were performed within 6 hours of isolation, except for those involving viral transduction in cultured cells.

NT Solution: NaCl, 140 mM; MgCl₂, 0.5 mM; NaH₂PO₄, 0.33 mM; HEPES, 5 mM; Glucose, 5.5 mM; CaCl₂, 1 mM; KCl, 5 mM; NaOH, pH to 7.4.

### Cell Culture and Viral Transduction

MatTek glass bottom dish was coated 5 μl of 0.5 mg/ml laminin and air dried for 10 min. Culture medium was F-10 (1×) Nutrient Mixture (Ham) [+] L-Glutamine (Life Technologies, 11550-043) supplemented with insulin-transferrin selenium-X (Gibco, 51500-056), 20 mM HEPES, 1 μg/μl primocin (Invivogen, ant-pm-1), 0.4 mM extra CaCl₂, 5% FBS, 25 μM cytochalasin D (Cayman, 11330). Viable myocytes were concentrated and a proper amount of medium was added in culture so that neighboring cells were not in direct contact. Viral constructs were permitted to express for 48 hours with Moi=100-200. Adenoviral TTL-IRES-dsRed and EMTB-3x GFP were transduced in human myocytes as previously performed in rat myocytes. Myocytes were replenished with fresh warm medium without cytochalasin D before calcium and contractility measurements.

### Cell Contractility

Experiments were performed as described in Example 1 with some modification. Contractility is measured in custom-fabricated cell chambers (Ionoptix) mounted on an LSM Zeiss 880 inverted confocal microscope using a 40× oil 1.4 NA objective and transmitted light camera (IonOptix MyoCam-S). Myoyctes were maintained in NT solution (for freshly isolated myocytes) or culture medium (without cytochalasin D, for cultured myocytes) at room temperature and electrical field stimulation was provided at 0.5 Hz with a myopacer (IonOptix MYP100) through platinum electrodes lowered into the bath. Sarcomere length was measured optically by Fourier transform analysis (IonWizard, IonOptix). After 10-30 s of 0.5 Hz pacing to achieve steady state, five traces were recorded and analyzed. If not specified, contractility data was obtained at room temperature.

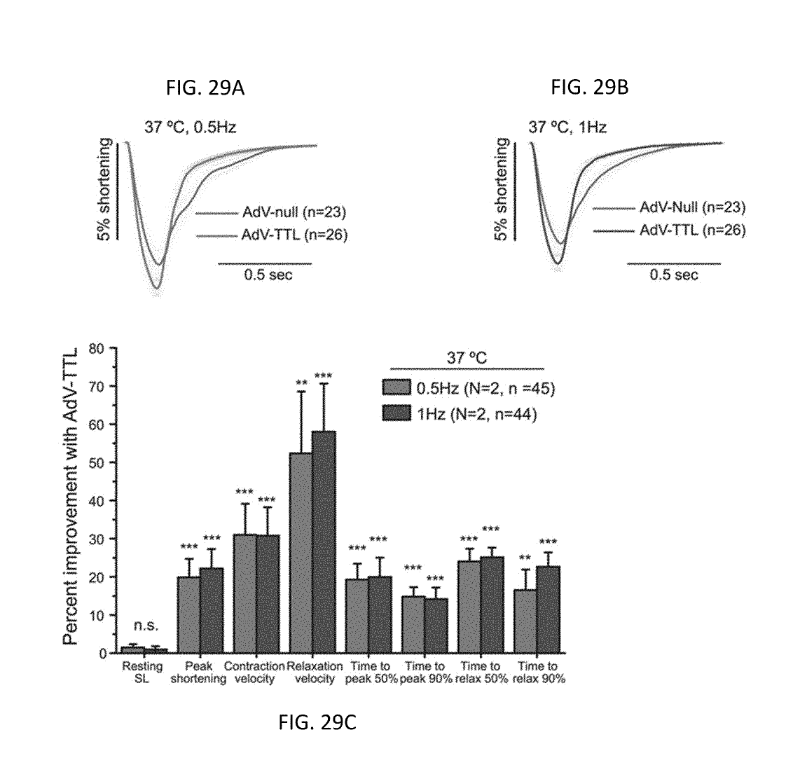

To test whether the contractile improvement over MT destabilization remains under more physiological conditions, both 0.5 Hz and 1 Hz contractions at 37 C were recorded and analyzed in a small subset of isolated human myocytes (1 NF and 1 failing heart, FIG. 29C).

### Calcium Measurements

Calcium measurements were carried out as described in Example 1 with the following modifications. Myocytes were loaded with 1 μM Fluo-3-acetoxymethyl ester (Invitrogen) and 15% Pluronic F127 (a poloxamer made by BASF, Florham Park, N.J., USA) for 15 minutes. Cells were scanned using a 488-nm argon ion laser in confocal line-scan mode at 0.909 ms/line. Cells were electrically paced at 0.5 Hz at room temperature for 30 s to achieve steady state; five steady-state transients of each myocyte were averaged, pooled in groups and analyzed for calcium transient properties. The measured fluorescence (F) throughout the transient was normalized to the resting fluorescence prior to stimulation (F0) to normalize for heterogeneity in dye loading.

### Immunofluorescence

Cells were fixed in pre-chilled methanol at −20° C. for 7 min. After washing with phosphate buffered saline (PBS) for four times, cells were placed in blocking buffer (Seablock, Abcam ab166951) for 1 hour, then labeled with primary antibodies (below) for 48 hours at 4° C. Cells were then washed 4× in blocking buffer, labeled with secondary antibodies (below) at room temperature and washed 3× in blocking buffer. Stained cells were mounted on mouse laminin (Roche 11243217001) coated #1.5 coverslips (22 mm×22 mm, Globe Scientific 1404-15) using Prolong Gold Antifade Mountant (Life Technologies P36934) for imaging.

### Immunofluorescence on Paraffin Tissue Section

Human left ventricle tissues were fixed in 4% paraformaldehyde (PFA) in PBS. Tissues dehydration (reagent alcohol 70% 60 min, 95% 60 min×2, 100% 60 min×3, 37° C.), clearing (xylene 90 min×3, 37° C.), and wax infiltration (110 min and 80 min×2, 62° C.) was automated in a tissue processor (ThermoScientific Excelsior AS). Tissues were then embedded in paraffin on the embedding station. Five-micron tissue sections were cut on a microtome (ThermoScientific Shandon Finesse 325) and mounted on positive charged glass slides (Globe Scientific 1358A).

Slides were placed in a slide rack and rinsed in xylene 2× 3 min for deparaffinization. Tissue sections were re-hydrated in reagent alcohol, 100% 2 min×2, 95% 2 min×2, 70% 2 min, ddH₂O 2 min. Slides were immersed in 1% Tween-20 solution for 5 minutes for permeabilization, then rinsed in diH₂O very briefly, and immersed in 100° C. antigen retrieval buffer (Tris-EDTA buffer, containing 10 mM Tris base, 1 mM EDTA solution, 0.05% Tween 20, pH 9.0.) on a steamer for 40 minutes. Slide chambers with buffer and slides were removed from steamer and allowed to cool at room temperature for 20 minutes. After brief rinsed in ddH₂0, slides were blocked in Seablock for 1 hr at room temperature. Primary antibodies (1:50 in blocking buffer) was incubated over 3 nights at 4° C. Section were rinsed PBS×3 and labeled with secondary antibodies (1:1000) for 1 hr at room temperature. Nuclei were labeled with Hoest 33342 (1:1000) for 10 min. Slides were rinsed with PBS for 4 times, blot dry, mounted using Prolong Diamond Antifade mounting medium (Thermo Fisher Scientific, #P36965) for imaging.

### Western Blotting

For analysis of proteins expression levels quantitative western blots (WB) were performed using infrared fluorescence imaging on an Odyssey Imager (LI-COR). Human left ventricular tissue homogenates were prepared in ice-cold RIPA lysis buffer containing 0.1% SDS (Cayman 10010263) supplemented with protease inhibitor cocktail (Sigma). In some of the homogenates, lysis buffer also contained protease/phosphatase inhibitor cocktail (Cell signaling 5872). Protein concentration was determined by protein assay dye reagent (Bio-Rad). Aliquots of supernatants were mixed with 4× sample buffer (LI-COR, 928-40004) containing 10% BME, boiled for 10 min, and resolved on sodium dodecyl sulfate (SDS)-polyacrylamide gel electrophoresis Tris-glycine gels (Bio-Rad). Proteins were transferred to a membrane on Mini Trans-Blot Cell (Bio-Rad), blocked 1 hour in Odyssey Blocking Buffer (TBS) (LI-COR, 927-50000), and probed with the corresponding primary antibodies (see list below) for overnight at 4° C. Membranes were then rinsed with TBS containing 0.05% Tween (TBST) 20 4× for 5 min, and incubated with secondary antibodies (below) in TBS supplemented with 0.2% Tween20 for 1 hour at room temperature. Membranes were rinsed again with TBST and then imaged on Odyssey Imager. Image analysis was performed using Image Studio Lite software (LI-COR). GAPDH was used as loading control. We also controlled for variability of different experiments/blots by including 3 reference samples from normal group in every blot Fluorescent band intensity was normalized to GAPDH. Before pooling data from different blots, the values were again normalized to the average of the 3 reference samples. Analysis was performed blinded to the experimental groups.

### Antibodies and Labels

Alpha tubulin; mouse monoclonal, clone DMIA (Cell Signaling #3873)—isolated myocyte IF 1:100, tissue section IF 1:50 (primary labeled Atto- or dylight-488, Innova Biosciences, 322-0010, 350-0010); WB 1:1000
Tyrosinated tubulin; mouse monoclonal, clone TUB-1A2 (Sigma T 9028); isolated myocyte IF 1:200, WB 1:1000
Detyrosinated tubulin; rabbit polyclonal (abcam ab48389); isolated myocyte IF 1:200; WB 1:1000
Tubulin tyrosine ligase; rabbit polyclonal (proteintech 13618-1-AP); WB 1:500
GAPDH; mouse monoclonal, clone 3B1E9 (GenScript A01622-40); WB 1:1000
Desmin; goat polyclonal (R&D AF3844); IF 1:100 (primary labeled Atto 565, Innova Biosciences)
Goat anti-mouse AF 647 (Life Technologies, A-21235); IF 1:500
Goat anti-rabbit AF 488 (Life Technologies, A-11034); IF 1:500

### IRDye800CW (LI-COR, #925-32210); WB 1:10000

### IRDye680RD (LI-COR, #925-68071); WB 1:10000

Lightning Link Rapid Atto565 (Innova Biosciences, 351-0030), Atto 488 (322-0010) and dylight 488 (350-0010)
Hoechst 33342 (1:1000) for labeling nuclei

### Imaging Equipment and Analysis

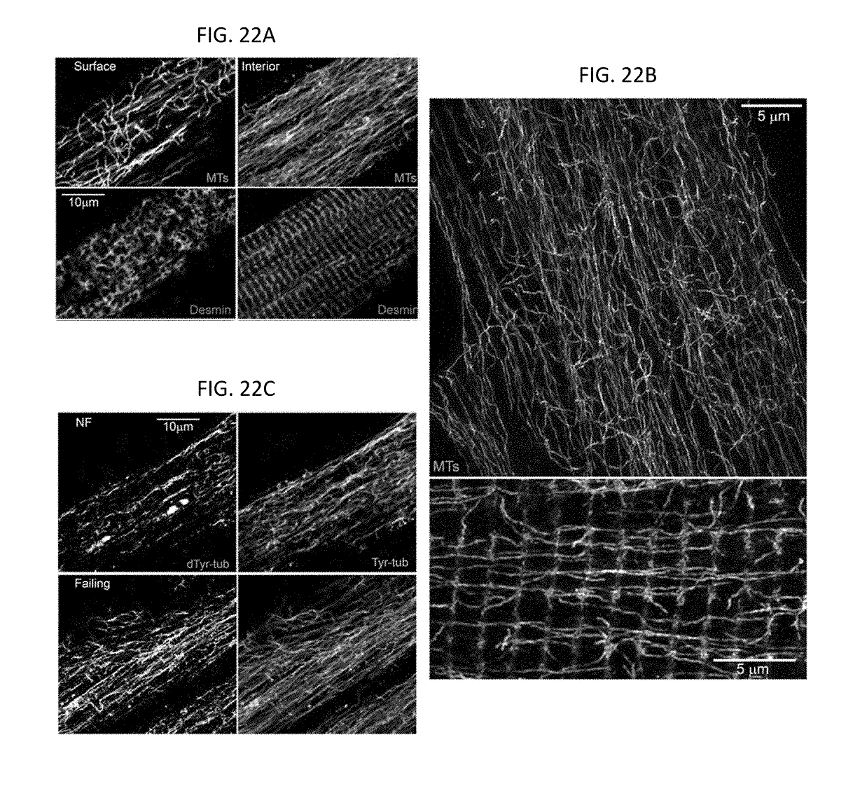

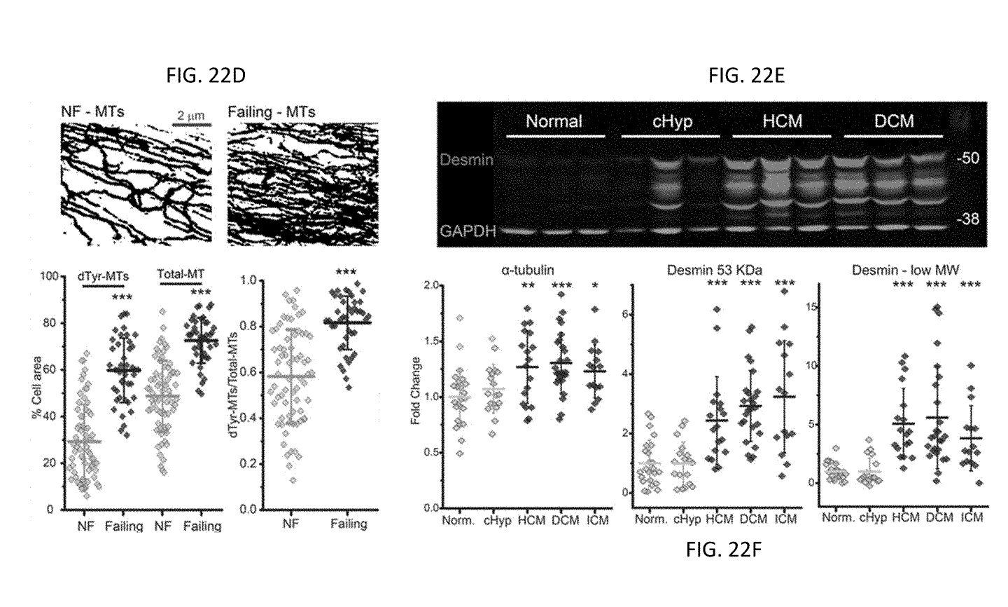

Confocal imaging was carried out on a Zeiss 880 laser scanning confocal microscope operating on an Axiovert Z1 inverted microscope equipped with a 40× oil 1.4 NA objective. High-speed super resolution imaging was carried out on a Zeiss 880 Airyscan confocal with a 40×1.4 oil NA objective. Image analysis was performed using ZEN Black software for Airyscan processing, which involves signal integration from the 32 separate sub-resolution detectors in the Airyscan detector and subsequent deconvolution of this integrated signal. Additional signal processing was performed in Image J (NIH). For calculating the microtubule (MT) fraction of cell area (FIG. 22D), image analysis was performed blinded to the experimental groups. A 2-μm max intensity projection was produced from four 0.5-μm confocal z-sections. The threshold to determine MT positive pixels was determined from the average fluorescence of three background regions that clearly demonstrated no MT staining within the cell. From the resulting binary images (FIG. 22D top), the MT positive fraction of the total cell area was calculated for dTyr-MT and Tyr-MT network (FIG. 22D bottom). Then binary images of both dTyr- and Tyr-MT channels were overlaid to generate a binary image of the total MT network of the cell, which enables the quantification of total-MT area and ratio of dTyr-MT/total MT (FIG. 22D bottom).

Structured Illumination Microscopy (SIM) was performed on a Deltavison OMX microscope, equipped with a Front Illuminated sCMOS camera (2560×2160 pixels), 6 color solid state illuminator and 100× 1.4 NA oil immersion lens powered by a OMX Master Workstation.

### Nanoindentation—Stiffness Measurements

Mechanical properties at the microscopic scale were measured using nanoindentation (Piuma, Optics11, The Netherlands). Freshly isolated human myocytes were attached to glass bottom dish coated with MyoTak (1) in NT solution (1 mM Ca²⁺) at room temperature. A spherical nano-indentation probe with a radius of 3.05 μm and a stiffness of 0.026 N/m was used. Myocytes were indented to a depth of 1.5-3.5 μm with velocities of 0.1, 0.25, 0.5, 1.0, 2.0, 5.0, 10.0, 20.0, 50.0, 100.0, and 150.0 μm/s. The tip was held in this indentation depth for 1 s, and retracted over 2 s. The Young's moduli were calculated automatically by the software, by fitting the force vs. indentation curve to the Hertz equation. The Young's modulus E is derived from the fit of the initial 60% of the loading force-displacement curve (F(h)), the indenter tip radius (R) and indentation depth (h), according to the following formula, for which a Poisson's ratio (ν) of 0.5 was assumed.

$$F ( h ) = \frac{4}{3} \frac{E}{( 1 - {v}^{2} )} {R}^{1 / 2} {h}^{3 / 2}$$

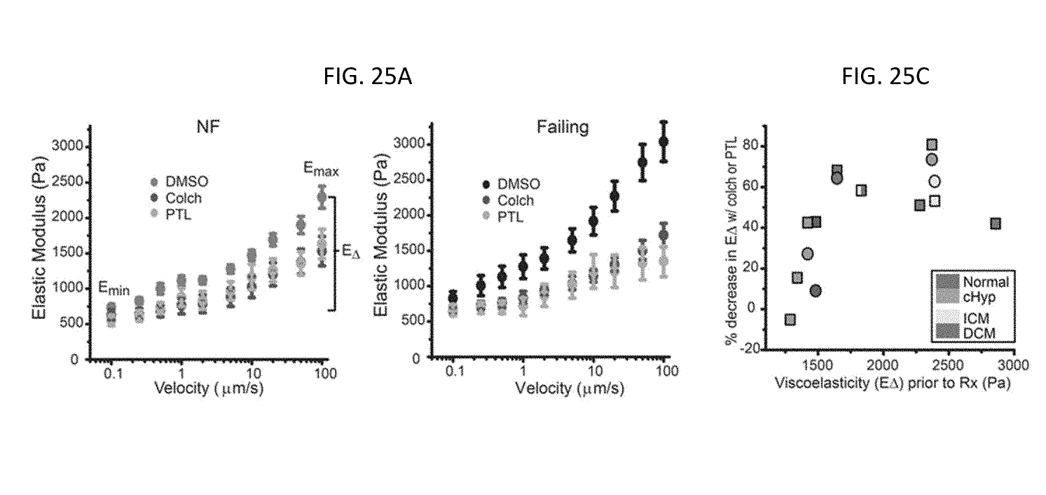

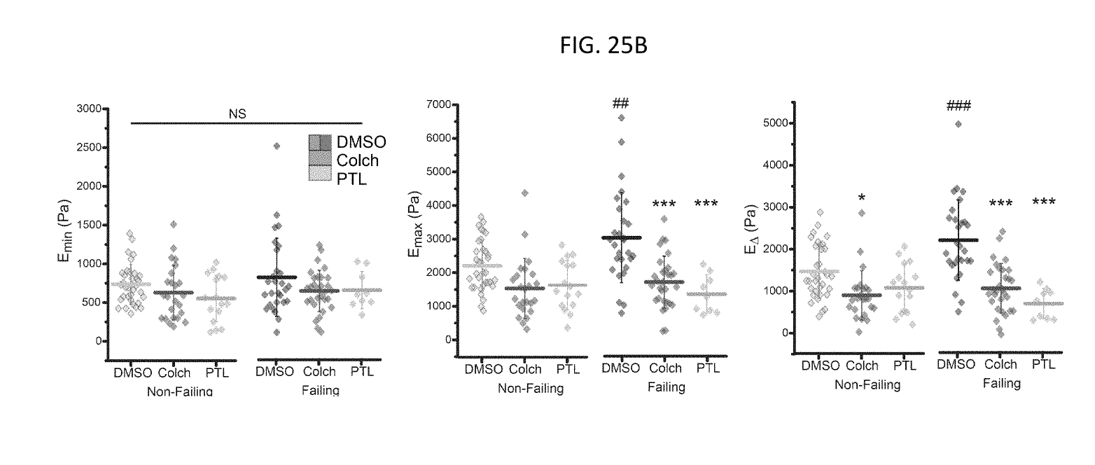

Average of E in each condition with standard error is plotted against different indentation speed (FIG. 25A). Low velocity indentation measures elastic contributions (Eₘᵢₙ) to stiffness, high velocity indentation measures elastic and viscous contributions (Eₘₐₓ). The change in modulus with rate represents myocyte viscoelasticity (EΔ). Quantification of Eₘᵢₙ, Eₘₐₓ and EΔ is shown in FIG. 25B.

### Data Handling and Statistics

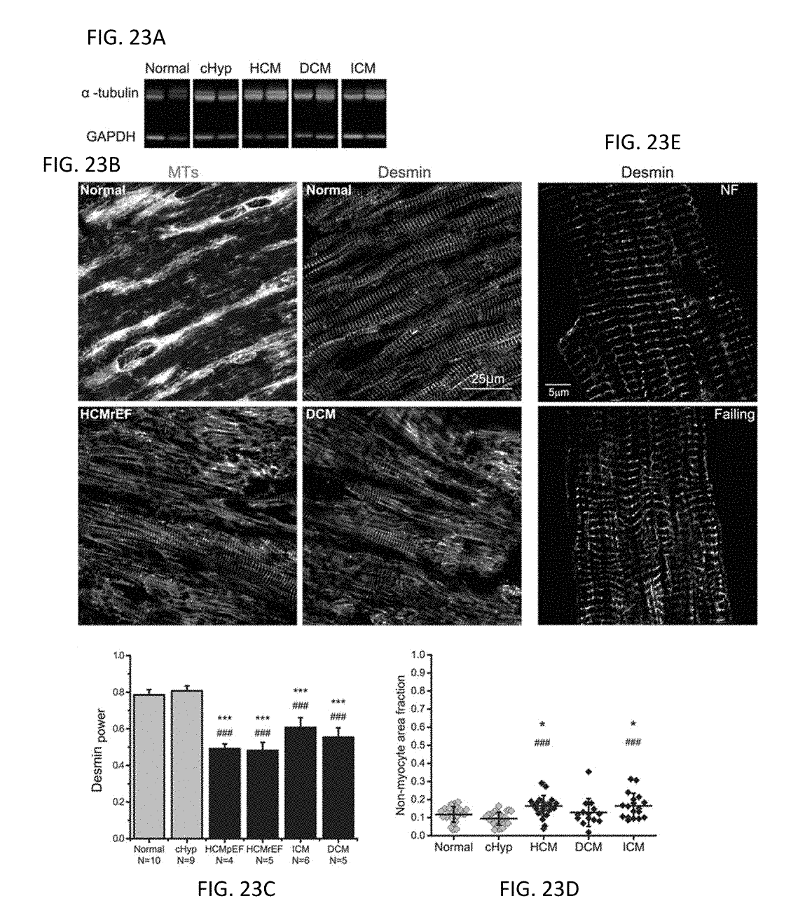

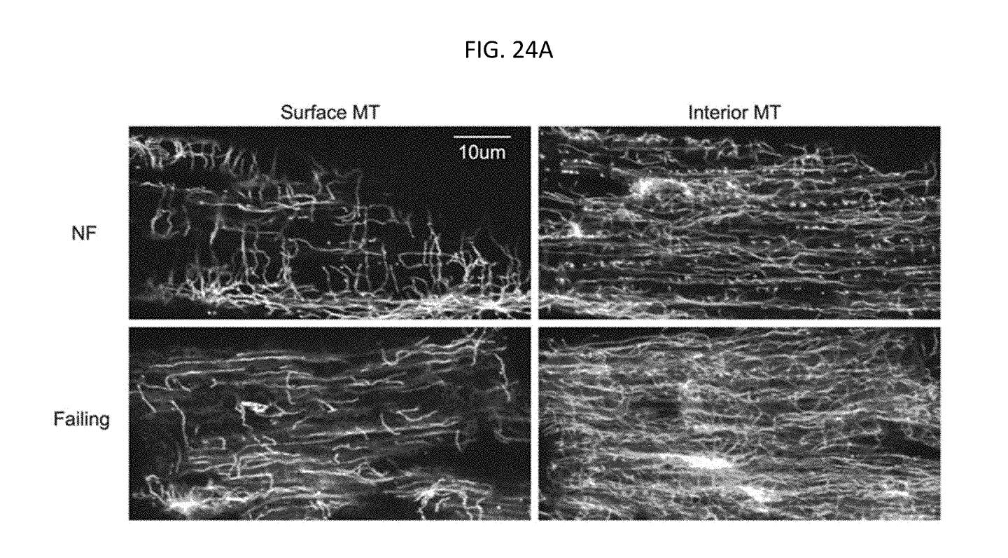

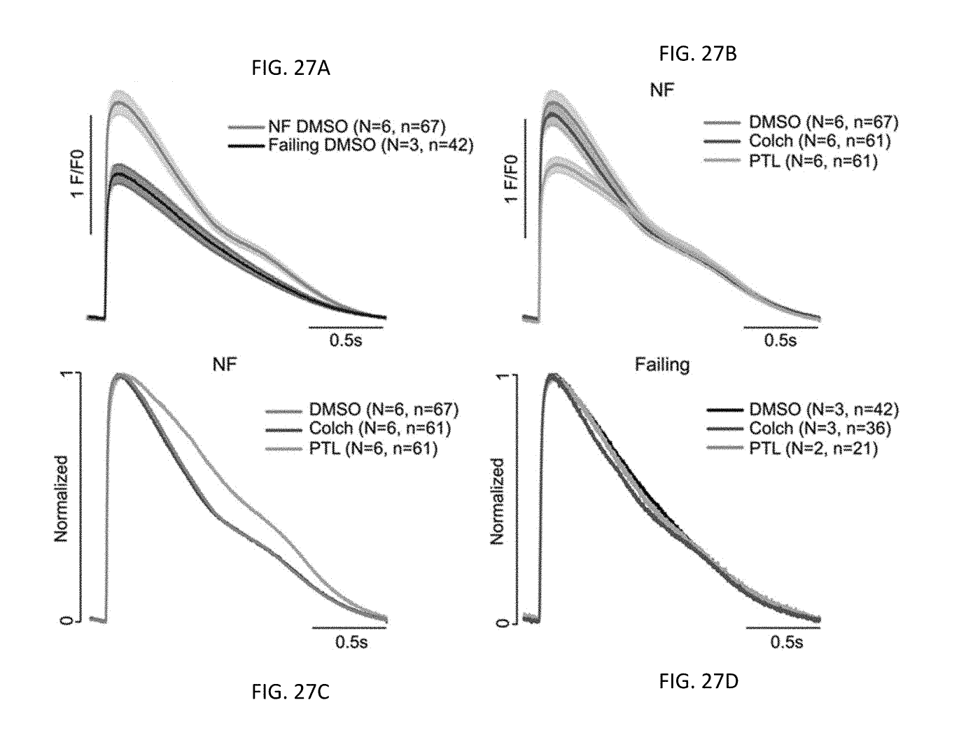

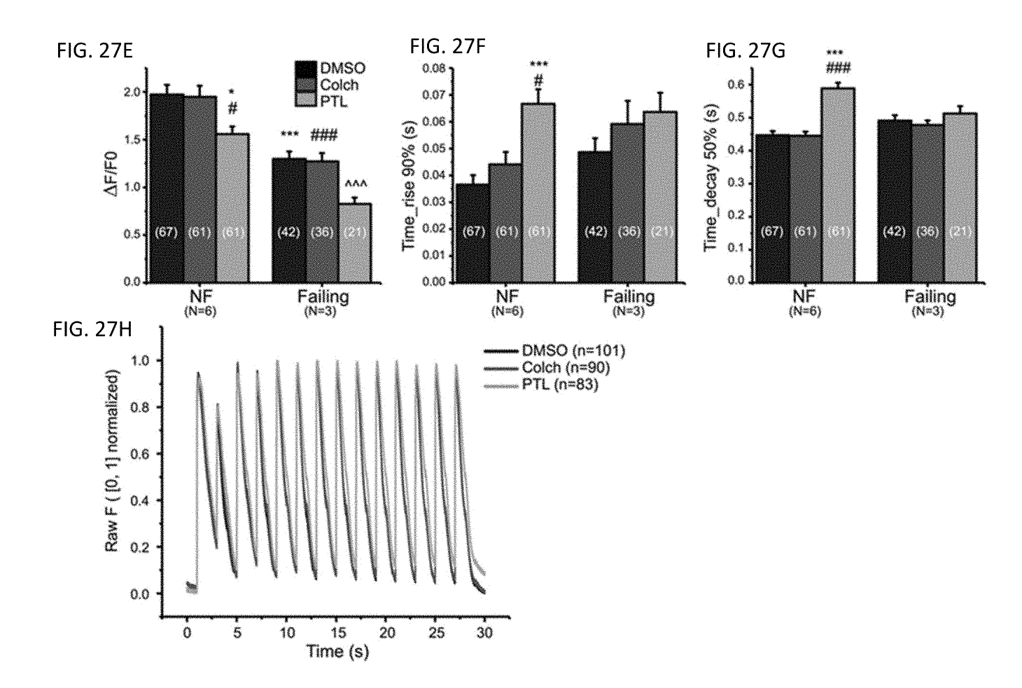

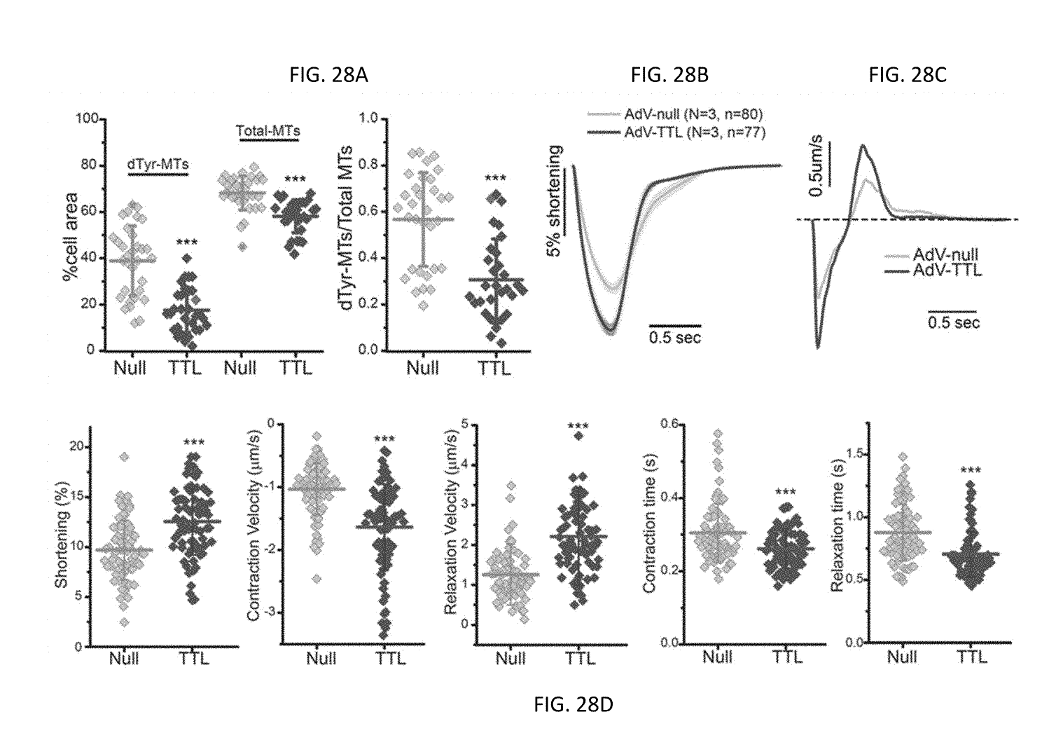

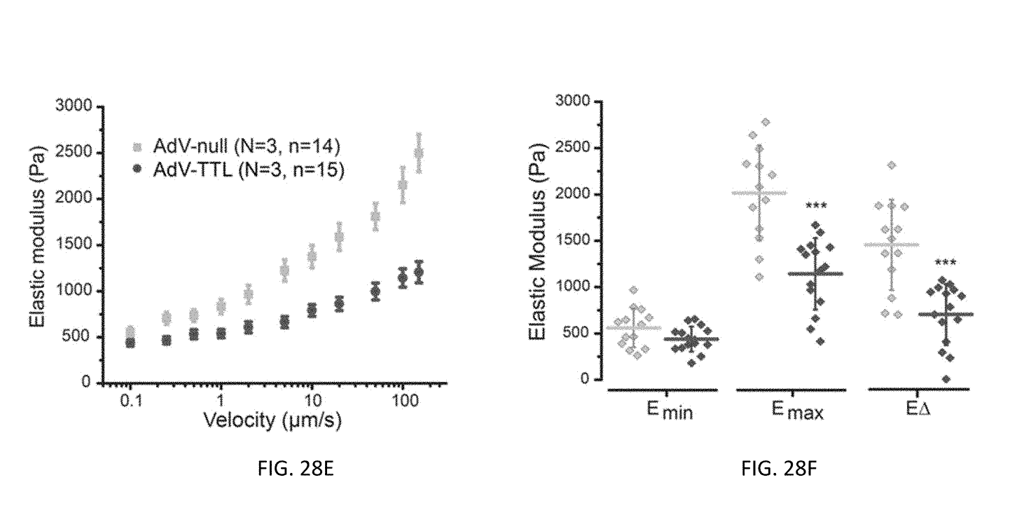

Statistical analysis and graphing were performed using Origin software (OriginLab, Northampton, Mass.). Values are presented as means±standard error in bar and line graphs; medians are presented as a line in dot plots; box plots represent 25 to 75 percentile with whiskers ranging means±standard deviation. Where comparisons between sets were both repetitive and restricted, the Bonferroni multiple comparisons correction was used to adjust the significance threshold of two-sided T-tests accordingly (FIG. 22D-FIG. 22F; FIG. 28A, FIG. 28D, FIG. 28F). Multiple comparisons—ANOVA with post-hoc Tukey test was used when multiple data sets shared a single control condition (FIG. 23C and FIG. 23D; FIG. 25B; FIG. 27E-FIG. 27G). Outlier exclusion was applied to immunofluorescence quantification data (FIG. 22D) exceeding 2 standard deviations from the mean. No exclusion was applied to other data sets.

### Results

This study utilized left ventricular (LV) myocardium from 105 non-failing (NF) and failing human hearts. NF hearts are subdivided into normal or compensated hypertrophy (cHyp), while failing hearts are subdivided into ischemic cardiomyopathy (ICM), dilated cardiomyopathy (DCM), and hypertrophic cardiomyopathy with preserved or reduced ejection fraction (HCMpEF and HCMrEF, respectively). Western blot was performed on 102 hearts; 34 of these were also used for mass spectrometry, while 22 were used for primary isolation of cardiomyocytes for functional studies.

### Conserved Upregulation and Stabilization of MTs and IFs Across Human Heart Failure

First we sought to globally characterize changes to the cytoskeletal proteome that occur in human heart disease. Tandem mass spectrometry (MS) analysis detected 3,764 proteins in LV tissue, and there was a distinct proteomic distribution based on patient etiology (FIG. 20A, FIG. 21B-FIG. 21E). Principal component analysis (PCA) was used to examine and display the variance in protein distribution among groups, and demonstrates that NF hearts are well separated from failing hearts. Among failing hearts, the proteomic profiles of non-ischemic heart disease (HCM and DCM) cluster tightly together, while those from ischemic hearts demonstrate a distinct proteomic profile. Non-myocyte populations will contribute to these proteomic profiles, but given their small contribution to tissue mass and modest changes in this contribution observed in disease samples (FIG. 23D) (49), the dominant shifts in myocyte proteins likely arise from within that population.

We ranked the protein list for each patient population based on the significance of the change in protein expression compared to normal hearts. The top 25 up- and down-regulated proteins from each patient group are displayed in Table 10. Gene Ontology (GO) analysis (50) was used to determine the most robustly changed molecular functions in each patient population relative to normal hearts (FIG. 20B). In cHyp, the most prominent increases were in GO groups related to mitochondrial function or redox balance. In ICM, significant upregulation in endopeptidases and proteins involved in immune and inflammatory responses were observed. Yet in non-ischemic heart failure, particularly in HCMrEF and DCM, 3 of the 5 most significantly upregulated GO groups encoded cytoskeletal proteins (FIG. 20B). This pattern is also evident in protein domain analysis, where tubulin, spectrin, and CH-type domains are the most prominently increased in non-ischemic (but not ischemic) heart failure (Table 11).

TABLE 11
Top 10 domains in functional enrichment analysis

| | | Hit Count in | |
|---|---|---|---|
| | q-value | Query List/ | |
| Domain | FDR B&H | Genome | Hit in Query List |
| cHyp vs. normal | | | |
| Thioredoxin-like_fold | 1.27E−06 | 19/132 | TXN, TXNRD1, PTGES2, MIEN1, GSTK1, ERP44, |
| | | | PRDX3, GLRX, ERP29, GPX1, CASQ2, PDIA3, |
| | | | TXNDC12, GSTM1, GSTM2, GSTM3, GSTZ1, |
| | | | PDIA6, P4HB |
| NAD(P)-bd_dom | 1.30E−04 | 18/169 | UBA5, ME1, UBA1, H6PD, CRYM, CRYZ, G6PD, |
| | | | GAPDH, BDH2, BDH1, GLUD2, MTHFD2L, |
| | | | MAT2B, RTN4IP1, SCCPDH, EHHADH, AASS, |
| | | | HSD17B10 |
| 2-oxoacid_DH_actylTfrase | 1.30E−04 | 4/4 | DBT, DLAT, DLST, PDHX |
| FAD/NAD-linked_Rdtase_dimer | 1.30E−04 | 5/8 | TXNRD1, TXNRD2, DLD, TXNDC12, GSR |
| Spectrin/alpha-actinin | 1.94E−04 | 7/23 | ACTN4, ACTN1, ACTN2, SPTAN1, DMD, SYNE1, |
| | | | MACF1 |
| Ald_DH_CS_CYS | 3.05E−04 | 6/17 | ALDH1B1, ALDH9A1, ALDH3A2, ALDH6A1, |
| | | | ALDH4A1, ALDH1L1 |
| EF-hand_Ca_insen | 4.40E−04 | 4/6 | ACTN4, ACTN1, ACTN2, SPTAN1 |
| 26S_Psome_P45 | 4.40E−04 | 4/6 | PSMC2, PSMC3, PSMC4, PSMC5 |
| Tubulin_C | 5.26E−04 | 6/20 | TUBA4A, TUBA1B, TUBB4B, TUBB6, TUBA1A, |
| | | | EHHADH |
| THDP-binding | 7.27E−04 | 5/13 | PDHA1, PDHB, OGDHL, BCKDHA, ILVBL |
| HCMpEF vs. normal | | | |
| EF-hand_Ca_insen | 7.83E−05 | 5/6 | ACTN4, ACTN1, ACTN2, SPTA1, SPTAN1 |
| Spectrin_repeat | 3.23E−04 | 8/29 | ACTN4, ACTN1, ACTN2, PLEC, SPTA1, SPTAN1, |
| | | | SPTBN1, DMD |
| MACPF_CS | 1.52E−03 | 4/6 | C6, C7, C8A, C8B |
| Ig_C1-set | 1.85E−03 | 9/54 | HLA-A, HLA-B, HLA-C, HLA-DRA, HLA- |
| | | | DRB1, IGLL5, IGHA1, IGHG1, IGHG4 |
| CH | 2.06E−03 | 10/70 | ACTN4, ACTN1, ACTN2, PLEC, PLS3, MAPRE3, |
| | | | PARVA, SPTBN1, IQGAP1, DMD |
| EF-hand_1 | 4.06E−03 | 14/152 | ACTN4, ACTN1, ACTN2, RYR2, EHD3, S100A6, |
| | | | PLS3, VSNL1, SLC25A12, SPTAN1, PDCD6, |
| | | | CAPN1, CAPN2, SLC25A13 |
| Sarcoglycan | 6.06E−03 | 3/4 | SGCB, SGCD, SGCG |
| Ribosomal_L23/L15e_core_dom | 6.06E−03 | 3/4 | RPL23A, RPS24, RPL15 |
| Tubulin_C | 1.01E−02 | 5/20 | TUBA1B, TUBB, TUBB8, TUBB6, TUBA1A |
| EF-hand-dom_pair | 1.08E−02 | 18/261 | ACTN4, ACTN1, ACTN2, RYR2, EHD4, EHD3, |
| | | | S100A6, PLS3, VSNL1, SLC25A12, SPTA1, |
| | | | SPTAN1, DMD, PDCD6, DTNA, CAPN1, CAPN2, |
| | | | SLC25A13 |
| HCMrEF vs. normal | | | |
| Tubulin_C | 3.50E−07 | 9/20 | TUBA4A, TUBB2A, TUBA1B, TUBB4B, TUBB, |
| | | | TUBB8, TUBB6, TUBA1A, EHHADH |
| CH | 3.50E−07 | 14/70 | ACTN4, ACTN1, ACTN2, PLEC, PLS3, CNN3, |
| | | | FLNA, MAPRE3, PARVA, SPTB, SPTBN1, IQGAP1, |
| | | | DMD, MAPRE2 |
| Actinin_actin-bd_CS | 3.50E−07 | 9/23 | ACTN4, ACTN1, ACTN2, PLEC, PLS3, FLNA, |
| | | | SPTB, SPTBN1, DMD |
| Spectrin_repeat | 2.82E−06 | 9/29 | ACTN4, ACTN1, ACTN2, PLEC, SPTA1, SPTAN1, |
| | | | SPTB, SPTBN1, DMD |
| EF-hand_Ca_insen | 5.76E−06 | 5/6 | ACTN4, ACTN1, ACTN2, SPTA1, SPTAN1 |
| Beta-tubulin_BS | 1.85E−04 | 5/10 | TUBB2A, TUBB4B, TUBB, TUBB8, TUBB6 |
| Vinculin/catenin | 9.99E−04 | 4/7 | VCL, CTNNA2, TLN2, TLN1 |
| Alpha_tubulin | 3.35E−03 | 4/9 | TUBA4A, TUBA1B, TUBA1A, EHHADH |
| Small_GTPase | 4.14E−03 | 14/160 | RRAS, RRAS2, ARF1, ARF3, RHOA, RHOC, |
| | | | RHOG, RAB10, RAB1A, RAB4A, RAC1, RAP1A, |
| | | | RAP1B, CDC42 |
| C1-set | 4.14E−03 | 8/54 | HLA-B, HLA-DRA, HLA-DRB1, IGLL5, IGHA1, |
| | | | IGHG1, IGHG3, IGHG4 |
| DCM vs. normal | | | |
| Small_GTP-bd_dom | 1.36E−04 | 19/167 | RRAS, RRAS2, ARF1, ARF3, RHOA, RHOC, |
| | | | RAB1A, RAB2A, RAB4A, RAB5B, RAB6A, |
| | | | RAB5C, RAC1, RAN, RAP1A, RAP1B, DRG2, |
| | | | RHOQ, CDC42 |
| CH | 1.36E−04 | 12/70 | ACTN4, ACTN1, PLEC, PLS3, FLNA, MAPRE3, |
| | | | PARVA, LIMCH1, SPTBN1, IQGAP1, DMD, |
| | | | MAPRE2 |
| Tubulin_C | 1.38E−04 | 7/22 | TUBA4A, TUBA1B, TUBB4B, TUBB, TUBB8, |
| | | | TUBB6, TUBA1A |
| Actinin_actin-bd_CS | 1.38E−04 | 7/23 | ACTN4, ACTN1, PLEC, PLS3, FLNA, SPTBN1, |
| | | | DMD |
| EF-hand_Ca_insen | 5.70E−04 | 4/6 | ACTN4, ACTN1, SPTA1, SPTAN1 |
| Spectrin_repeat | 6.02E−04 | 7/29 | ACTN4, ACTN1, PLEC, SPTA1, SPTAN1, |
| | | | SPTBN1, DMD |
| LIM | 8.59E−04 | 10/69 | FHL1, CRIP2, CSRP1, LIMCH1, PDLIM3, |
| | | | LASP1, CSRP3, LIMS1, PDLIM1, LPP |
| ANATO | 8.59E−04 | 4/7 | FBLN1, FBLN2, C3, C4B |
| Sarcoglycan | 3.73E−03 | 3/4 | SGCB, SGCD, SGCG |
| Beta_tubulin | 4.32E−03 | 4/10 | TUBB4B, TUBB, TUBB8, TUBB6 |
| ICM vs. normal | | | |
| ANATO | 8.16E−05 | 5/7 | FBLN1, C3, C4A, C4B, C5 |
| Serpin_CS | 2.32E−04 | 8/32 | SERPINF1, SERPINA4, SERPINF2, SERPINC1, |
| | | | SERPING1, SERPINA7, SERPINA6, SERPIND1 |
| Anaphylatoxn_comp_syst_dom | 2.32E−04 | 4/5 | C3, C4A, C4B, C5 |
| A2M_comp | 2.32E−04 | 5/10 | A2M, C3, C4A, C4B, C5 |
| Thioredoxin-like_fold | 2.32E−04 | 14/122 | SH3BGRL, MIEN1, ERP44, UBXN4, TXNDC5, |
| | | | GLRX, ERP29, GPX1, CASQ2, PDIA3, TXNDC12, |
| | | | GSTM1, PDIA6, P4HB |
| Sushi | 3.95E−04 | 9/52 | CFH, APOH, VCAN, CFB, C1R, C1S, C6, C7, |
| | | | SUSD2 |
| EF-hand_Ca_insen | 3.95E−04 | 4/6 | ACTN4, ACTN1, ACTN2, SPTAN1 |
| MACPF_CS | 3.95E−04 | 4/6 | C6, C7, C8A, C8B |
| LIM | 5.15E−04 | 10/69 | FHL1, LIMS3, CRIP1, CSRP1, PDLIM3, LASP1, |
| | | | CSRP3, LDB3, PDLIM1, LMO7 |
| Nucleotide-bd_a/b_plait | 6.59E−04 | 19/244 | RPL23A, MRPL23, HNRNPA2B1, HNRNPC, |
| | | | HNRNPD, HNRNPH3, CIRBP, SYNCRIP, |
| | | | SRSF3, SRSF7, FUS, SNRNP70, MATR3, |
| | | | HNRNPM, NCL, HNRNPA0, HNRNPLL, RBM3, |
| | | | HNRNPR |

We further examined the expression profiles of the individual proteins driving these cytoskeletal changes. FIG. 20C demonstrates protein expression of the major cytoskeletal sub-groups—IF proteins, tubulin, and actin/myosin. IF proteins show a pronounced and progressive upregulation from cHyp to end-stage heart failure. The major tubulin isotypes are also progressively, but more modestly, increased. Conversely, most actin and myosin isoforms, including sarcomeric actomyosin, tend to decrease in relative abundance in disease.

In FIG. 20D we highlight several specific proteins of interest. In end-stage failing hearts, many of the most upregulated proteins are cytoskeletal in nature. Thrombospondin-4 (gene:THBS4) and supervilin (gene: SVIL) are involved in linking the plasma membrane to the external matrix and to the internal cytoskeleton (51, 52). Microtubule associated protein 4 (gene: MAP4) is the predominantly expressed MAP in the heart. It can bind and stabilize cardiac MTs, as well as promote dTyr and resistance to myocyte contraction (53, 54). MAP4 is increased in every patient sample compared to any normal control, suggesting a highly-conserved upregulation of this MT stabilizer in human heart disease. Finally, while most tubulin isoforms are synthesized with a C-terminal tyrosine, α-tubulin A4A (gene: TUBA4A) is synthesized in its detyrosinated form (55). Thus, increases in both MAP4 and TUBA4A protein are predicted to increase the amount of stable, detyrosinated MTs in failing human hearts.

Together, this proteomic analysis suggests that the upregulation and stabilization of the cytoskeleton—specifically MTs, IFs, and proteins associated with linking the cytoskeleton to the external environment—is a prominent feature of end-stage heart failure in humans.

### Proliferation and Modification of MTs and IFs in Diseased Human Myocytes

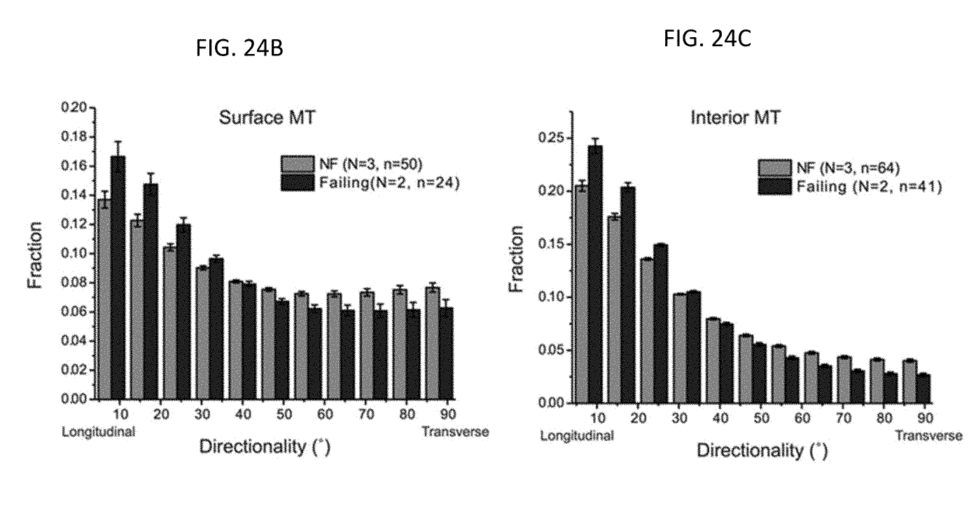

To validate and extend our proteomic results, we performed quantitative western blot and immunofluorescence analysis of cytoskeletal targets in human myocardium and myocytes. As MTs tend to fragment during fixation of myocardial tissue (See Methods, FIG. 23B), we examined the network organization in isolated LV myocytes using super-resolution imaging. Just under the membrane, cortical MTs are chaotically organized and show frequent transverse elements, while deeper into the cell the network becomes quite dense, with interior MTs predominantly aligned along the long axis of the myocyte (FIG. 22A). This organization is mostly conserved between failing and NF myocytes (FIG. 24), in contrast to the network disorganization seen in myopathies that arise from the loss of costameric proteins (56). Structured illumination microscopy (SIM) of the interior MTs in a normal human myocyte reveals an intricate network, predominantly formed by single or paired MTs running tens of microns in the cell, forming a lattice like structure with transverse desmin filaments (FIG. 22B). As observed in murine myocytes, these longitudinal MTs buckle predominantly between sarcomeric Z-disks during contraction, suggesting that they bear compressive load. This periodic buckling was observed in both non-failing and failing cardiomyocytes.

The MT network is highly proliferated and detyrosinated in failing vs. NF myocytes (FIG. 22C and FIG. 22D). Quantitative image analysis reveals that the fraction of cell area covered by all MTs or dTyr-MTs increases by 1.5 and 2-fold respectively in failing myocytes (FIG. 22D, bottom left), with a significant increase in the ratio of dTyr to total MTs (FIG. 22D, bottom right). This increase in MT density is concomitant with an increase in total tubulin protein, as western blotting of LV tissue shows a significant upregulation of α-tubulin in HCM, DCM, and ICM (FIG. 22F, FIG. 23A).

In addition, failing myocytes demonstrate a distorted arrangement of desmin and misaligned myofibrils (FIG. 23B, FIG. 23C, and FIG. 23E). Quantification of desmin organization shows reduced periodicity in failing LV myocardium, but not in cHyp hearts (FIG. 23C). Western blot analysis demonstrates a marked upregulation of desmin in human heart failure (FIG. 22E and FIG. 22F). Interestingly, we observed multiple bands below desmin's predicted molecular weight (MW); some of these low MW bands were previously identified as post-translationally modified desmin products that are prone to misfolding, aggregation, and cleavage (57). These low MW products were increased above the NF mean in 59 out of 60 failing hearts (FIG. 23E and FIG. 22F).

### MTs Increase Viscoelasticity in Failing Myocytes

Next we sought to test whether detyrosinated MTs differentially affect the mechanical properties of failing vs. NF myocytes. To assess passive mechanics, viscoelasticity was measured via transverse nano-indentation and variable indentation-rate viscoelastic analysis (VIVA) (43). Briefly, myocytes are indented at progressively increasing rates to evaluate elastic as well as viscoelastic contributions to myocyte stiffness. As seen in FIG. 25A, stiffness increases as a function of indentation rate, demonstrating that human myocytes are indeed viscoelastic. The Elastic modulus (E) at the lowest velocity arises primarily from elastic components within the myocyte (Eₘᵢₙ), while high velocity stiffness reflects both elastic and viscous contributions (Eₘₐₓ). The change in modulus with rate is a useful indicator of viscoelasticity (EΔ). Failing myocytes showed no difference in Eₘᵢₙ (FIGS. 25A and 25B left Eₘᵢₙ, DMSO-treated cells), but were significantly more viscoelastic than NF myocytes (FIG. 25A and FIG. 25B Eₘₐₓ and EΔ, DMSO-treated cells).

Next, we tested the contribution of MTs and MT detyrosination to this increased viscoelasticity. Parthenolide (PTL) inhibits the detyrosinating enzyme tubulin carboxypeptidase (TCP), which catalyzes the removal of the C-terminal tyrosine from α-tubulin. Ten μM PTL suppresses detyrosination in cardiac and skeletal muscle without grossly disrupting MT density (13), while 10 μM colchicine (colch) broadly depolymerizes MTs (8). No differences in elasticity were observed between NF and failing myocytes in any treatment condition (FIG. 25A and FIG. 25B left, Eₘᵢₙ). While MT destabilization had a modest effect on normal myocytes, both colch and PTL treatment robustly reduced viscoelasticity in failing myocytes (FIG. 25B, Eₘₐₓ and EΔ), indicating that the proliferation of dTyr-MTs increases transverse stiffness in human heart failure. FIG. 25C provides a summary of these studies, plotting the drug-induced decrease in viscoelasticity relative to the initial stiffness of each heart tested. In general, stiffer myocytes show larger reductions in viscoelasticity after destabilizing MTs or suppressing detyrosination.

dTyr-MTs Impede Contractility in Failing Myocytes

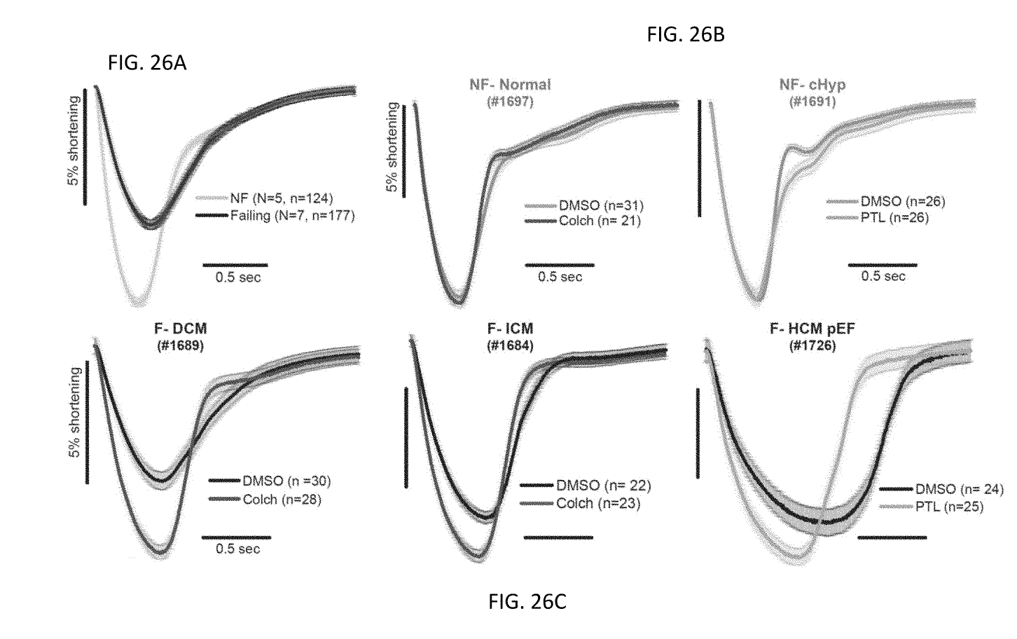

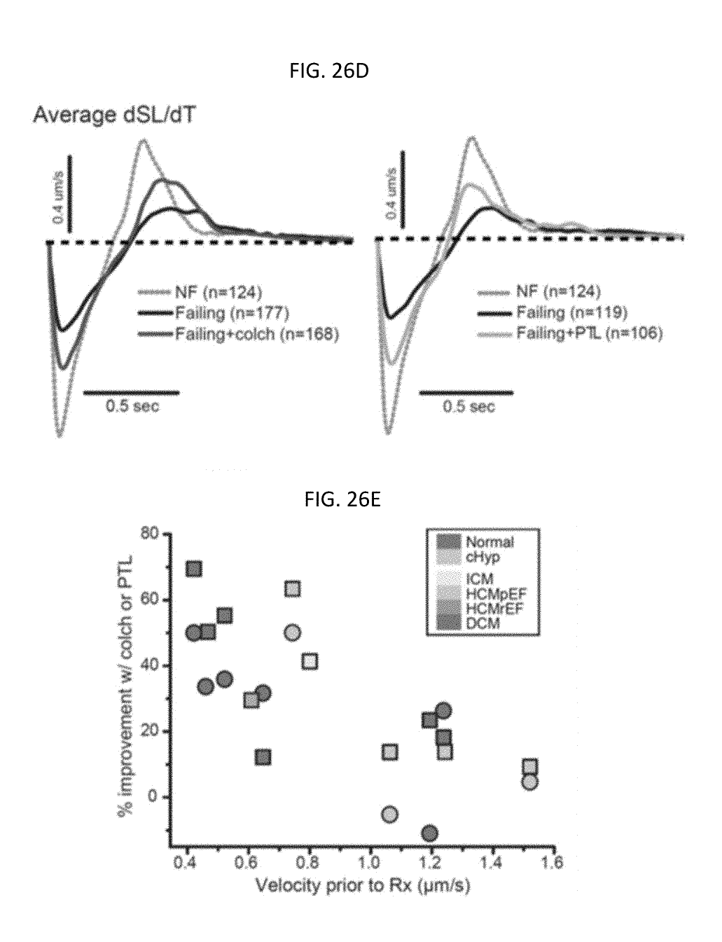

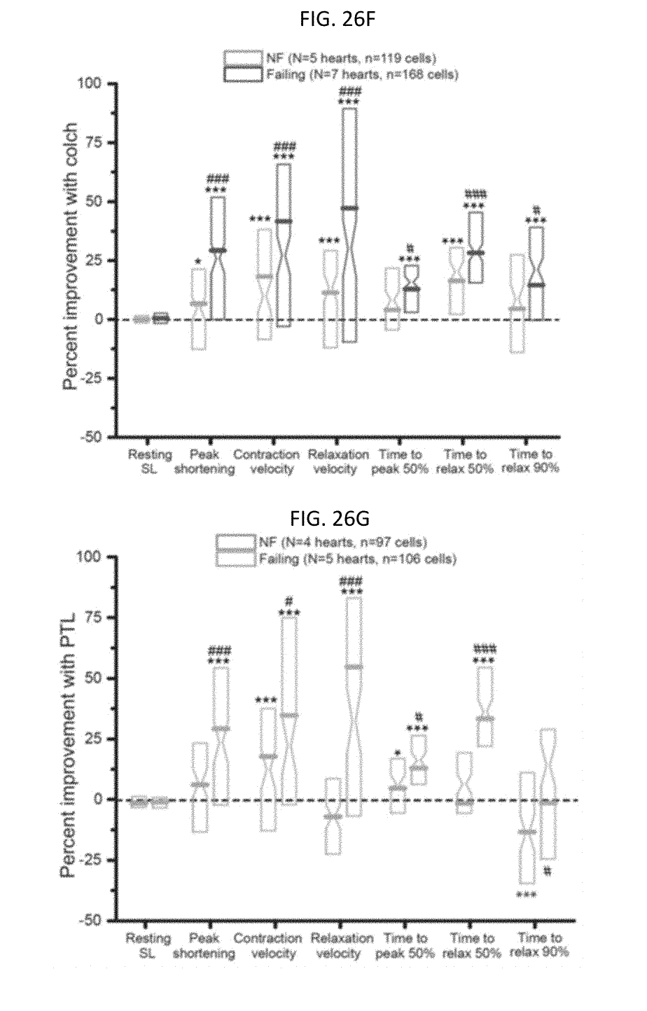

If dTyr-MTs provide viscoelastic resistance, removing them should reduce this resistance, improving contractility. Thus we assessed sarcomere length (SL) and contractile velocities during electrical stimulation of 785 freshly isolated myocytes from 12 human hearts, 7 failing and 5 NF. Prior to treatment, failing myocytes demonstrated markedly reduced sarcomere shortening, with slower contraction and relaxation velocities compared to NF cells (FIG. 26A and FIG. 26E). FIG. 26B and FIG. 26C depict average traces of myocyte shortening with and without MT destabilization from 5 representative NF and failing hearts of different etiology. On average, NF myocytes treated with colchicine showed modest (yet significant) increases in shortening amplitude and contractile velocities (FIG. 26B left), as quantified in FIG. 26F. PTL had even less of an effect on NF myocytes, actually slightly prolonging the late phase of relaxation (FIG. 26B right, FIG. 26G). Yet in failing myocytes, both colch and PTL robustly improved shortening amplitude and velocity, and increased the speed of relaxation (FIG. 26C, FIG. 26F, and FIG. 26G). The average velocity traces from all cells are shown in FIG. 26D—neither colch nor PTL fully rescued contractile velocities of failing myocytes back to the NF benchmark, but restored ˜50% of lost function. Notably, the treatment-induced improvement in contractile kinetics from a given heart was inversely correlated with the initial contractile kinetics prior to treatment (FIG. 26E). Put differently, hearts with slowly contracting myocytes benefitted most from MT destabilization, raising the possibility of predicting therapeutic efficacy based on initial functional assessment. Of interest, myocytes from a failing heart with preserved ejection fraction (HFpEF) showed the slowest contractile duration and largest improvement in relaxation time upon suppression of detyrosinated MTs (FIG. 26C, right).

Upstream changes in excitation-contraction (EC) coupling could also contribute to the contractile alterations observed upon MT destabilization, for which there is precedent in the literature (58). We thus measured electrically stimulated [Ca²⁺]ᵢ transients in failing and NF myocytes with colch and PTL treatment (FIG. 27A-FIG. 27H). [Ca²⁺]ᵢ transients exhibited reduced amplitudes and slowed kinetics in failing myocytes, suggesting that defects in EC coupling likely contribute to their observed contractile dysfunction (FIG. 26A). However neither colch nor PTL treatment improved this impaired [Ca²⁺]ᵢ cycling (FIG. 26E), suggesting that the MT-dependent augmentation in contractility is primarily mechanical in origin. Colch had no significant effect on Ca²⁺ cycling in any group, while PTL treatment actually prolonged the [Ca²⁺]ᵢ transient decay phase in NF myocytes (FIG. 26C and FIG. 26G). As discussed further below, this slowed [Ca²⁺]ᵢ removal may explain the prolonged late relaxation in PTL treated NF myocytes (compare FIG. 26B and FIG. 27C). Regardless, from these studies we conclude that any improvement in contractility by colchicine or PTL treatment is not likely due to improved [Ca²⁺]ᵢ cycling.

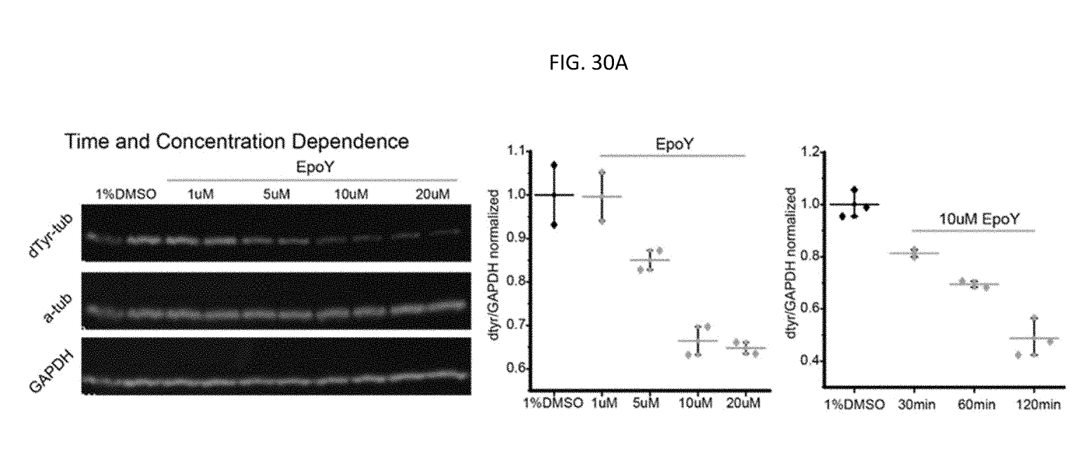

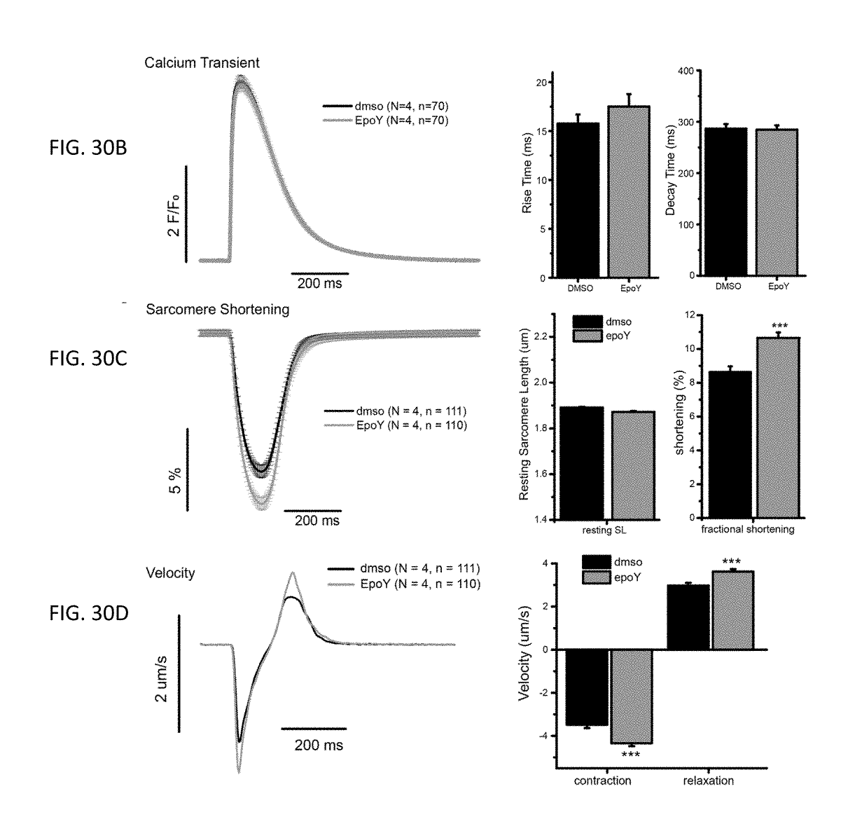

We also investigated the potential benefits of a recently identified inhibitor of TCP activity, epoY (17). These studies indicated that short-term treatment of rat myocytes with epoY reduced detyrosination and improved contractility (FIG. 30A-FIG. 30D).

### Genetic Modification of Tubulin Tyrosination Lowers Stiffness and Improves Contractility

We next aimed to validate these pharmacologic findings with a genetic approach, particularly given potential off-target effects of PTL. While the aforementioned TCP detyrosinates MTs, this process can be readily reversed by tubulin tyrosine ligase (TTL), which catalyzes the re-addition of the tyrosine residue to α-tubulin tails (for review see ref. 11). Adenoviral overexpression of TTL (AdV-TTL) for 48 hrs in cultured human cardiomyocytes decreased the density of dTyr-MTs and the proportion of total MTs that were detyrosinated, while also producing a slight drop in overall MT density compared to myocytes infected with a null encoding adenovirus (AdV-null) (FIG. 28A). TTL overexpression led to a significant increase in shortening amplitude and velocity in these myocytes, and increased relaxation velocities ˜2 fold (FIG. 28B-FIG. 28D). Stiffness measurements confirmed a large reduction in viscoelasticity in AdV-TTL myocytes compared to AdV-null (FIG. 28E and FIG. 28F), suggesting that contractile improvements are at least partly attributable to reduced internal resistance. While contractility experiments were typically performed at 0.5 Hz stimulation at room temperature, we also tested whether similar improvements were observed at physiological temperature and pacing frequency. At 37° C. all contractile parameters were similarly improved by TTL overexpression, and the magnitude of improvement was unchanged with either 0.5 or 1 Hz stimulation (FIG. 29A-FIG. 29C). Of note, the late phase of relaxation was significantly faster with TTL overexpression (FIGS. 28B and 28D right, FIG. 29A-FIG. 29C), in contrast to the prolongation of this component with PTL treatment. This suggests an off-target effect of PTL, as opposed to an on-target consequence of suppressing detyrosination.

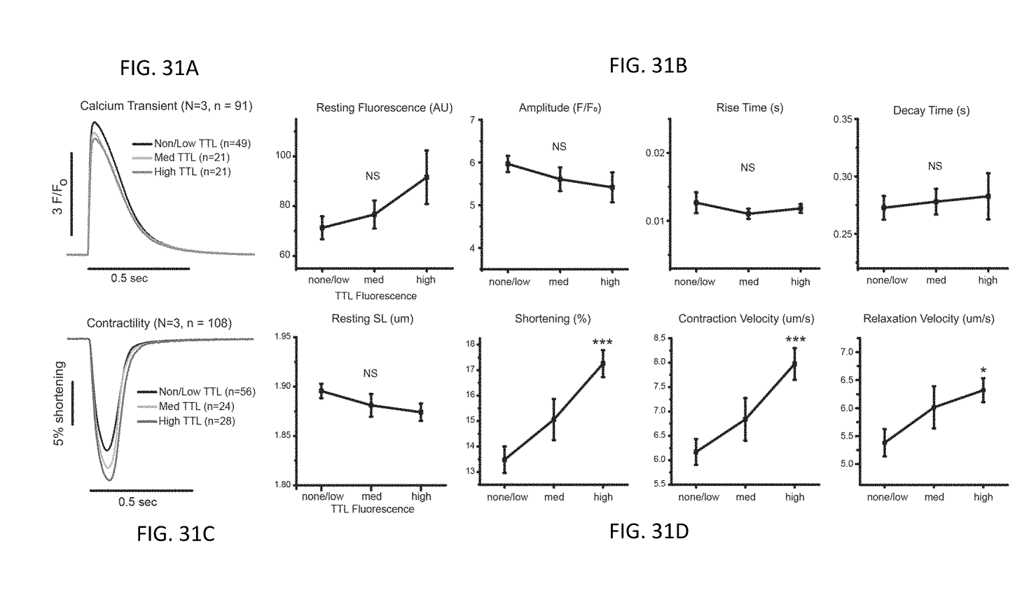

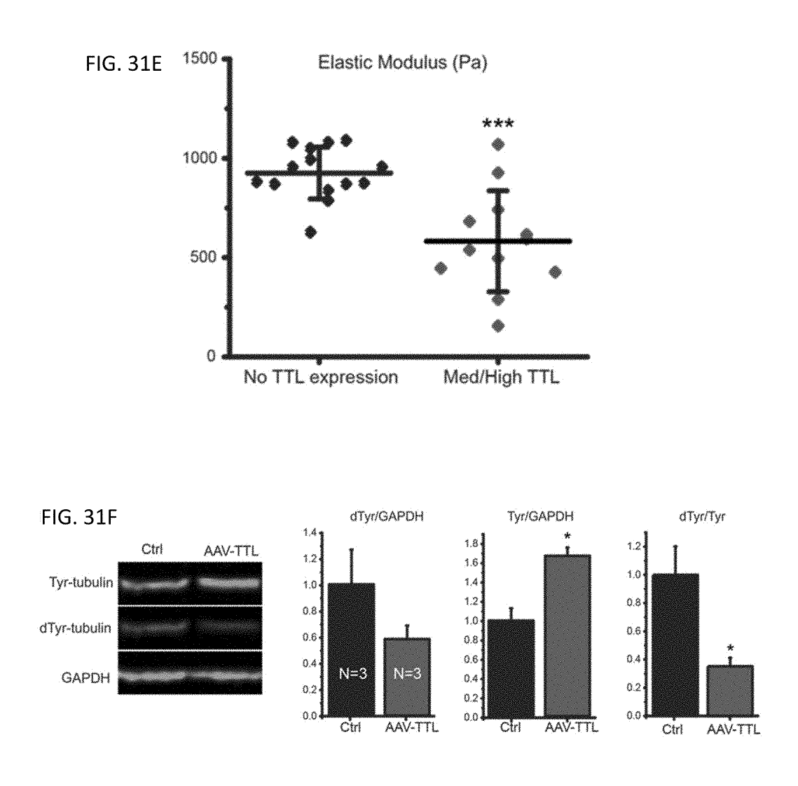

Additional studies using pericardial injection of rats with an AAV vector encoding TTL (AAV9-cTNT-TTL-mCherry) revealed that chronic overexpression of TTL in myocytes results in reduced stiffness and improved contractility (FIG. 31A-FIG. 31F). Retro-orbital injection of adult rats and mice with AAV9-cTNT-TTL showed similar delivery to the heart as pericardial injection of rat pups (data not shown). Thus, the use of AAV9 and a cTNT promoter results in almost exclusive cardiac myocyte specific expression, even following systemic delivery. We also performed studies using an adenoviral type 5 delivery vector with a CMV promoter to deliver TTL to isolated adult myocytes. These studies revealed improvements in contractile parameters similar to those observed using AAV9 medicated delivery (data not shown).

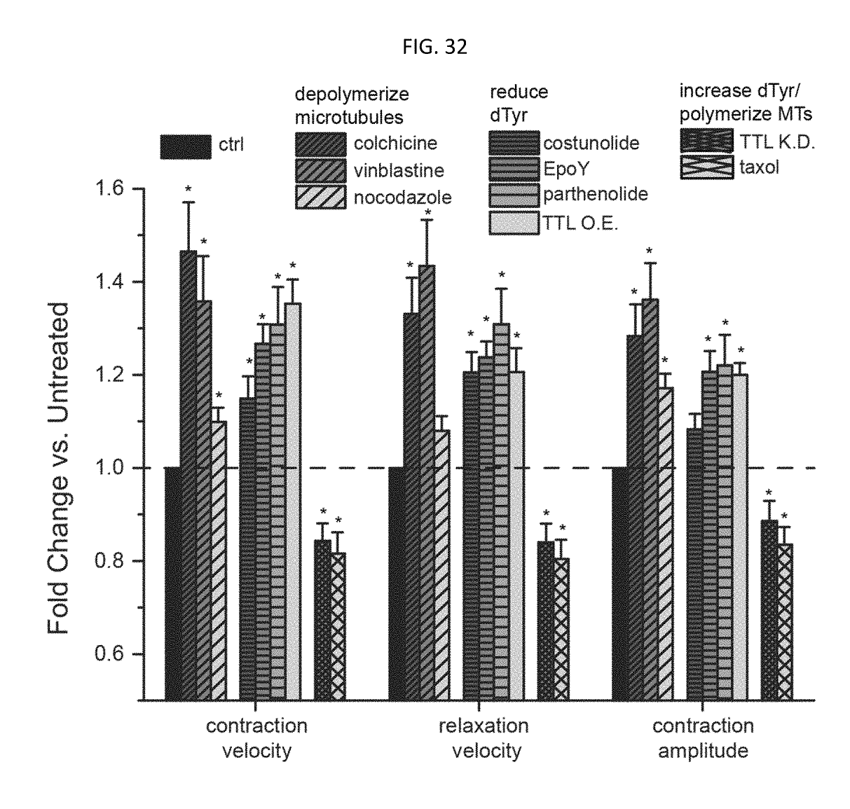

Finally, we also tested the effects of additional microtubule depolymerizing agents (nocodazole and vinblastine) and another TCP inhibitor, costunolide, and compared these results with our previous studies (FIG. 32). These agents also were effective to increase contraction amplitudes and velocities. Altogether, our results suggest that genetic and pharmacological manipulation of dTyr/Tyr balance represents a potent and specific tool to modulate contractility in human myocytes.

### Discussion

From the above we arrive at three major conclusions. First, the increased expression and stabilization of the non-sarcomeric cytoskeleton is a consistent feature of end stage heart failure. Second, an endogenously proliferated and modified MT network contributes viscoelastic resistance that impedes myocyte motion in heart failure. And third, destabilizing dTyr-MTs significantly lowers stiffness, enhances contractility and increases relaxation velocity in failing, human LV myocytes.

Our data suggest that whether dilated or hypertrophic in morphology, end-stage failing hearts share a surprisingly overlapping proteome. A dominant feature is the increased expression of cytoskeletal proteins, particularly IFs and MTs. These changes may initially be adaptive, perhaps to protect a heart under high mechanical stress, but become maladaptive when sufficiently progressed.

Our results in diverse cases of heart failure complement previous correlative studies in patients with aortic stenosis (59) and in animal models that show MT proliferation following a variety of disease stimuli, including (but not limited to) LV pressure overload (dog (35); mouse (60)), RV pressure overload (cats (8)), drug induced pulmonary hypertension (rats (61, 62); calf (63)), dystrophic cardiomyopathy (mouse (1, 64)) and diabetic cardiomyopathy (rats (65)). However, replication studies in LV overload models have also failed to show tubulin upregulation (guinea pig (66, 67); cats (68)), and contractility rescue via colchicine is far from consistent (61, 66, 68). Here, functional tests on human myocytes provide clinically relevant evidence of modified MTs as a therapeutic target in heart disease.

In aggregate, our data suggest that proliferated, detyrosinated MTs act as compression resistance elements to impair contraction in the failing heart. The efficacy of PTL treatment on stiffness and contractility—independent of an improvement in [Ca²⁺]ᵢ cycling or gross reduction in network density (12)—is best explained by a disruption in the interaction between dTyr-MTs and the sarcomere, which lowers the compression resistance provided by a cross-linked cytoskeletal network. In this light, the lesser effect of colch and PTL treatment on NF myocytes suggests that, in these cells, cytoskeletal resistance was insufficient to markedly impede contractility. This may be explained by a simple lack of MT density or detyrosination beyond a critical level, or resistance may be limited by the availability of MT interacting partners like desmin. In support of this hypothesis, myocytes lacking desmin appear insensitive to PTL, presumably due to the loss of a MT-IF crosslink. Thus, the efficacy of MT destabilization may depend on the proliferation of IFs that support the formation of a dense, cross-linked cytoskeletal network. Of note, both full-length desmin and lower MW products linked to aggregation were markedly upregulated in heart failure samples here and in a previous examination of dyssynchronous heart failure (57). The contribution of modified desmins to viscoelasticity and proteotoxicity demands further investigation.

We observed the largest improvements in contractile velocities with the tyrosination of MTs by AdV-TTL in cultured myocytes. Given the reduced MT network density and viscoelasticity in these cells, this improvement is likely attributable to reduced internal resistance. However, additional mechanisms may be at play, and warrant discussion when considering chronic effects of suppressing dTyr-MTs. Prolonged destabilization of MTs can prevent some of the adverse remodeling of the T-tubule system and subsequent disruption of E-C coupling that occurs in both heart failure and cell culture (60, 62). Further, suppressing dTyr has specifically been shown to reduce the generation of mechanical-stress induced reactive oxygen species, which could also benefit cell function by minimizing oxidative stress (1, 12). MTs also regulate mitochondrial positioning in cardiomyocytes (69), and broadly regulate vesicular transport, although any role of detyrosination in these processes remains to be explored.

Regardless of these confounding factors, the genetic modification of Tyr/dTyr balance represents a targeted approach with potential to sustainably improve both systolic and diastolic function. Our results suggest the improvement in myocyte function will likely correlate with the degree of myocyte stiffening and the slowing of contractile velocities, which will vary with disease etiology and severity. Further, while myocyte mechanics regulate whole organ function, this relationship is complex and will depend on numerous contributing factors. For example, in an ischemic, heavily fibrotic heart, tissue stiffness may be largely determined by the infiltrative extracellular matrix, potentially rendering a MT contribution nominal; yet in idiopathic or congenital myopathy with minimal fibrosis, myocyte specific changes, like those contributed by MTs and/or titin, would be predicted to play a more dominant role.

Finally, there is reason for optimism on the success of a MT-based inotropic strategy where others have failed. Currently available inotropes, such as dobutamine and milrinone, are endorsed for stabilization of patients with cardiogenic shock, as a bridge to transplant or long-term mechanical circulatory support, or as palliative therapy (72), because their long-term use may actually worsen patient outcomes (73). This is at least partly attributed to the increased metabolic cost and arrhythmia risk associated with chronically augmenting Ca²⁺ cycling or force production. Destabilizers of a dense cytoskeletal network would represent a new class of energetically neutral inotropes, which do not force the cell to burn more ATP, but simply lower the resistance the myocyte must work against to improve both systolic and diastolic performance.

### REFERENCES

1. B. L. Prosser, C. W. Ward, W. J. Lederer, X-ROS Signaling: Rapid Mechano-Chemo Transduction in Heart. Science. 333, 1440-1445 (2011).
2. B. L. Prosser, C. W. Ward, W. J. Lederer, X-ROS signaling is enhanced and graded by cyclic cardiomyocyte stretch. Cardiovascular Research (2013), doi:10.1093/cvr/cvt066.
3. M. R. Zile et al., Constitutive properties of adult mammalian cardiac muscle cells. Circulation. 98, 567-579 (1998).
4. S. Nishimura, Microtubules Modulate the Stiffness of Cardiomyocytes Against Shear Stress. Circulation Research. 98, 81-87 (2005).
5. H. L. Granzier, T. C. Irving, Passive tension in cardiac muscle: contribution of collagen, titin, microtubules, and intermediate filaments. Biophysical Journal. 68, 1027-1044 (1995).
6. C. P. Brangwynne, Microtubules can bear enhanced compressive loads in living cells because of lateral reinforcement. J. Cell Biol. 173, 733-741 (2006).
7. M. Mehrbod, M. R. K. Mofrad, On the significance of microtubule flexural behavior in cytoskeletal mechanics. PLoS ONE. 6, e25627 (2011).
8. H. Tsutsui, K. Ishihara, G. Cooper, Cytoskeletal role in the contractile dysfunction of hypertrophied myocardium. Science. 260, 682-687 (1993).
9. M. R. Zile et al., Role of microtubules in the contractile dysfunction of hypertrophied myocardium. JAC. 33, 250-260 (1999).
10. G. Cooper, Cytoskeletal networks and the regulation of cardiac contractility: microtubules, hypertrophy, and cardiac dysfunction. Am. J. Physiol. Heart Circ. Physiol. 291, H1003-14 (2006).
11. C. Janke, J. C. Bulinski, Post-translational regulation of the microtubule cytoskeleton: mechanisms and functions. Nat. Rev. Mol. Cell Biol. 12, 773-786 (2011).
12. J. P. Kerr et al., Detyrosinated microtubules modulate mechanotransduction in heart and skeletal muscle. Nat Commun. 1-14 (2015).
13. H. Sato et al., Microtubule stabilization in pressure overload cardiac hypertrophy. J. Cell Biol. 139, 963-973 (1997).
14. S. Belmadani, C. Poüs, R. Ventura-Clapier, R. Fischmeister, P.-F. Méry, Post-translational modifications of cardiac tubulin during chronic heart failure in the rat. Mol. Cell. Biochem. 237, 39-46 (2002).
15. E. White, Mechanical modulation of cardiac microtubules. Pflugers Arch. 462, 177-184 (2011).
16. G. Cooper, Cardiocyte cytoskeleton in hypertrophied myocardium. Heart Fail Rev. 5, 187-201 (2000).
17. C. Aillaud et al. Vasohibins/SVBP are tubulin carboxypeptidases (TCPs) that regulate neuron differentiation. Science 358, 1448-1453 (2017).
18. L. Cassimeris, P. Tran, Methods Cell Biol., in press, doi:10.1016/S0091-679X(10)97031-3.
19. G. Lukinavičius et al., Fluorogenic probes for live-cell imaging of the cytoskeleton. Nat Meth. 11, 731-733 (2014).
20. S. Belmadani, C. Poüs, R. Fischmeister, P.-F. Méry, Post-translational modifications of tubulin and microtubule stability in adult rat ventricular myocytes and immortalized HL-1 cardiomyocytes. Mol. Cell. Biochem. 258, 35-48 (2004).
21. C. Janke et al., Tubulin polyglutamylase enzymes are members of the TTL domain protein family. Science. 308, 1758-1762 (2005).
22. C. Erck et al., A vital role of tubulin-tyrosine-ligase for neuronal organization. Proc. Natl. Acad. Sci. U.S.A. 102, 7853-7858 (2005).
23. L. Peris et al., Motor-dependent microtubule disassembly driven by tubulin tyrosination. J. Cell Biol. 185, 1159-1166 (2009).
24. M. Sirajuddin, L. M. Rice, R. D. Vale, Regulation of microtubule motors by tubulin isotypes and post-translational modifications. Nat Cell Biol. 16, 335-344 (2014).
25. G. Kreitzer, G. Liao, G. G. Gundersen, Detyrosination of tubulin regulates the interaction of intermediate filaments with microtubules in vivo via a kinesin-dependent mechanism. Mol. Biol. Cell. 10, 1105-1118 (1999).
26. R. A. Whipple et al., Vimentin filaments support extension of tubulin-based microtentacles in detached breast tumor cells. Cancer Res. 68, 5678-5688 (2008).
27. X. Fonrose et al., Parthenolide inhibits tubulin carboxypeptidase activity. Cancer Res. 67, 3371-3378 (2007).
28. M. Helmes, K. Trombitás, H. Granzier. Titin develops restoring force in rat cardiac myocytes. Circulation Research. 79, 619-626 (1996).
29. R. E. Mahaffy, C. K. Shih, F. C. MacKintosh, J. Käs. Scanning probe-based frequency-dependent microrheology of polymer gels and biological cells. Phys. Rev. Lett. 85, 880-883 (2000).
30. P. Konieczny et al. Myofiber integrity depends on desmin network targeting to Z-disks and costameres via distinct plectin isoforms. J. Cell Biol. 181, 667-681 (2008).
31. G. Liao & G. G. Gundersen. Kinesin is a candidate for cross-bridging microtubules and intermediate filaments. Selective binding of kinesin to detyrosinated tubulin and vimentin. J. Biol. Chem. 273, 9797-9803 (1998).
32. G. Gurland & G. G. Gundersen. Stable, detyrosinated microtubules function to localize vimentin intermediate filaments in fibroblasts. J. Cell Biol. 131, 1275-1290 (1995).
33. R. J. Khairallah et al. Microtubules underlie dysfunction in duchenne muscular dystrophy. Sci Signal. 5, ra56 (2012).
34. M. Prager-Khoutorsky, A. Khoutorsky, C. W. Bourque. Unique interweaved microtubule scaffold mediates osmosensory transduction via physical interaction with TRPV 1. Neuron. 83, 866-878 (2014).
35. H. Tagawa et al. Cytoskeletal mechanics in pressure-overload cardiac hypertrophy. Circulation Research. 80, 281-289 (1997).
36. A. O'Neill et al. Sarcolemmal organization in skeletal muscle lacking desmin: evidence for cytokeratins associated with the membrane skeleton at costameres. Mol. Biol. Cell. 13, 2347-2359 (2002).
37. A. E. Tollefson, M. Kuppuswamy, E. V. Shashkova, K. Doronin, W. S. M. Wold. Preparation and titration of CsCl-banded adenovirus stocks. Methods Mol. Med. 130, 223-235 (2007).
38. Y. C. Fung. Biomechanics (Springer Science & Business Media, 2013).
39. H. Kojima, A. Ishijima, T. Yanagida. Direct measurement of stiffness of single actin filaments with and without tropomyosin by in vitro nanomanipulation. Proc. Natl. Acad. Sci. U.S.A. 91, 12962-12966 (1994).
40. A. Grützner et al. Modulation of titin-based stiffness by disulfide bonding in the cardiac titin N2-B unique sequence. Biophysical Journal. 97, 825-834 (2009).
41. O. Cazorla et al. Differential expression of cardiac titin isoforms and modulation of cellular stiffness. Circulation Research. 86, 59-67 (2000).
42. M. Das, A. J. Levine, F. C. MacKintosh. Buckling and force propagation along intracellular microtubules. EPL (Europhysics Letters) (2008).
43. M. A. Caporizzo et al. Strain-rate Dependence of Elastic Modulus Reveals Silver Nanoparticle Induced Cytotoxicity. Nanobiomedicine, 1 (2015).
44. R. M. Lang et al. Recommendations for chamber quantification. Eur. J. Echocardiogr. 7, 79-108 (2006).
45. K. Dipla, J. A. Mattiello, V. Jeevanandam, S.R. Houser, & K. B. Margulies. Myocyte recovery after mechanical circulatory support in humans with end-stage heart failure. Circulation 97, 2316-2322 (1998).
46. J. Simithy et al. Characterization of histone acylations links chromatin modifications with metabolism. Nature Communications 8, 1141 (2017).
47. J. Cox & M. Mann. MaxQuant enables high peptide identification rates, individualized p.p.b.-range mass accuracies and proteome-wide protein quantification. Nat. Biotechnol. 26, 1367-1372 (2008).
48. J. E. Elias, & S. P. Gygi. Target-decoy search strategy for increased confidence in large-scale protein identifications by mass spectrometry. Nat. Methods 4, 207-214 (2007).
49. M. A. Burke et al. Molecular profiling of dilated cardiomyopathy that progresses to heart failure. JCI Insight. 1, (2016).
50. J. Chen, E. E. Bardes, B. J. Aronow, & A. G. Jegga. ToppGene Suite for gene list enrichment analysis and candidate gene prioritization. Nucleic Acids Res. 37, W305-11 (2009).
51. E. G. Frolova et al. Thrombospondin-4 regulates fibrosis and remodeling of the myocardium in response to pressure overload. FASEB J. 26, 2363-2373 (2012).
52. J. D. Wulfkuhle et al. Domain analysis of supervillin, an F-actin bundling plasma membrane protein with functional nuclear localization signals. J. Cell Sci. 112 (Pt 13), 2125-2136 (1999).
53. M. Takahashi et al. Phenotypic consequences of beta1-tubulin expression and MAP4 decoration of microtubules in adult cardiocytes. Am. J. Physiol. Heart Circ. Physiol. 285, H2072-83 (2003).
54. G. Cheng et al. Basis for MAP4 dephosphorylation-related microtubule network densification in pressure overload cardiac hypertrophy. J. Biol. Chem. 285, 38125-38140 (2010).
55. A. E. Prota et al. Structural basis of tubulin tyrosination by tubulin tyrosine ligase. J. Cell Biol. 200, 259-270 (2013).
56. K. W. Prins et al. Dystrophin is a microtubule-associated protein. J. Cell Biol. 186, 363-369 (2009).
57. G. Agnetti et al. Desmin modifications associate with amyloid-like oligomers deposition in heart failure. Cardiovasc. Res. 102, 24-34 (2014).
58. A. M. Gomez, B. G. Kerfant, G. Vassort. Microtubule disruption modulates Ca²⁺ signaling in rat cardiac myocytes. Circ. Res. 86, 30-36 (2000).
59. M. R. Zile et al. Cardiocyte cytoskeleton in patients with left ventricular pressure overload hypertrophy. J. Am. Coll. Cardiol. 37, 1080-1084 (2001).
60. C. Zhang et al. Microtubule-Mediated Defects in Junctophilin-2 Trafficking Contribute to Myocyte T-Tubule Remodeling and Ca²⁺ Handling Dysfunction in Heart Failure. Circulation (2014).
61. R. Stones et al. Microtubule proliferation in right ventricular myocytes of rats with monocrotaline-induced pulmonary hypertension. J. Mol. Cell. Cardiol. 56, 91-96 (2013).
62. K. W. Prins et al. Colchicine Depolymerizes Microtubules, Increases Junctophilin-2, and Improves Right Ventricular Function in Experimental Pulmonary Arterial Hypertension. J. Am. Heart Assoc. 6, (2017).
63. M. S. Lemler et al. Myocyte cytoskeletal disorganization and right heart failure in hypoxia-induced neonatal pulmonary hypertension. Am. J. Physiol. Heart Circ. Physiol. 279, H1365-76 (2000).
64. K. W. Prins et al. Microtubule-Mediated Misregulation of Junctophilin-2 Underlies T-Tubule Disruptions and Calcium Mishandling in mdx Mice. JACC: Basic to Transl. Sci. 1, 122-130 (2016).
65. H. Shiels et al. Stable microtubules contribute to cardiac dysfunction in the streptozotocin-induced model of type 1 diabetes in the rat. Mol. Cell. Biochem. 294, 173-180 (2007).
66. J. F. Collins et al. The role of the cytoskeleton in left ventricular pressure overload hypertrophy and failure. J. Mol. Cell. Cardiol. 28, 1435-1443 (1996).
67. X. Wang et al. Chronic pressure overload cardiac hypertrophy and failure in guinea pigs: II. Cytoskeletal remodeling. J. Mol. Cell. Cardiol. 31, 319-331 (1999).
68. B. A. Bailey et al. Cellular basis of contractile derangements of hypertrophied feline ventricular myocytes. J. Mol. Cell. Cardiol. 29, 1823-1835 (1997).
69. M. Miragoli et al. Microtubule-Dependent Mitochondria Alignment Regulates Calcium Release in Response to Nanomechanical Stimulus in Heart Myocytes. Cell Rep 14, 140-151 (2016).
70. M. Rochdi et al. Pharmacokinetics and absolute bioavailability of colchicine after i.v. and oral administration in healthy human volunteers and elderly subjects. Eur. J. Clin. Pharmacol. 46, 351-354 (1994).
71. R. A. Terkeltaub. Clinical practice. Gout. N. Engl. J. Med. 349, 1647-1655 (2003).
72. C. W. Yancy et al. 2013 ACCF/AHA guideline for the management of heart failure: a report of the American College of Cardiology Foundation/American Heart Association Task Force on Practice Guidelines. J. Am. Coll. Cardiol. 62, e147-239 (2013).
73. M. S. Cuffe et al. Short-term intravenous milrinone for acute exacerbation of chronic heart failure: a randomized controlled trial. JAMA 287, 1541-1547 (2002).
74. H. Tagawa et al. Cytoskeletal role in the transition from compensated to decompensated hypertrophy during adult canine left ventricular pressure overloading. Cir. Res. 82, 751-761 (1998).
75. Y. Ishibashi et al. Role of microtubules in myocyte contractile dysfunction during cardiac hypertrophy in the rat. Am. J. Physiol. 271, H1978-87 (1996).

All patents, patent applications, and publications, priority documents, including U.S. Provisional Application No. 62/650,227, and references to GenBank or another publicly available sequences database cited throughout the disclosure, are expressly incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

1. A method for improving heart function in humans comprising treating a patient with a therapeutic which inhibits tubulin carboxypeptidase (TCP).

2. The method according to claim 1, wherein the therapeutic is a costunolide, a parthenolide, or epoY.

3. A method for treating heart failure in humans comprising dosing a patient with a therapeutic which interferes with detyrosinated microtubules in cardiomyocytes.

4. The method according to claim 3, wherein the therapeutic is a small molecule drug selected from one or more of: sesquiterpene lactones such as parthenolide (PTL) or costunolide, or PTL pro-drugs such as LC-1, or microtubule destabilizers including colchicine, vinblastine, and nocodazole.

5. The method according to claim 3, wherein the therapeutic comprises a nucleic acid encoding a tubulin tyrosine ligase (TTL) gene under the control of regulatory elements direct expression thereof.

6. The method according to claim 5, wherein the therapeutic is a non-viral gene delivery system.

7. The method according claim 5, wherein the non-viral delivery system comprises a liposomal reagent.

8. The method according to claim 5, wherein the therapeutic is a viral vector comprising the nucleic acid encoding the ttl gene.

9. The method according to claim 8, wherein the viral vector is a recombinant adenovirus, lentivirus, or adeno-associated virus.

10. The method according to claim 9, wherein the recombinant adeno-associated virus is selected from AAV1, AAV5, AAV6, AAV9.

11. A method for improving heart function in humans comprising delivering a composition comprising a therapeutic which increases cardiac microtubule tyrosination,

12. The method according to claim 11, wherein the therapeutic comprises a nucleic acid encoding a tubulin tyrosine ligase (TTL) gene under the control of regulatory elements direct expression thereof.

13. The method according claim 11, wherein the composition comprises a non-viral delivery system which comprises a liposomal reagent.

14. The method according to claim 12, wherein the therapeutic is a viral vector comprising the nucleic acid encoding the TTL gene.

15. The method according to claim 14, wherein the viral vector is a recombinant adenovirus, lentivirus, or adeno-associated virus.

16. The method according to claim 15, wherein the recombinant adeno-associated virus is selected from AAV1, AAV5, AAV6, AAV9.

17. A replication-defective vector comprising a tubulin tyrosine ligase (TTL) under the control of a regulatory control sequence which directs expression thereof in the heart.

18. The viral vector according to claim 17, wherein the vector is an adenovirus, a lentivirus, or an adeno-associated virus.


SEQUENCE LISTING (9 sequences)

SEQ ID NO: 1 - DNA, 34910 bp, Artificial Sequence
  synthetic construct
  virion (4)..(461): Human\Ad5,wt bases 1-458; includes 5′ R-ITR and packaging signal
  primer (364)..(387): pAD\forward\primer
  promoter (731)..(1318): CMV\promoter
  primer (1268)..(1288): CMV\forward\primer
  primer (1362)..(1381): T7\primer
  promoter (1362)..(1378): T7\promoter
  misc_feature (1411)..(1431): attB1
  CDS (1474)..(2637): TTL
  RBS (2671)..(3255): IRES
  CDS (3259)..(3936): DsRed2
  misc_feature (3951)..(3971): complement; attB2
  CDS (3998)..(4039): V5\epitope
  primer (4007)..(4027): complement; V5\reverse\primer
  CDS (4049)..(4057): 3\stops
  polyA_signal (4066)..(4337): TK\pA
  primer (4073)..(4091): complement; TK\pA\reverse\primer
  virion (4357)..(34905): Human\Ad5,wt bases 3513-35935,E3 region deleted; includes 3′L-ITR
  primer (4360)..(4383): complement; pAD\reverse\primer
taacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcggatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagcaggctccaccatgggaaccaattcagtcgactggatctggtaccatgtacccatacgacgtcccagactacgctatgtacaccttcgtggtacgcgatgagaacagcagcgtctacgccgaggtctcccggctgctcctcgccaccggccactggaagaggctgcggcgagacaaccccagattcaacctgatgctgggagagaggaatcggctgcccttcgggagactgggtcacgagcccgggctggtacagttggtgaattactacaggggtgctgacaaactgtgtcgcaaagcttctttagtgaagctaatcaagacaagccctgaactggctgagtcctgcacatggttccctgaatcttatgtgatttatccaaccaatctcaagactccagttgctccagcacagaatggaattcagccaccaatcagtaactcaaggacagatgaaagagaattctttctcgcctcttataacagaaagaaagaggatggagagggcaacgtttggattgcaaagtcatcagccggtgccaaaggtgaaggcattctcatctcctcagaggcttcagagcttctcgatttcatagacaaccagggccaagtgcacgtgatccagaaatatcttgagcaccctctgctgcttgagccaggtcatcgcaagtttgacatccgaagctgggtcttggtggatcatcagtataatatctacctctatagagagggtgtgcttcggactgcttcagaaccatatcatgttgataatttccaagacaaaacctgccatttgaccaatcactgcattcaaaaagagtattcaaagaactacgggaagtatgaagaaggaaatgaaatgttcttcaaggagttcaatcagtacctaacaagtgctttgaacattaccctagaaagtagtatcttactacaaatcaaacatataataaggaactgcctcctgagcgtggagcctgccattagcaccaagcacctcccttaccagagcttccagctcttcggctttgacttcatggtcgatgaggagctgaaggtgtggctcattgaggtcaacggtgcccctgcatgtgctcagaagctctatgcagaactgtgccaaggcatcgtggacatagccatttccagtgtcttcccacccccagatgtggagcaacctcagacccagccagctgccttcatcaagctgtgactcgagcctaagcttctagataagatgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtctatctacatggccaagaagcccgtgcagctgcccggctactactacgtggacgccaagctggacatcacctcccacaacgaggactacaccatcgtggagcagtacgagcgcaccgagggccgccaccacctgttcctgtagcggccatctagacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatccgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaat

SEQ ID NO: 2 - protein, 387 aa, Artificial Sequence
  Synthetic Construct
MYPYDVPDYAMYTFVVRDENSSVYAEVSRLLLATGHWKRLRRDNPRFNLMLGERNRLPFGRLGHEPGLVQLVNYYRGADKLCRKASLVKLIKTSPELAESCTWFPESYVIYPTNLKTPVAPAQNGIQPPISNSRTDEREFFLASYNRKKEDGEGNVWIAKSSAGAKGEGILISSEASELLDFIDNQGQVHVIQKYLEHPLLLEPGHRKFDIRSWVLVDHQYNIYLYREGVLRTASEPYHVDNFQDKTCHLTNHCIQKEYSKNYGKYEEGNEMFFKEFNQYLTSALNITLESSILLQIKHIIRNCLLSVEPAISTKHLPYQSFQLFGFDFMVDEELKVWLIEVNGAPACAQKLYAELCQGIVDIAISSVFPPPDVEQPQTQPAAFIKL

SEQ ID NO: 3 - protein, 225 aa, Artificial Sequence
  Synthetic Construct
MASSENVITEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVATVTQDSSLQDGCFIYKVKFIGVNFPSDGPVMQKKTMGWEASTERLYPRDGVLKGETHKALKLKDGGHYLVEFKSIYMAKKPVQLPGYYYVDAKLDITSHNEDYTIVEQYERTEGRHHLFL

SEQ ID NO: 4 - protein, 14 aa, Artificial Sequence
  Synthetic Construct
GKPIPNPLLGLDST

SEQ ID NO: 5 - protein, 377 aa, Homo sapiens
MYTFVVRDENSSVYAEVSRLLLATGHWKRLRRDNPRFNLMLGERNRLPFGRLGHEPGLVQLVNYYRGADKLCRKASLVKLIKTSPELAESCTWFPESYVIYPTNLKTPVAPAQNGIQPPISNSRTDEREFFLASYNRKKEDGEGNVWIAKSSAGAKGEGILISSEASELLDFIDNQGQVHVIQKYLEHPLLLEPGHRKFDIRSWVLVDHQYNIYLYREGVLRTASEPYHVDNFQDKTCHLTNHCIQKEYSKNYGKYEEGNEMFFKEFNQYLTSALNITLESSILLQIKHIIRNCLLSVEPAISTKHLPYQSFQLFGFDFMVDEELKVWLIEVNGAPACAQKLYAELCQGIVDIAISSVFPPPDVEQPQTQPAAFIKL

SEQ ID NO: 6 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - DNA, 21 bp, Homo sapiens
gcttcagaaccatatcatgtt

SEQ ID NO: 8 - DNA, 19 bp, Homo sapiens
gtgcacgtgatccagaaat

SEQ ID NO: 9 - protein, 14 aa, Artificial Sequence
  synthetic peptide
GKPIPNPLLGLDST